SEQUENCE LISTING (342 sequences)

SEQ ID NO: 1 - DNA, 366 bp, synthetic construct
  misc_feature 1..366: note = synthetic
  source 1..366: mol_type = other DNA
caggtgcatctggtggagtctgggggaggcgtggtccagcctgggaggtccctgagactctcctgtgcagcctctggattcaccttcagtaactatggcatacactgggtccgccaggttccaggcaaggggctggagtgggtggcagttatatcatatgatggaaataataaatactatggggactccgtgaagggccgattcaccatctctagagacaattccaagaacacactgtatctgcaaatgaacagcctgagacctgaggacacggctatctattactgtgcgaaagatcgtggcctggacacagctggtgactactttgactattggggccagggaaccctggtcaccgtctcctca

SEQ ID NO: 2 - protein, 122 aa, synthetic construct
  REGION 1..122: note = synthetic
QVHLVESGGGVVQPGRSLRLSCAASGFTFSNYGIHWVRQVPGKGLEWVAVISYDGNNKYYGDSVKGRFTISRDNSKNTLYLQMNSLRPEDTAIYYCAKDRGLDTAGDYFDYWGQGTLVTVSS

SEQ ID NO: 3 - DNA, 24 bp, synthetic construct
  misc_feature 1..24: note = synthetic
  source 1..24: mol_type = other DNA
ggattcaccttcagtaactatggc

SEQ ID NO: 4 - protein, 8 aa, synthetic construct
  REGION 1..8: note = synthetic
GFTFSNYG

SEQ ID NO: 5 - DNA, 24 bp, synthetic construct
  misc_feature 1..24: note = synthetic
  source 1..24: mol_type = other DNA
atatcatatgatggaaataataaa

SEQ ID NO: 6 - protein, 8 aa, synthetic construct
  REGION 1..8: note = synthetic
ISYDGNNK

SEQ ID NO: 7 - DNA, 45 bp, synthetic construct
  misc_feature 1..45: note = synthetic
  source 1..45: mol_type = other DNA
gcgaaagatcgtggcctggacacagctggtgactactttgactat

SEQ ID NO: 8 - protein, 15 aa, synthetic construct
  REGION 1..15: note = synthetic
AKDRGLDTAGDYFDY

SEQ ID NO: 9 - DNA, 318 bp, synthetic construct
  misc_feature 1..318: note = synthetic
  source 1..318: mol_type = other DNA
gaaatagtgatgacgcagtctccagccaccctgtctgtgtctacaggggaaagagccaccctctcctgcagggccagtcagaatattagtagcaaattggcctggtaccagcagaaacctggccaggctcccaggctcctcatttatggtgcatccaccagggcctctggtatcccagccagattctggggcagtgggtctgggacagagttcactctcaccatcagcagtctgcagtctgaagattttgcagttttttactgtctccagtataataactggtggacgttcggccaagggaccaaggtggaaatcaaa

SEQ ID NO: 10 - protein, 106 aa, synthetic construct
  REGION 1..106: note = synthetic
EIVMTQSPATLSVSTGERATLSCRASQNISSKLAWYQQKPGQAPRLLIYGASTRASGIPARFWGSGSGTEFTLTISSLQSEDFAVFYCLQYNNWWTFGQGTKVEIK

SEQ ID NO: 11 - DNA, 18 bp, synthetic construct
  misc_feature 1..18: note = synthetic
  source 1..18: mol_type = other DNA
cagaatattagtagcaaa

SEQ ID NO: 12 - protein, 6 aa, synthetic construct
  REGION 1..6: note = synthetic
QNISSK

SEQ ID NO: 13-14 - skipped (2 entries)

SEQ ID NO: 15 - DNA, 24 bp, synthetic construct
  misc_feature 1..24: note = synthetic
  source 1..24: mol_type = other DNA
ctccagtataataactggtggacg

SEQ ID NO: 16 - protein, 8 aa, synthetic construct
  REGION 1..8: note = synthetic
LQYNNWWT

SEQ ID NO: 17 - DNA, 1350 bp, synthetic construct
  misc_feature 1..1350: note = synthetic
  source 1..1350: mol_type = other DNA
caggtgcatctggtggagtctgggggaggcgtggtccagcctgggaggtccctgagactctcctgtgcagcctctggattcaccttcagtaactatggcatacactgggtccgccaggttccaggcaaggggctggagtgggtggcagttatatcatatgatggaaataataaatactatggggactccgtgaagggccgattcaccatctctagagacaattccaagaacacactgtatctgcaaatgaacagcctgagacctgaggacacggctatctattactgtgcgaaagatcgtggcctggacacagctggtgactactttgactattggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 18 - protein, 449 aa, synthetic construct
  REGION 1..449: note = synthetic
QVHLVESGGGVVQPGRSLRLSCAASGFTFSNYGIHWVRQVPGKGLEWVAVISYDGNNKYYGDSVKGRFTISRDNSKNTLYLQMNSLRPEDTAIYYCAKDRGLDTAGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 642 bp, synthetic construct
  misc_feature 1..642: note = synthetic
  source 1..642: mol_type = other DNA
gaaatagtgatgacgcagtctccagccaccctgtctgtgtctacaggggaaagagccaccctctcctgcagggccagtcagaatattagtagcaaattggcctggtaccagcagaaacctggccaggctcccaggctcctcatttatggtgcatccaccagggcctctggtatcccagccagattctggggcagtgggtctgggacagagttcactctcaccatcagcagtctgcagtctgaagattttgcagttttttactgtctccagtataataactggtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 213 aa, synthetic construct
  REGION 1..213: note = synthetic
EIVMTQSPATLSVSTGERATLSCRASQNISSKLAWYQQKPGQAPRLLIYGASTRASGIPARFWGSGSGTEFTLTISSLQSEDFAVFYCLQYNNWWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 345 bp, synthetic construct
  misc_feature 1..345: note = synthetic
  source 1..345: mol_type = other DNA
caggtgcagctacagcagtggggcgcaggactgttgaagccttcggagaccctgtccctcacctgcgctgtctataatgggtccttcagtagttactactggaactggatccgccagcccccggggaaggggctggagtggcttggagaaatcaatcatagaggaagcaccaactacaacccgtccctcacgagtcgagtcaccatatcagttgacacgtccaagaaccagttctccctgaagctgacctctgtgaccgccgcggacacggcaatatattattgtacggcgcatactcggggctttctatactggggccagggaaccctggtcaccgtctcctca

SEQ ID NO: 22 - protein, 115 aa, synthetic construct
  REGION 1..115: note = synthetic
QVQLQQWGAGLLKPSETLSLTCAVYNGSFSSYYWNWIRQPPGKGLEWLGEINHRGSTNYNPSLTSRVTISVDTSKNQFSLKLTSVTAADTAIYYCTAHTRGFLYWGQGTLVTVSS

SEQ ID NO: 23 - DNA, 24 bp, synthetic construct
  misc_feature 1..24: note = synthetic
  source 1..24: mol_type = other DNA
aatgggtccttcagtagttactac

SEQ ID NO: 24 - protein, 8 aa, synthetic construct
  REGION 1..8: note = synthetic
NGSFSSYY

SEQ ID NO: 25 - DNA, 21 bp, synthetic construct
  misc_feature 1..21: note = synthetic
  source 1..21: mol_type = other DNA
atcaatcatagaggaagcacc

SEQ ID NO: 26 - protein, 7 aa, synthetic construct
  REGION 1..7: note = synthetic
INHRGST

SEQ ID NO: 27 - DNA, 27 bp, synthetic construct
  misc_feature 1..27: note = synthetic
  source 1..27: mol_type = other DNA
acggcgcatactcggggctttctatac

SEQ ID NO: 28 - protein, 9 aa, synthetic construct
  REGION 1..9: note = synthetic
TAHTRGFLY

SEQ ID NO: 29 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccaggcgagtcaggacattaacatctatttaaattggtatcagcagcaaccagggaaagcccctaaactcctgatctacgatgtatccaatttggtaccgggggtcccatcaaggttcagtggtactggatctgggacacatttttccttcaccatcagcagcctgcaacctgaagatattgcaacatattactgtcaacactatgatgatctcccgctcactttcggcggagggaccacggtggcgatcaga

SEQ ID NO: 30 - protein, 107 aa, synthetic construct
  REGION 1..107: note = synthetic
DIQMTQSPSSLSASVGDRVTITCQASQDINIYLNWYQQQPGKAPKLLIYDVSNLVPGVPSRFSGTGSGTHFSFTISSLQPEDIATYYCQHYDDLPLTFGGGTTVAIR

SEQ ID NO: 31 - DNA, 18 bp, synthetic construct
  misc_feature 1..18: note = synthetic
  source 1..18: mol_type = other DNA
caggacattaacatctat

SEQ ID NO: 32 - protein, 6 aa, synthetic construct
  REGION 1..6: note = synthetic
QDINIY

SEQ ID NO: 33-34 - skipped (2 entries)

SEQ ID NO: 35 - DNA, 27 bp, synthetic construct
  misc_feature 1..27: note = synthetic
  source 1..27: mol_type = other DNA
caacactatgatgatctcccgctcact

SEQ ID NO: 36 - protein, 9 aa, synthetic construct
  REGION 1..9: note = synthetic
QHYDDLPLT

SEQ ID NO: 37 - DNA, 1329 bp, synthetic construct
  misc_feature 1..1329: note = synthetic
  source 1..1329: mol_type = other DNA
caggtgcagctacagcagtggggcgcaggactgttgaagccttcggagaccctgtccctcacctgcgctgtctataatgggtccttcagtagttactactggaactggatccgccagcccccggggaaggggctggagtggcttggagaaatcaatcatagaggaagcaccaactacaacccgtccctcacgagtcgagtcaccatatcagttgacacgtccaagaaccagttctccctgaagctgacctctgtgaccgccgcggacacggcaatatattattgtacggcgcatactcggggctttctatactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 38 - protein, 442 aa, synthetic construct
  REGION 1..442: note = synthetic
QVQLQQWGAGLLKPSETLSLTCAVYNGSFSSYYWNWIRQPPGKGLEWLGEINHRGSTNYNPSLTSRVTISVDTSKNQFSLKLTSVTAADTAIYYCTAHTRGFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - DNA, 645 bp, synthetic construct
  misc_feature 1..645: note = synthetic
  source 1..645: mol_type = other DNA
gacatccagatgacccagtctccatcctccctgtctgcatctgtaggagacagagtcaccatcacttgccaggcgagtcaggacattaacatctatttaaattggtatcagcagcaaccagggaaagcccctaaactcctgatctacgatgtatccaatttggtaccgggggtcccatcaaggttcagtggtactggatctgggacacatttttccttcaccatcagcagcctgcaacctgaagatattgcaacatattactgtcaacactatgatgatctcccgctcactttcggcggagggaccacggtggcgatcagacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 40 - protein, 214 aa, synthetic construct
  REGION 1..214: note = synthetic
DIQMTQSPSSLSASVGDRVTITCQASQDINIYLNWYQQQPGKAPKLLIYDVSNLVPGVPSRFSGTGSGTHFSFTISSLQPEDIATYYCQHYDDLPLTFGGGTTVAIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 345 bp, synthetic construct
  misc_feature 1..345: note = synthetic
  source 1..345: mol_type = other DNA
caggtgcagctacagcagtggggcgcaggactgttgaagccttcggagaccctgtccctcacctgcgctgtctataatgggtccttcagtagtttctactggagctggatccgccagcccccggggaaggggctggagtggcttggggaaatcaatcatagaggaagaaccaacaacaacccgtccctcgcgagtcgagtcaccatatcagttgacacgtccaagagccagttctccctgaggctgacctctgtgaccgccgcggacacggctacatattattgtacggcgcatactcggggctttctatactggggccagggaacccgggtcaccgtctcctca

SEQ ID NO: 42 - protein, 115 aa, synthetic construct
  REGION 1..115: note = synthetic
QVQLQQWGAGLLKPSETLSLTCAVYNGSFSSFYWSWIRQPPGKGLEWLGEINHRGRTNNNPSLASRVTISVDTSKSQFSLRLTSVTAADTATYYCTAHTRGFLYWGQGTRVTVSS

SEQ ID NO: 43 - DNA, 24 bp, synthetic construct
  misc_feature 1..24: note = synthetic
  source 1..24: mol_type = other DNA
aatgggtccttcagtagtttctac

SEQ ID NO: 44 - protein, 8 aa, synthetic construct
  REGION 1..8: note = synthetic
NGSFSSFY

SEQ ID NO: 45 - DNA, 21 bp, synthetic construct
  misc_feature 1..21: note = synthetic
  source 1..21: mol_type = other DNA
atcaatcatagaggaagaacc

SEQ ID NO: 46 - protein, 7 aa, synthetic construct
  REGION 1..7: note = synthetic
INHRGRT

SEQ ID NO: 47 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccaggcgagtcaggacattaacaactatttaaattggtatcagcagaaaccagggcaagcccctaacctcctgatctacgatgtatttaatttactaccgggggtcccatcaaggttcagtggaagtggatctgggacagatttttccttcaccatcagcagcctgcagcctgaagatattgcaacatattactgtcaacactatgatgatctcccgctcactttcggcggcgggaccacggtggcgatcaga

SEQ ID NO: 48 - protein, 107 aa, synthetic construct
  REGION 1..107: note = synthetic
DIQMTQSPSSLSASVGDRVTITCQASQDINNYLNWYQQKPGQAPNLLIYDVFNLLPGVPSRFSGSGSGTDFSFTISSLQPEDIATYYCQHYDDLPLTFGGGTTVAIR

SEQ ID NO: 49 - DNA, 18 bp, synthetic construct
  misc_feature 1..18: note = synthetic
  source 1..18: mol_type = other DNA
caggacattaacaactat

SEQ ID NO: 50 - protein, 6 aa, synthetic construct
  REGION 1..6: note = synthetic
QDINNY

SEQ ID NO: 51-52 - skipped (2 entries)

SEQ ID NO: 53 - DNA, 1329 bp, synthetic construct
  misc_feature 1..1329: note = synthetic
  source 1..1329: mol_type = other DNA
caggtgcagctacagcagtggggcgcaggactgttgaagccttcggagaccctgtccctcacctgcgctgtctataatgggtccttcagtagtttctactggagctggatccgccagcccccggggaaggggctggagtggcttggggaaatcaatcatagaggaagaaccaacaacaacccgtccctcgcgagtcgagtcaccatatcagttgacacgtccaagagccagttctccctgaggctgacctctgtgaccgccgcggacacggctacatattattgtacggcgcatactcggggctttctatactggggccagggaacccgggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 54 - protein, 442 aa, synthetic construct
  REGION 1..442: note = synthetic
QVQLQQWGAGLLKPSETLSLTCAVYNGSFSSFYWSWIRQPPGKGLEWLGEINHRGRTNNNPSLASRVTISVDTSKSQFSLRLTSVTAADTATYYCTAHTRGFLYWGQGTRVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - DNA, 645 bp, synthetic construct
  misc_feature 1..645: note = synthetic
  source 1..645: mol_type = other DNA
gacatccagatgacccagtctccatcctccctgtctgcatctgtaggagacagagtcaccatcacttgccaggcgagtcaggacattaacaactatttaaattggtatcagcagaaaccagggcaagcccctaacctcctgatctacgatgtatttaatttactaccgggggtcccatcaaggttcagtggaagtggatctgggacagatttttccttcaccatcagcagcctgcagcctgaagatattgcaacatattactgtcaacactatgatgatctcccgctcactttcggcggcgggaccacggtggcgatcagacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 56 - protein, 214 aa, synthetic construct
  REGION 1..214: note = synthetic
DIQMTQSPSSLSASVGDRVTITCQASQDINNYLNWYQQKPGQAPNLLIYDVFNLLPGVPSRFSGSGSGTDFSFTISSLQPEDIATYYCQHYDDLPLTFGGGTTVAIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - DNA, 345 bp, synthetic construct
  misc_feature 1..345: note = synthetic
  source 1..345: mol_type = other DNA
caggtgcagctacagcagtggggcgcaggactgttgaagccttcggagaccctgtccctcacctgcgctgtctataatgggtccttcagtagttactactggagctggatccgccagcccccggggaagggactggagtggcttggggaaatcaatcatagaggaagaaccaactacaacccgtccctcacgagtcgagtcaccatttcagttgacacgtccaagaaccagttctccctgaagctgccctctgtgaccgccgcagacacggccatatattattgttcggcacatactcggggctttctatattggggccagggaaccctggtcaccgtctcctca

SEQ ID NO: 58 - protein, 115 aa, synthetic construct
  REGION 1..115: note = synthetic
QVQLQQWGAGLLKPSETLSLTCAVYNGSFSSYYWSWIRQPPGKGLEWLGEINHRGRTNYNPSLTSRVTISVDTSKNQFSLKLPSVTAADTAIYYCSAHTRGFLYWGQGTLVTVSS

SEQ ID NO: 59 - DNA, 27 bp, synthetic construct
  misc_feature 1..27: note = synthetic
  source 1..27: mol_type = other DNA
tcggcacatactcggggctttctatat

SEQ ID NO: 60 - protein, 9 aa, synthetic construct
  REGION 1..9: note = synthetic
SAHTRGFLY

SEQ ID NO: 61 - DNA, 321 bp, synthetic construct
  misc_feature 1..321: note = synthetic
  source 1..321: mol_type = other DNA
gacatccagatgacccagtctccatcctccctgtctgcatctgttggagacagagtcaccatcacttgccaggcgagtcaggacattaacaactatttaaattggtatcagcagaaaccagggaaagcccctaagctcctgatctacgatgcatccaatttggtaccgggggtcccatcaaggttcagtggtactggatctgggacagatttttccttcaccatcagcagcctgcagcctgaagatattgcaacatattactgtcaacactattatgatctcccgctcactttcggcggagggaccacggtggcgatcagc

SEQ ID NO: 62 - protein, 107 aa, synthetic construct
  REGION 1..107: note = synthetic
DIQMTQSPSSLSASVGDRVTITCQASQDINNYLNWYQQKPGKAPKLLIYDASNLVPGVPSRFSGTGSGTDFSFTISSLQPEDIATYYCQHYYDLPLTFGGGTTVAIS

SEQ ID NO: 63-64 - skipped (2 entries)

SEQ ID NO: 65 - DNA, 27 bp, synthetic construct
  misc_feature 1..27: note = synthetic
  source 1..27: mol_type = other DNA
caacactattatgatctcccgctcact

SEQ ID NO: 66 - protein, 9 aa, synthetic construct
  REGION 1..9: note = synthetic
QHYYDLPLT

SEQ ID NO: 67 - DNA, 1329 bp, synthetic construct
  misc_feature 1..1329: note = synthetic
  source 1..1329: mol_type = other DNA
caggtgcagctacagcagtggggcgcaggactgttgaagccttcggagaccctgtccctcacctgcgctgtctataatgggtccttcagtagttactactggagctggatccgccagcccccggggaagggactggagtggcttggggaaatcaatcatagaggaagaaccaactacaacccgtccctcacgagtcgagtcaccatttcagttgacacgtccaagaaccagttctccctgaagctgccctctgtgaccgccgcagacacggccatatattattgttcggcacatactcggggctttctatattggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 68 - protein, 442 aa, synthetic construct
  REGION 1..442: note = synthetic
QVQLQQWGAGLLKPSETLSLTCAVYNGSFSSYYWSWIRQPPGKGLEWLGEINHRGRTNYNPSLTSRVTISVDTSKNQFSLKLPSVTAADTAIYYCSAHTRGFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69 - DNA, 645 bp, synthetic construct
  misc_feature 1..645: note = synthetic
  source 1..645: mol_type = other DNA
gacatccagatgacccagtctccatcctccctgtctgcatctgttggagacagagtcaccatcacttgccaggcgagtcaggacattaacaactatttaaattggtatcagcagaaaccagggaaagcccctaagctcctgatctacgatgcatccaatttggtaccgggggtcccatcaaggttcagtggtactggatctgggacagatttttccttcaccatcagcagcctgcagcctgaagatattgcaacatattactgtcaacactattatgatctcccgctcactttcggcggagggaccacggtggcgatcagccgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 70 - protein, 214 aa, synthetic construct
  REGION 1..214: note = synthetic
DIQMTQSPSSLSASVGDRVTITCQASQDINNYLNWYQQKPGKAPKLLIYDASNLVPGVPSRFSGTGSGTDFSFTISSLQPEDIATYYCQHYYDLPLTFGGGTTVAISRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccaggcgagtcaggacattaacaactatttaaattggtatcagcagaaaccagggaaagcccctaagctcctgatctacgatgcatccaatttggtaccgggggtcccatcaaggttcagtggtactggatctgggacagatttttccttcaccatcagcagcctgcagcctgaagatattgcaacatattactgtcaacactattatgatctcccgctcactttcggcggagggaccacggtggcgatcaga

SEQ ID NO: 72 - protein, 107 aa, synthetic construct
  REGION 1..107: note = synthetic
DIQMTQSPSSLSASVGDRVTITCQASQDINNYLNWYQQKPGKAPKLLIYDASNLVPGVPSRFSGTGSGTDFSFTISSLQPEDIATYYCQHYYDLPLTFGGGTTVAIR

SEQ ID NO: 73 - DNA, 645 bp, synthetic construct
  misc_feature 1..645: note = synthetic
  source 1..645: mol_type = other DNA
gacatccagatgacccagtctccatcctccctgtctgcatctgtaggagacagagtcaccatcacttgccaggcgagtcaggacattaacaactatttaaattggtatcagcagaaaccagggaaagcccctaagctcctgatctacgatgcatccaatttggtaccgggggtcccatcaaggttcagtggtactggatctgggacagatttttccttcaccatcagcagcctgcagcctgaagatattgcaacatattactgtcaacactattatgatctcccgctcactttcggcggagggaccacggtggcgatcagacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 74 - protein, 214 aa, synthetic construct
  REGION 1..214: note = synthetic
DIQMTQSPSSLSASVGDRVTITCQASQDINNYLNWYQQKPGKAPKLLIYDASNLVPGVPSRFSGTGSGTDFSFTISSLQPEDIATYYCQHYYDLPLTFGGGTTVAIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - DNA, 348 bp, synthetic construct
  misc_feature 1..348: note = synthetic
  source 1..348: mol_type = other DNA
gaaatacaactggtagagtctgggggtgacttggtccagcctggggggtccctgaaactctcctgtgcagcctctggattcaccttcagtgactctgctatgcactgggtccgccaggcttccgggaaagggctggagtgggttggccgtattagaaacaaagctaacacttacgcgacatcatacgctgcgtcggtgaaaggcaggttcaccgtctccacagatgattcaaagaccacggcgtatctgcagatgagatacctgaaaatcgaggacacggccatctattactgtactagtgaccaatttgactactggggccagggaaccctggtcaccgtctcctca

SEQ ID NO: 76 - protein, 116 aa, synthetic construct
  REGION 1..116: note = synthetic
EIQLVESGGDLVQPGGSLKLSCAASGFTFSDSAMHWVRQASGKGLEWVGRIRNKANTYATSYAASVKGRFTVSTDDSKTTAYLQMRYLKIEDTAIYYCTSDQFDYWGQGTLVTVSS

SEQ ID NO: 77 - DNA, 24 bp, synthetic construct
  misc_feature 1..24: note = synthetic
  source 1..24: mol_type = other DNA
ggattcaccttcagtgactctgct

SEQ ID NO: 78 - protein, 8 aa, synthetic construct
  REGION 1..8: note = synthetic
GFTFSDSA

SEQ ID NO: 79 - DNA, 30 bp, synthetic construct
  misc_feature 1..30: note = synthetic
  source 1..30: mol_type = other DNA
attagaaacaaagctaacacttacgcgaca

SEQ ID NO: 80 - protein, 10 aa, synthetic construct
  REGION 1..10: note = synthetic
IRNKANTYAT

SEQ ID NO: 81 - DNA, 21 bp, synthetic construct
  misc_feature 1..21: note = synthetic
  source 1..21: mol_type = other DNA
actagtgaccaatttgactac

SEQ ID NO: 82 - protein, 6 aa, synthetic construct
  REGION 1..6: note = synthetic
TSDQFD

SEQ ID NO: 83 - DNA, 336 bp, synthetic construct
  misc_feature 1..336: note = synthetic
  source 1..336: mol_type = other DNA
gatgttgtgatgactcagtctccactctccctgtccgccacccttggacagccggcctccatctcctgcaggtctagtcaaagcctcgtatacagtgatggaatcacctacttgacttggtttcaacagaggccaggccaatctccaacgcgcctgatttatcaggtttctaaccgggactctggagtcccagacagattcaacggcagtgggtcaggcactgattttacactgaacatcagcagggtggaggctgaagatgttggattttattactgcatggaaggaacacactggccgtggacgttcggccaagggaccaaggtggagctcaaa

SEQ ID NO: 84 - protein, 112 aa, synthetic construct
  REGION 1..112: note = synthetic
DVVMTQSPLSLSATLGQPASISCRSSQSLVYSDGITYLTWFQQRPGQSPTRLIYQVSNRDSGVPDRFNGSGSGTDFTLNISRVEAEDVGFYYCMEGTHWPWTFGQGTKVELK

SEQ ID NO: 85 - DNA, 33 bp, synthetic construct
  misc_feature 1..33: note = synthetic
  source 1..33: mol_type = other DNA
caaagcctcgtatacagtgatggaatcacctac

SEQ ID NO: 86 - protein, 11 aa, synthetic construct
  REGION 1..11: note = synthetic
QSLVYSDGITY

SEQ ID NO: 87-88 - skipped (2 entries)

SEQ ID NO: 89 - DNA, 27 bp, synthetic construct
  misc_feature 1..27: note = synthetic
  source 1..27: mol_type = other DNA
atggaaggaacacactggccgtggacg

SEQ ID NO: 90 - protein, 9 aa, synthetic construct
  REGION 1..9: note = synthetic
MEGTHWPWT

SEQ ID NO: 91 - DNA, 1332 bp, synthetic construct
  misc_feature 1..1332: note = synthetic
  source 1..1332: mol_type = other DNA
gaaatacaactggtagagtctgggggtgacttggtccagcctggggggtccctgaaactctcctgtgcagcctctggattcaccttcagtgactctgctatgcactgggtccgccaggcttccgggaaagggctggagtgggttggccgtattagaaacaaagctaacacttacgcgacatcatacgctgcgtcggtgaaaggcaggttcaccgtctccacagatgattcaaagaccacggcgtatctgcagatgagatacctgaaaatcgaggacacggccatctattactgtactagtgaccaatttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 92 - protein, 443 aa, synthetic construct
  REGION 1..443: note = synthetic
EIQLVESGGDLVQPGGSLKLSCAASGFTFSDSAMHWVRQASGKGLEWVGRIRNKANTYATSYAASVKGRFTVSTDDSKTTAYLQMRYLKIEDTAIYYCTSDQFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - DNA, 660 bp, synthetic construct
  misc_feature 1..660: note = synthetic
  source 1..660: mol_type = other DNA
gatgttgtgatgactcagtctccactctccctgtccgccacccttggacagccggcctccatctcctgcaggtctagtcaaagcctcgtatacagtgatggaatcacctacttgacttggtttcaacagaggccaggccaatctccaacgcgcctgatttatcaggtttctaaccgggactctggagtcccagacagattcaacggcagtgggtcaggcactgattttacactgaacatcagcagggtggaggctgaagatgttggattttattactgcatggaaggaacacactggccgtggacgttcggccaagggaccaaggtggagctcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 94 - protein, 219 aa, synthetic construct
  REGION 1..219: note = synthetic
DVVMTQSPLSLSATLGQPASISCRSSQSLVYSDGITYLTWFQQRPGQSPTRLIYQVSNRDSGVPDRFNGSGSGTDFTLNISRVEAEDVGFYYCMEGTHWPWTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - DNA, 342 bp, synthetic construct
  misc_feature 1..342: note = synthetic
  source 1..342: mol_type = other DNA
caggtgcagctggtggagtctgggggaggcttggtcaagcctggagggtccctgagactctcctgtgcagcctctggattcaccttcagtgactactatatgagttggatccgccaggctccagggaaggggctggagtggatttcttttattagtagaactggtagtaccaaatactacgcagactctgtgaagggccgattcaccatctccagggacaacgccaagaattcactgaatctacaaatgagcagcctgagagccgaggacacggccgtgtatttctgtgcgagagagccactacaacactggggccagggcaccctggttaccgtctcctca

SEQ ID NO: 96 - protein, 114 aa, synthetic construct
  REGION 1..114: note = synthetic
QVQLVESGGGLVKPGGSLRLSCAASGFTFSDYYMSWIRQAPGKGLEWISFISRTGSTKYYADSVKGRFTISRDNAKNSLNLQMSSLRAEDTAVYFCAREPLQHWGQGTLVTVSS

SEQ ID NO: 97 - DNA, 24 bp, synthetic construct
  misc_feature 1..24: note = synthetic
  source 1..24: mol_type = other DNA
ggattcaccttcagtgactactat

SEQ ID NO: 98 - protein, 8 aa, synthetic construct
  REGION 1..8: note = synthetic
GFTFSDYY

SEQ ID NO: 99 - DNA, 24 bp, synthetic construct
  misc_feature 1..24: note = synthetic
  source 1..24: mol_type = other DNA
attagtagaactggtagtaccaaa

SEQ ID NO: 100 - protein, 8 aa, synthetic construct
  REGION 1..8: note = synthetic
ISRTGSTK

SEQ ID NO: 101 - DNA, 21 bp, synthetic construct
  misc_feature 1..21: note = synthetic
  source 1..21: mol_type = other DNA
gcgagagagccactacaacac

SEQ ID NO: 102 - protein, 7 aa, synthetic construct
  REGION 1..7: note = synthetic
AREPLQH

SEQ ID NO: 103 - DNA, 339 bp, synthetic construct
  misc_feature 1..339: note = synthetic
  source 1..339: mol_type = other DNA
aatgttgtgctgacccagactccaatttcctcacctgtcatccttggacagccggcctccatctcctgcaggtctagtcaaagcctcgtacacagtgatggagacacctacttgagttggcttcatcagaggccaggccagcctccaagactcctaatttatacggtttctaaccgggtgtctggggtcccagacagattcagtggcagtggggcagggacagatttcacactgaaaatcagtagggtggaagctgaggatgtcggggtttattactgcatgcaaggaacacaatttcccgcgctcactttcggcggagggaccaaggtggagatcaaa

SEQ ID NO: 104 - protein, 113 aa, synthetic construct
  REGION 1..113: note = synthetic
NVVLTQTPISSPVILGQPASISCRSSQSLVHSDGDTYLSWLHQRPGQPPRLLIYTVSNRVSGVPDRFSGSGAGTDFTLKISRVEAEDVGVYYCMQGTQFPALTFGGGTKVEIK

SEQ ID NO: 105 - DNA, 33 bp, synthetic construct
  misc_feature 1..33: note = synthetic
  source 1..33: mol_type = other DNA
caaagcctcgtacacagtgatggagacacctac

SEQ ID NO: 106 - protein, 11 aa, synthetic construct
  REGION 1..11: note = synthetic
QSLVHSDGDTY

SEQ ID NO: 107-108 - skipped (2 entries)

SEQ ID NO: 109 - DNA, 30 bp, synthetic construct
  misc_feature 1..30: note = synthetic
  source 1..30: mol_type = other DNA
atgcaaggaacacaatttcccgcgctcact

SEQ ID NO: 110 - protein, 10 aa, synthetic construct
  REGION 1..10: note = synthetic
MQGTQFPALT

SEQ ID NO: 111 - DNA, 1326 bp, synthetic construct
  misc_feature 1..1326: note = synthetic
  source 1..1326: mol_type = other DNA
caggtgcagctggtggagtctgggggaggcttggtcaagcctggagggtccctgagactctcctgtgcagcctctggattcaccttcagtgactactatatgagttggatccgccaggctccagggaaggggctggagtggatttcttttattagtagaactggtagtaccaaatactacgcagactctgtgaagggccgattcaccatctccagggacaacgccaagaattcactgaatctacaaatgagcagcctgagagccgaggacacggccgtgtatttctgtgcgagagagccactacaacactggggccagggcaccctggttaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 112 - protein, 441 aa, synthetic construct
  REGION 1..441: note = synthetic
QVQLVESGGGLVKPGGSLRLSCAASGFTFSDYYMSWIRQAPGKGLEWISFISRTGSTKYYADSVKGRFTISRDNAKNSLNLQMSSLRAEDTAVYFCAREPLQHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 113 - DNA, 663 bp, synthetic construct
  misc_feature 1..663: note = synthetic
  source 1..663: mol_type = other DNA
aatgttgtgctgacccagactccaatttcctcacctgtcatccttggacagccggcctccatctcctgcaggtctagtcaaagcctcgtacacagtgatggagacacctacttgagttggcttcatcagaggccaggccagcctccaagactcctaatttatacggtttctaaccgggtgtctggggtcccagacagattcagtggcagtggggcagggacagatttcacactgaaaatcagtagggtggaagctgaggatgtcggggtttattactgcatgcaaggaacacaatttcccgcgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 114 - protein, 220 aa, synthetic construct
  REGION 1..220: note = synthetic
NVVLTQTPISSPVILGQPASISCRSSQSLVHSDGDTYLSWLHQRPGQPPRLLIYTVSNRVSGVPDRFSGSGAGTDFTLKISRVEAEDVGVYYCMQGTQFPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - DNA, 342 bp, synthetic construct
  misc_feature 1..342: note = synthetic
  source 1..342: mol_type = other DNA
caggtgcagctggttgagtctgggggaggcttggtcaagcctggagggtccctgagactctcctgtgcagcctctggattcaccttcagtgactactacatgagctggatccgccaggctccagggaagggactggagtgggtttcatatattagtagaactggtagtaccaaatattacgcagactctgtgaagggccgattcaccatctccagggacaacgccaaaaactcactgaatctacaaatgagcagcctgagagccgaggacacggccgtgtatttctgtgcgagagagccactacaacactggggccagggcaccctggttaccgtctcctca

SEQ ID NO: 116 - protein, 114 aa, synthetic construct
  REGION 1..114: note = synthetic
QVQLVESGGGLVKPGGSLRLSCAASGFTFSDYYMSWIRQAPGKGLEWVSYISRTGSTKYYADSVKGRFTISRDNAKNSLNLQMSSLRAEDTAVYFCAREPLQHWGQGTLVTVSS

SEQ ID NO: 117 - DNA, 24 bp, synthetic construct
  misc_feature 1..24: note = synthetic
  source 1..24: mol_type = other DNA
ggattcaccttcagtgactactac

SEQ ID NO: 118 - DNA, 339 bp, synthetic construct
  misc_feature 1..339: note = synthetic
  source 1..339: mol_type = other DNA
aaaattgtgctgacccagactccactctcctcacctgtcacccttggacagccggcctccatctcctgcaggtctagtcaaagccttgtacacagtgatggagacacctacttgagttggcttcaccagaggccaggccagcctccaagactcctaatatatacgatttctaaccggttgtctggggtcccagacagattcagtggcagtggggcagggacagatttcacactgaaaatcagtagggtggaagctgaggatgtcggggtttattactgcatgcaaggaacacaatttcccgcgctcactttcggcggagggaccaaggtggagatcaaa

SEQ ID NO: 119 - protein, 113 aa, synthetic construct
  REGION 1..113: note = synthetic
KIVLTQTPLSSPVTLGQPASISCRSSQSLVHSDGDTYLSWLHQRPGQPPRLLIYTISNRLSGVPDRFSGSGAGTDFTLKISRVEAEDVGVYYCMQGTQFPALTFGGGTKVEIK

SEQ ID NO: 120 - DNA, 33 bp, synthetic construct
  misc_feature 1..33: note = synthetic
  source 1..33: mol_type = other DNA
caaagccttgtacacagtgatggagacacctac

SEQ ID NO: 121-122 - skipped (2 entries)

SEQ ID NO: 123 - DNA, 1326 bp, synthetic construct
  misc_feature 1..1326: note = synthetic
  source 1..1326: mol_type = other DNA
caggtgcagctggttgagtctgggggaggcttggtcaagcctggagggtccctgagactctcctgtgcagcctctggattcaccttcagtgactactacatgagctggatccgccaggctccagggaagggactggagtgggtttcatatattagtagaactggtagtaccaaatattacgcagactctgtgaagggccgattcaccatctccagggacaacgccaaaaactcactgaatctacaaatgagcagcctgagagccgaggacacggccgtgtatttctgtgcgagagagccactacaacactggggccagggcaccctggttaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 124 - protein, 441 aa, synthetic construct
  REGION 1..441: note = synthetic
QVQLVESGGGLVKPGGSLRLSCAASGFTFSDYYMSWIRQAPGKGLEWVSYISRTGSTKYYADSVKGRFTISRDNAKNSLNLQMSSLRAEDTAVYFCAREPLQHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 125 - DNA, 663 bp, synthetic construct
  misc_feature 1..663: note = synthetic
  source 1..663: mol_type = other DNA
aaaattgtgctgacccagactccactctcctcacctgtcacccttggacagccggcctccatctcctgcaggtctagtcaaagccttgtacacagtgatggagacacctacttgagttggcttcaccagaggccaggccagcctccaagactcctaatatatacgatttctaaccggttgtctggggtcccagacagattcagtggcagtggggcagggacagatttcacactgaaaatcagtagggtggaagctgaggatgtcggggtttattactgcatgcaaggaacacaatttcccgcgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 126 - protein, 220 aa, synthetic construct
  REGION 1..220: note = synthetic
KIVLTQTPLSSPVTLGQPASISCRSSQSLVHSDGDTYLSWLHQRPGQPPRLLIYTISNRLSGVPDRFSGSGAGTDFTLKISRVEAEDVGVYYCMQGTQFPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - DNA, 345 bp, synthetic construct
  misc_feature 1..345: note = synthetic
  source 1..345: mol_type = other DNA
gagatgcagctggtggagtctgggggaggcttggtccagcctggggggtcgctgaaagtctcctgtgcagcctctgggttcaccctcagtgactcttctatacactgggtccgccaggcttccgggaaagggctggagtggattggccgtatcagaagcaaaccttacagttacgcgacagcatatgctgcgtcggtgaaaggcaggttcaccatttctagagatgattcaaagaacacggcgtttctgcaaatgagcggcctgaaaaccgaggacacggccgtctattattgtactgggggggatgacttctggggccagggaaccctggtcaccgtctcctca

SEQ ID NO: 128 - protein, 115 aa, synthetic construct
  REGION 1..115: note = synthetic
EMQLVESGGGLVQPGGSLKVSCAASGFTLSDSSIHWVRQASGKGLEWIGRIRSKPYSYATAYAASVKGRFTISRDDSKNTAFLQMSGLKTEDTAVYYCTGGDDFWGQGTLVTVSS

SEQ ID NO: 129 - DNA, 24 bp, synthetic construct
  misc_feature 1..24: note = synthetic
  source 1..24: mol_type = other DNA
gggttcaccctcagtgactcttct

SEQ ID NO: 130 - protein, 8 aa, synthetic construct
  REGION 1..8: note = synthetic
GFTLSDSS

SEQ ID NO: 131 - DNA, 30 bp, synthetic construct
  misc_feature 1..30: note = synthetic
  source 1..30: mol_type = other DNA
atcagaagcaaaccttacagttacgcgaca

SEQ ID NO: 132 - protein, 10 aa, synthetic construct
  REGION 1..10: note = synthetic
IRSKPYSYAT

SEQ ID NO: 133 - DNA, 18 bp, synthetic construct
  misc_feature 1..18: note = synthetic
  source 1..18: mol_type = other DNA
actgggggggatgacttc

SEQ ID NO: 134 - protein, 6 aa, synthetic construct
  REGION 1..6: note = synthetic
TGGDDF

SEQ ID NO: 135 - DNA, 318 bp, synthetic construct
  misc_feature 1..318: note = synthetic
  source 1..318: mol_type = other DNA
gacatccagatgacccagtctccttccaccctgtctgcatctgttggagacagaatcaccatcacttgccgggccagtcagagtattagtacttggttggcctggtatcagcagaaaccagggaaagcccctaagctcctgatctataaggcgtctagtttagaaggtggggtcccttcaaggttcagcggcagtggatctgggacagatttcactctcaccatcagcagcctgcagcctgatgattttgcaacttattactgccaacaatataatacttattggacgttcggccaagggaccaaggtggaaatcaaa

SEQ ID NO: 136 - protein, 106 aa, synthetic construct
  REGION 1..106: note = synthetic
DIQMTQSPSTLSASVGDRITITCRASQSISTWLAWYQQKPGKAPKLLIYKASSLEGGVPSRFSGSGSGTDFTLTISSLQPDDFATYYCQQYNTYWTFGQGTKVEIK

SEQ ID NO: 137 - DNA, 18 bp, synthetic construct
  misc_feature 1..18: note = synthetic
  source 1..18: mol_type = other DNA
cagagtattagtacttgg

SEQ ID NO: 138 - protein, 6 aa, synthetic construct
  REGION 1..6: note = synthetic
QSISTW

SEQ ID NO: 139-140 - skipped (2 entries)

SEQ ID NO: 141 - DNA, 24 bp, synthetic construct
  misc_feature 1..24: note = synthetic
  source 1..24: mol_type = other DNA
caacaatataatacttattggacg

SEQ ID NO: 142 - protein, 8 aa, synthetic construct
  REGION 1..8: note = synthetic
QQYNTYWT

SEQ ID NO: 143 - DNA, 1329 bp, synthetic construct
  misc_feature 1..1329: note = synthetic
  source 1..1329: mol_type = other DNA
gagatgcagctggtggagtctgggggaggcttggtccagcctggggggtcgctgaaagtctcctgtgcagcctctgggttcaccctcagtgactcttctatacactgggtccgccaggcttccgggaaagggctggagtggattggccgtatcagaagcaaaccttacagttacgcgacagcatatgctgcgtcggtgaaaggcaggttcaccatttctagagatgattcaaagaacacggcgtttctgcaaatgagcggcctgaaaaccgaggacacggccgtctattattgtactgggggggatgacttctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 144 - protein, 442 aa, synthetic construct
  REGION 1..442: note = synthetic
EMQLVESGGGLVQPGGSLKVSCAASGFTLSDSSIHWVRQASGKGLEWIGRIRSKPYSYATAYAASVKGRFTISRDDSKNTAFLQMSGLKTEDTAVYYCTGGDDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 145 - DNA, 642 bp, synthetic construct
  misc_feature 1..642: note = synthetic
  source 1..642: mol_type = other DNA
gacatccagatgacccagtctccttccaccctgtctgcatctgttggagacagaatcaccatcacttgccgggccagtcagagtattagtacttggttggcctggtatcagcagaaaccagggaaagcccctaagctcctgatctataaggcgtctagtttagaaggtggggtcccttcaaggttcagcggcagtggatctgggacagatttcactctcaccatcagcagcctgcagcctgatgattttgcaacttattactgccaacaatataatacttattggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 146 - protein, 213 aa, synthetic construct
  REGION 1..213: note = synthetic
DIQMTQSPSTLSASVGDRITITCRASQSISTWLAWYQQKPGKAPKLLIYKASSLEGGVPSRFSGSGSGTDFTLTISSLQPDDFATYYCQQYNTYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - DNA, 363 bp, synthetic construct
  misc_feature 1..363: note = synthetic
  source 1..363: mol_type = other DNA
gaggtgcagctggtggagtctgggggaggcttggtacagcctggggggtccctgagactctcctgtgcagcctctggattcacctttagcagctatgccatgagttgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgatcccgtataactggaactacggaggggcttttgatatctggggccaagggacaatggtcaccgtctcttca

SEQ ID NO: 148 - protein, 121 aa, synthetic construct
  REGION 1..121: note = synthetic
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAIPYNWNYGGAFDIWGQGTMVTVSS

SEQ ID NO: 149 - DNA, 24 bp, synthetic construct
  misc_feature 1..24: note = synthetic
  source 1..24: mol_type = other DNA
ggattcacctttagcagctatgcc

SEQ ID NO: 150 - protein, 8 aa, synthetic construct
  REGION 1..8: note = synthetic
GFTFSSYA

SEQ ID NO: 151 - DNA, 24 bp, synthetic construct
  misc_feature 1..24: note = synthetic
  source 1..24: mol_type = other DNA
attagtggtagtggtggtagcaca

SEQ ID NO: 152 - protein, 8 aa, synthetic construct
  REGION 1..8: note = synthetic
ISGSGGST

SEQ ID NO: 153 - DNA, 42 bp, synthetic construct
  misc_feature 1..42: note = synthetic
  source 1..42: mol_type = other DNA
gcgatcccgtataactggaactacggaggggcttttgatatc

SEQ ID NO: 154 - protein, 14 aa, synthetic construct
  REGION 1..14: note = synthetic
AIPYNWNYGGAFDI

SEQ ID NO: 155 - DNA, 324 bp, synthetic construct
  misc_feature 1..324: note = synthetic
  source 1..324: mol_type = other DNA
gaaattgtgatgacgcagtctccagccaccctgtctgtgtctccagggaaaagagccaccctctcctgcagggccagtcagagtgttagtagtaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcaacagtataataactggcctccgtacacttttggccaggggaccaagctggagatcaaa

SEQ ID NO: 156 - protein, 108 aa, synthetic construct
  REGION 1..108: note = synthetic
EIVMTQSPATLSVSPGKRATLSCRASQSVSSNLAWYQQKPGQAPRLLIYGASTRATGIPARFSGSGSGTEFTLTISSLQSEDFAVYYCQQYNNWPPYTFGQGTKLEIK

SEQ ID NO: 157 - DNA, 18 bp, synthetic construct
  misc_feature 1..18: note = synthetic
  source 1..18: mol_type = other DNA
cagagtgttagtagtaac

SEQ ID NO: 158 - protein, 6 aa, synthetic construct
  REGION 1..6: note = synthetic
QSVSSN

SEQ ID NO: 159 - DNA, 30 bp, synthetic construct
  misc_feature 1..30: note = synthetic
  source 1..30: mol_type = other DNA
caacagtataataactggcctccgtacact

SEQ ID NO: 160 - protein, 10 aa, synthetic construct
  REGION 1..10: note = synthetic
QQYNNWPPYT

SEQ ID NO: 161 - DNA, 1347 bp, synthetic construct
  misc_feature 1..1347: note = synthetic
  source 1..1347: mol_type = other DNA
gaggtgcagctggtggagtctgggggaggcttggtacagcctggggggtccctgagactctcctgtgcagcctctggattcacctttagcagctatgccatgagttgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgatcccgtataactggaactacggaggggcttttgatatctggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 162 - protein, 448 aa, synthetic construct
  REGION 1..448: note = synthetic
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAIPYNWNYGGAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 163 - DNA, 648 bp, synthetic construct
  misc_feature 1..648: note = synthetic
  source 1..648: mol_type = other DNA
gaaattgtgatgacgcagtctccagccaccctgtctgtgtctccagggaaaagagccaccctctcctgcagggccagtcagagtgttagtagtaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcaacagtataataactggcctccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 164 - protein, 215 aa, synthetic construct
  REGION 1..215: note = synthetic
EIVMTQSPATLSVSPGKRATLSCRASQSVSSNLAWYQQKPGQAPRLLIYGASTRATGIPARFSGSGSGTEFTLTISSLQSEDFAVYYCQQYNNWPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - DNA, 357 bp, synthetic construct
  misc_feature 1..357: note = synthetic
  source 1..357: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtgttggcacaaactttgcacagaagtttcagggcagggtcaccatgaccagggacacgtccatcagcacagcctacatggaactgagcaggctgagatctgacgacacggccgtgtattactgtacgagagatggggcagcagctggcctctttgactactggggccagggaaccctggtcaccgtctcctca

SEQ ID NO: 166 - protein, 119 aa, synthetic construct
  REGION 1..119: note = synthetic
QVQLVQSGAEVKKPGASVKVSCKASGYTFTGYYMHWVRQAPGQGLEWMGWINPNSVGTNFAQKFQGRVTMTRDTSISTAYMELSRLRSDDTAVYYCTRDGAAAGLFDYWGQGTLVTVSS

SEQ ID NO: 167 - DNA, 24 bp, synthetic construct
  misc_feature 1..24: note = synthetic
  source 1..24: mol_type = other DNA
ggatacaccttcaccggctactat

SEQ ID NO: 168 - protein, 8 aa, synthetic construct
  REGION 1..8: note = synthetic
GYTFTGYY

SEQ ID NO: 169 - DNA, 24 bp, synthetic construct
  misc_feature 1..24: note = synthetic
  source 1..24: mol_type = other DNA
atcaaccctaacagtgttggcaca

SEQ ID NO: 170 - protein, 8 aa, synthetic construct
  REGION 1..8: note = synthetic
INPNSVGT

SEQ ID NO: 171 - DNA, 36 bp, synthetic construct
  misc_feature 1..36: note = synthetic
  source 1..36: mol_type = other DNA
acgagagatggggcagcagctggcctctttgactac

SEQ ID NO: 172 - protein, 12 aa, synthetic construct
  REGION 1..12: note = synthetic
TRDGAAAGLFDY

SEQ ID NO: 173 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcgagtcagggcattaacaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtttaacagtgccccgctcactttcggcggagggaccaaggtggagatcaaa

SEQ ID NO: 174 - protein, 107 aa, synthetic construct
  REGION 1..107: note = synthetic
DIQMTQSPSSLSASVGDRVTITCRASQGINNYLAWYQQKPGKVPKLLIYAASTLQSGVPSRFSGSGSGTDFTLTISSLQPEDVATYYCQKFNSAPLTFGGGTKVEIK

SEQ ID NO: 175 - DNA, 18 bp, synthetic construct
  misc_feature 1..18: note = synthetic
  source 1..18: mol_type = other DNA
cagggcattaacaattat

SEQ ID NO: 176 - protein, 6 aa, synthetic construct
  REGION 1..6: note = synthetic
QGINNY

SEQ ID NO: 177-178 - skipped (2 entries)

SEQ ID NO: 179 - DNA, 27 bp, synthetic construct
  misc_feature 1..27: note = synthetic
  source 1..27: mol_type = other DNA
caaaagtttaacagtgccccgctcact

SEQ ID NO: 180 - protein, 9 aa, synthetic construct
  REGION 1..9: note = synthetic
QKFNSAPLT

SEQ ID NO: 181 - DNA, 1341 bp, synthetic construct
  misc_feature 1..1341: note = synthetic
  source 1..1341: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtgttggcacaaactttgcacagaagtttcagggcagggtcaccatgaccagggacacgtccatcagcacagcctacatggaactgagcaggctgagatctgacgacacggccgtgtattactgtacgagagatggggcagcagctggcctctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 182 - protein, 446 aa, synthetic construct
  REGION 1..446: note = synthetic
QVQLVQSGAEVKKPGASVKVSCKASGYTFTGYYMHWVRQAPGQGLEWMGWINPNSVGTNFAQKFQGRVTMTRDTSISTAYMELSRLRSDDTAVYYCTRDGAAAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 183 - DNA, 645 bp, synthetic construct
  misc_feature 1..645: note = synthetic
  source 1..645: mol_type = other DNA
gacatccagatgacccagtctccatcctccctgtctgcatctgtaggagacagagtcaccatcacttgccgggcgagtcagggcattaacaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtttaacagtgccccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 184 - protein, 214 aa, synthetic construct
  REGION 1..214: note = synthetic
DIQMTQSPSSLSASVGDRVTITCRASQGINNYLAWYQQKPGKVPKLLIYAASTLQSGVPSRFSGSGSGTDFTLTISSLQPEDVATYYCQKFNSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 185 - DNA, 348 bp, synthetic construct
  misc_feature 1..348: note = synthetic
  source 1..348: mol_type = other DNA
gaggtgcagctggtggagtctgggggaggcttggtacagcctggggggtccctgagactctcctgtgcagcctctggattcacctttagcacctatgccatgagctgggtccgccaggctccagggaaggggctggagtgggtctcagctattagtggtagtggtggtagtacatactacgcagactccgtgaagggccggttcaccatctccagagacaattccaagaacacgctgtatctgcaaatgaagagcctgagagtcgaggacacggccgtatattactgtgcgaattccccatcctggttcgacccctggggccagggaaccctggtcaccgtctcctca

SEQ ID NO: 186 - protein, 116 aa, synthetic construct
  REGION 1..116: note = synthetic
EVQLVESGGGLVQPGGSLRLSCAASGFTFSTYAMSWVRQAPGKGLEWVSAISGSGGSTYYADSVKGRFTISRDNSKNTLYLQMKSLRVEDTAVYYCANSPSWFDPWGQGTLVTVSS

SEQ ID NO: 187 - DNA, 24 bp, synthetic construct
  misc_feature 1..24: note = synthetic
  source 1..24: mol_type = other DNA
ggattcacctttagcacctatgcc

SEQ ID NO: 188 - protein, 8 aa, synthetic construct
  REGION 1..8: note = synthetic
GFTFSTYA

SEQ ID NO: 189 - DNA, 24 bp, synthetic construct
  misc_feature 1..24: note = synthetic
  source 1..24: mol_type = other DNA
attagtggtagtggtggtagtaca

SEQ ID NO: 190 - DNA, 27 bp, synthetic construct
  misc_feature 1..27: note = synthetic
  source 1..27: mol_type = other DNA
gcgaattccccatcctggttcgacccc

SEQ ID NO: 191 - protein, 9 aa, synthetic construct
  REGION 1..9: note = synthetic
ANSPSWFDP

SEQ ID NO: 192 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 193 - protein, 108 aa, synthetic construct
  REGION 1..108: note = synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 194 - DNA, 18 bp, synthetic construct
  misc_feature 1..18: note = synthetic
  source 1..18: mol_type = other DNA
cagagcattagcagctat

SEQ ID NO: 195 - protein, 6 aa, synthetic construct
  REGION 1..6: note = synthetic
QSISSY

SEQ ID NO: 196 - DNA, 30 bp, synthetic construct
  misc_feature 1..30: note = synthetic
  source 1..30: mol_type = other DNA
caacagagttacagtacccctccgatcacc

SEQ ID NO: 197 - protein, 10 aa, synthetic construct
  REGION 1..10: note = synthetic
QQSYSTPPIT

SEQ ID NO: 198 - DNA, 1332 bp, synthetic construct
  misc_feature 1..1332: note = synthetic
  source 1..1332: mol_type = other DNA
gaggtgcagctggtggagtctgggggaggcttggtacagcctggggggtccctgagactctcctgtgcagcctctggattcacctttagcacctatgccatgagctgggtccgccaggctccagggaaggggctggagtgggtctcagctattagtggtagtggtggtagtacatactacgcagactccgtgaagggccggttcaccatctccagagacaattccaagaacacgctgtatctgcaaatgaagagcctgagagtcgaggacacggccgtatattactgtgcgaattccccatcctggttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 199 - protein, 443 aa, synthetic construct
  REGION 1..443: note = synthetic
EVQLVESGGGLVQPGGSLRLSCAASGFTFSTYAMSWVRQAPGKGLEWVSAISGSGGSTYYADSVKGRFTISRDNSKNTLYLQMKSLRVEDTAVYYCANSPSWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 200 - DNA, 648 bp, synthetic construct
  misc_feature 1..648: note = synthetic
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 201 - protein, 215 aa, synthetic construct
  REGION 1..215: note = synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - DNA, 357 bp, synthetic construct
  misc_feature 1..357: note = synthetic
  source 1..357: mol_type = other DNA
gaagtgcagctggtggagtctgggggaggcttggtacagcctggcaggtccctgagactctcctgtgcagcctctggattcacctttgatgattatggcatgcactgggtccgacaagctccagggaagggcctggagtgggtctcagttattagttggaatagtgctaccatagactatgcggactctgtgaagggccgattcaccatctccagagacaacgccaagaattccctgtttctgcaaatgaacagtctgagacctgaggacacggccttgtattactgtgcaaagaaaaatagccttggctggttctttgactactggggccagggaaccctggtcactgtctcctca

SEQ ID NO: 203 - protein, 119 aa, synthetic construct
  REGION 1..119: note = synthetic
EVQLVESGGGLVQPGRSLRLSCAASGFTFDDYGMHWVRQAPGKGLEWVSVISWNSATIDYADSVKGRFTISRDNAKNSLFLQMNSLRPEDTALYYCAKKNSLGWFFDYWGQGTLVTVSS

SEQ ID NO: 204 - DNA, 24 bp, synthetic construct
  misc_feature 1..24: note = synthetic
  source 1..24: mol_type = other DNA
ggattcacctttgatgattatggc

SEQ ID NO: 205 - protein, 8 aa, synthetic construct
  REGION 1..8: note = synthetic
GFTFDDYG

SEQ ID NO: 206 - DNA, 24 bp, synthetic construct
  misc_feature 1..24: note = synthetic
  source 1..24: mol_type = other DNA
attagttggaatagtgctaccata

SEQ ID NO: 207 - protein, 8 aa, synthetic construct
  REGION 1..8: note = synthetic
ISWNSATI

SEQ ID NO: 208 - DNA, 36 bp, synthetic construct
  misc_feature 1..36: note = synthetic
  source 1..36: mol_type = other DNA
gcaaagaaaaatagccttggctggttctttgactac

SEQ ID NO: 209 - protein, 12 aa, synthetic construct
  REGION 1..12: note = synthetic
AKKNSLGWFFDY

SEQ ID NO: 210 - DNA, 339 bp, synthetic construct
  misc_feature 1..339: note = synthetic
  source 1..339: mol_type = other DNA
gacatcgtgatgacccagtctccagactccctggctgtgtctctgggcgagagggccaccatcaactgcaagtccagccagagtgttttatacagctccaacgataacaactacttagcttggtaccagcagaaaccaggacagcctcctaaactactcatttactgggcatctacccgggaatccggggtccctgaccgattcagtggcagcgggtctgggacagatttcactctcaccatcagcagcctgcaggctgaagatgtggcagtttattactgtcagcaatattatagtactcccctcactttcggcggagggaccaaggtggagatcaaa

SEQ ID NO: 211 - protein, 113 aa, synthetic construct
  REGION 1..113: note = synthetic
DIVMTQSPDSLAVSLGERATINCKSSQSVLYSSNDNNYLAWYQQKPGQPPKLLIYWASTRESGVPDRFSGSGSGTDFTLTISSLQAEDVAVYYCQQYYSTPLTFGGGTKVEIK

SEQ ID NO: 212 - DNA, 36 bp, synthetic construct
  misc_feature 1..36: note = synthetic
  source 1..36: mol_type = other DNA
cagagtgttttatacagctccaacgataacaactac

SEQ ID NO: 213 - protein, 12 aa, synthetic construct
  REGION 1..12: note = synthetic
QSVLYSSNDNNY

SEQ ID NO: 214-215 - skipped (2 entries)

SEQ ID NO: 216 - DNA, 27 bp, synthetic construct
  misc_feature 1..27: note = synthetic
  source 1..27: mol_type = other DNA
cagcaatattatagtactcccctcact

SEQ ID NO: 217 - protein, 9 aa, synthetic construct
  REGION 1..9: note = synthetic
QQYYSTPLT

SEQ ID NO: 218 - DNA, 1341 bp, synthetic construct
  misc_feature 1..1341: note = synthetic
  source 1..1341: mol_type = other DNA
gaagtgcagctggtggagtctgggggaggcttggtacagcctggcaggtccctgagactctcctgtgcagcctctggattcacctttgatgattatggcatgcactgggtccgacaagctccagggaagggcctggagtgggtctcagttattagttggaatagtgctaccatagactatgcggactctgtgaagggccgattcaccatctccagagacaacgccaagaattccctgtttctgcaaatgaacagtctgagacctgaggacacggccttgtattactgtgcaaagaaaaatagccttggctggttctttgactactggggccagggaaccctggtcactgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 219 - protein, 446 aa, synthetic construct
  REGION 1..446: note = synthetic
EVQLVESGGGLVQPGRSLRLSCAASGFTFDDYGMHWVRQAPGKGLEWVSVISWNSATIDYADSVKGRFTISRDNAKNSLFLQMNSLRPEDTALYYCAKKNSLGWFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 220 - DNA, 663 bp, synthetic construct
  misc_feature 1..663: note = synthetic
  source 1..663: mol_type = other DNA
gacatcgtgatgacccagtctccagactccctggctgtgtctctgggcgagagggccaccatcaactgcaagtccagccagagtgttttatacagctccaacgataacaactacttagcttggtaccagcagaaaccaggacagcctcctaaactactcatttactgggcatctacccgggaatccggggtccctgaccgattcagtggcagcgggtctgggacagatttcactctcaccatcagcagcctgcaggctgaagatgtggcagtttattactgtcagcaatattatagtactcccctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 221 - protein, 220 aa, synthetic construct
  REGION 1..220: note = synthetic
DIVMTQSPDSLAVSLGERATINCKSSQSVLYSSNDNNYLAWYQQKPGQPPKLLIYWASTRESGVPDRFSGSGSGTDFTLTISSLQAEDVAVYYCQQYYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - DNA, 351 bp, synthetic construct
  misc_feature 1..351: note = synthetic
  source 1..351: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaatctatgcacagaagttccagggcagagtcaccatgaccacagacacatccacgagcacagcctacatggagctgaggagcctgagatctgacgacacggccgtgtattactgtgcgagagatataaactggtacttcgatctctggggccgtggcaccctggtcactgtctcctca

SEQ ID NO: 223 - protein, 117 aa, synthetic construct
  REGION 1..117: note = synthetic
QVQLVQSGAEVKKPGASVKVSCKASGYTFTSYGISWVRQAPGQGLEWMGWISAYNGNTIYAQKFQGRVTMTTDTSTSTAYMELRSLRSDDTAVYYCARDINWYFDLWGRGTLVTVSS

SEQ ID NO: 224 - DNA, 24 bp, synthetic construct
  misc_feature 1..24: note = synthetic
  source 1..24: mol_type = other DNA
ggttacacctttaccagctatggt

SEQ ID NO: 225 - protein, 8 aa, synthetic construct
  REGION 1..8: note = synthetic
GYTFTSYG

SEQ ID NO: 226 - DNA, 24 bp, synthetic construct
  misc_feature 1..24: note = synthetic
  source 1..24: mol_type = other DNA
atcagcgcttacaatggtaacaca

SEQ ID NO: 227 - protein, 8 aa, synthetic construct
  REGION 1..8: note = synthetic
ISAYNGNT

SEQ ID NO: 228 - DNA, 30 bp, synthetic construct
  misc_feature 1..30: note = synthetic
  source 1..30: mol_type = other DNA
gcgagagatataaactggtacttcgatctc

SEQ ID NO: 229 - protein, 10 aa, synthetic construct
  REGION 1..10: note = synthetic
ARDINWYFDL

SEQ ID NO: 230 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagggcattagaaatgatttaggctggtttcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaa

SEQ ID NO: 231 - protein, 107 aa, synthetic construct
  REGION 1..107: note = synthetic
DIQMTQSPSSLSASVGDRVTITCRASQGIRNDLGWFQQKPGKAPKRLIYAASSLQSGVPSRFSGSGSGTEFTLTISSLQPEDFATYYCLQHNSYPWTFGQGTKVEIK

SEQ ID NO: 232 - DNA, 18 bp, synthetic construct
  misc_feature 1..18: note = synthetic
  source 1..18: mol_type = other DNA
cagggcattagaaatgat

SEQ ID NO: 233 - protein, 6 aa, synthetic construct
  REGION 1..6: note = synthetic
QGIRND

SEQ ID NO: 234 - DNA, 27 bp, synthetic construct
  misc_feature 1..27: note = synthetic
  source 1..27: mol_type = other DNA
ctacagcataatagttacccgtggacg

SEQ ID NO: 235 - protein, 9 aa, synthetic construct
  REGION 1..9: note = synthetic
LQHNSYPWT

SEQ ID NO: 236 - DNA, 1335 bp, synthetic construct
  misc_feature 1..1335: note = synthetic
  source 1..1335: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaatctatgcacagaagttccagggcagagtcaccatgaccacagacacatccacgagcacagcctacatggagctgaggagcctgagatctgacgacacggccgtgtattactgtgcgagagatataaactggtacttcgatctctggggccgtggcaccctggtcactgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 237 - protein, 444 aa, synthetic construct
  REGION 1..444: note = synthetic
QVQLVQSGAEVKKPGASVKVSCKASGYTFTSYGISWVRQAPGQGLEWMGWISAYNGNTIYAQKFQGRVTMTTDTSTSTAYMELRSLRSDDTAVYYCARDINWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 238 - DNA, 645 bp, synthetic construct
  misc_feature 1..645: note = synthetic
  source 1..645: mol_type = other DNA
gacatccagatgacccagtctccatcctccctgtctgcatctgtaggagacagagtcaccatcacttgccgggcaagtcagggcattagaaatgatttaggctggtttcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 239 - protein, 214 aa, synthetic construct
  REGION 1..214: note = synthetic
DIQMTQSPSSLSASVGDRVTITCRASQGIRNDLGWFQQKPGKAPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - DNA, 357 bp, synthetic construct
  misc_feature 1..357: note = synthetic
  source 1..357: mol_type = other DNA
gaagtgcagctggtggagtctgggggaggcttggtacagcctggcaggtccctgagactctcctgtgcagcctctggattcacctttgatgattatggcatgcactgggtccgacaagctccagggaagggcctggagtgggtctcagttattagttggaatggtgccaccatagactatgcggactctgtgaagggccgattcaccatctccagagacaacgccaagaattccctgtttctgcaaatgaacagtctgagacctgaggacacggccttgtattactgtgcaaagaaaaatagccttggctggttctttgactactggggccagggaaccctggtcaccgtctcctca

SEQ ID NO: 241 - protein, 119 aa, synthetic construct
  REGION 1..119: note = synthetic
EVQLVESGGGLVQPGRSLRLSCAASGFTFDDYGMHWVRQAPGKGLEWVSVISWNGATIDYADSVKGRFTISRDNAKNSLFLQMNSLRPEDTALYYCAKKNSLGWFFDYWGQGTLVTVSS

SEQ ID NO: 242 - DNA, 24 bp, synthetic construct
  misc_feature 1..24: note = synthetic
  source 1..24: mol_type = other DNA
attagttggaatggtgccaccata

SEQ ID NO: 243 - protein, 8 aa, synthetic construct
  REGION 1..8: note = synthetic
ISWNGATI

SEQ ID NO: 244 - DNA, 321 bp, synthetic construct
  misc_feature 1..321: note = synthetic
  source 1..321: mol_type = other DNA
gacatccagatgacccagtctccatcctccctgtctgcatttgtaggagacagagtcactatcacttgccgggcaagtcagggcattagaaatgatttaggctggtatcagcagaaaccagggaaaacccctaagcgcctgatctatactgcatccagtttgcgaagtggggtcccatcgaggttcagcggcagtggatctgggacagacttcactctcacaatcagcagcctgcagcctgaagattttgcaacttattactgtctgcagcataataattacccgtacacttttggccaggggaccaagctggagatcaaa

SEQ ID NO: 245 - protein, 107 aa, synthetic construct
  REGION 1..107: note = synthetic
DIQMTQSPSSLSAFVGDRVTITCRASQGIRNDLGWYQQKPGKTPKRLIYTASSLRSGVPSRFSGSGSGTDFTLTISSLQPEDFATYYCLQHNNYPYTFGQGTKLEIK

SEQ ID NO: 246-247 - skipped (2 entries)

SEQ ID NO: 248 - DNA, 27 bp, synthetic construct
  misc_feature 1..27: note = synthetic
  source 1..27: mol_type = other DNA
ctgcagcataataattacccgtacact

SEQ ID NO: 249 - protein, 9 aa, synthetic construct
  REGION 1..9: note = synthetic
LQHNNYPYT

SEQ ID NO: 250 - DNA, 1341 bp, synthetic construct
  misc_feature 1..1341: note = synthetic
  source 1..1341: mol_type = other DNA
gaagtgcagctggtggagtctgggggaggcttggtacagcctggcaggtccctgagactctcctgtgcagcctctggattcacctttgatgattatggcatgcactgggtccgacaagctccagggaagggcctggagtgggtctcagttattagttggaatggtgccaccatagactatgcggactctgtgaagggccgattcaccatctccagagacaacgccaagaattccctgtttctgcaaatgaacagtctgagacctgaggacacggccttgtattactgtgcaaagaaaaatagccttggctggttctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 251 - protein, 446 aa, synthetic construct
  REGION 1..446: note = synthetic
EVQLVESGGGLVQPGRSLRLSCAASGFTFDDYGMHWVRQAPGKGLEWVSVISWNGATIDYADSVKGRFTISRDNAKNSLFLQMNSLRPEDTALYYCAKKNSLGWFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 252 - DNA, 645 bp, synthetic construct
  misc_feature 1..645: note = synthetic
  source 1..645: mol_type = other DNA
gacatccagatgacccagtctccatcctccctgtctgcatttgtaggagacagagtcactatcacttgccgggcaagtcagggcattagaaatgatttaggctggtatcagcagaaaccagggaaaacccctaagcgcctgatctatactgcatccagtttgcgaagtggggtcccatcgaggttcagcggcagtggatctgggacagacttcactctcacaatcagcagcctgcagcctgaagattttgcaacttattactgtctgcagcataataattacccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 253 - protein, 214 aa, synthetic construct
  REGION 1..214: note = synthetic
DIQMTQSPSSLSAFVGDRVTITCRASQGIRNDLGWYQQKPGKTPKRLIYTASSLRSGVPSRFSGSGSGTDFTLTISSLQPEDFATYYCLQHNNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 254 - DNA, 366 bp, synthetic construct
  misc_feature 1..366: note = synthetic
  source 1..366: mol_type = other DNA
gaggtgcagctggtggagtctgggggaaacttggtacagccgggggggtccctgagactctcctgtgcagcctctggattcacctttagcaattatgccatgagttgggtccgccaggctccagggaaggggctggagtgggtctcaggtatcactggtaatggtgttaacacatattattcagtctccgtgaagggccggttcaccatctccagagacaattccaagaacacgctgtttctggaaatgaacagcctgagagccgaggacacggccatatattactgtgtgaaagaaaggggccacagctggttcggggactggttcgacccctggggccagggaaccctggtcaccgtctcctca

SEQ ID NO: 255 - protein, 122 aa, synthetic construct
  REGION 1..122: note = synthetic
EVQLVESGGNLVQPGGSLRLSCAASGFTFSNYAMSWVRQAPGKGLEWVSGITGNGVNTYYSVSVKGRFTISRDNSKNTLFLEMNSLRAEDTAIYYCVKERGHSWFGDWFDPWGQGTLVTVSS

SEQ ID NO: 256 - DNA, 24 bp, synthetic construct
  misc_feature 1..24: note = synthetic
  source 1..24: mol_type = other DNA
ggattcacctttagcaattatgcc

SEQ ID NO: 257 - protein, 8 aa, synthetic construct
  REGION 1..8: note = synthetic
GFTFSNYA

SEQ ID NO: 258 - DNA, 24 bp, synthetic construct
  misc_feature 1..24: note = synthetic
  source 1..24: mol_type = other DNA
atcactggtaatggtgttaacaca

SEQ ID NO: 259 - protein, 8 aa, synthetic construct
  REGION 1..8: note = synthetic
ITGNGVNT

SEQ ID NO: 260 - DNA, 45 bp, synthetic construct
  misc_feature 1..45: note = synthetic
  source 1..45: mol_type = other DNA
gtgaaagaaaggggccacagctggttcggggactggttcgacccc

SEQ ID NO: 261 - protein, 15 aa, synthetic construct
  REGION 1..15: note = synthetic
VKERGHSWFGDWFDP

SEQ ID NO: 262 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgtcggtcaagtcagagtgttatcagctatctaaattggtatcagcagaaaccaggaaaagcccctaaactcctgatatttggtgcatccagtttaataagtggggtcccatcaaggttcagtggcagtggatctgggacagatttcactctcaccatcgtcagtctgcagcctgaagatttcgcagtttacttctgtcaacagaattaccttcccccgctcactttcggcggagggaccaaggtggagatcaaa

SEQ ID NO: 263 - protein, 107 aa, synthetic construct
  REGION 1..107: note = synthetic
DIQMTQSPSSLSASVGDRVTITCRSSQSVISYLNWYQQKPGKAPKLLIFGASSLISGVPSRFSGSGSGTDFTLTIVSLQPEDFAVYFCQQNYLPPLTFGGGTKVEIK

SEQ ID NO: 264 - DNA, 18 bp, synthetic construct
  misc_feature 1..18: note = synthetic
  source 1..18: mol_type = other DNA
cagagtgttatcagctat

SEQ ID NO: 265 - protein, 6 aa, synthetic construct
  REGION 1..6: note = synthetic
QSVISY

SEQ ID NO: 266 - DNA, 27 bp, synthetic construct
  misc_feature 1..27: note = synthetic
  source 1..27: mol_type = other DNA
caacagaattaccttcccccgctcact

SEQ ID NO: 267 - protein, 9 aa, synthetic construct
  REGION 1..9: note = synthetic
QQNYLPPLT

SEQ ID NO: 268 - DNA, 1350 bp, synthetic construct
  misc_feature 1..1350: note = synthetic
  source 1..1350: mol_type = other DNA
gaggtgcagctggtggagtctgggggaaacttggtacagccgggggggtccctgagactctcctgtgcagcctctggattcacctttagcaattatgccatgagttgggtccgccaggctccagggaaggggctggagtgggtctcaggtatcactggtaatggtgttaacacatattattcagtctccgtgaagggccggttcaccatctccagagacaattccaagaacacgctgtttctggaaatgaacagcctgagagccgaggacacggccatatattactgtgtgaaagaaaggggccacagctggttcggggactggttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 269 - protein, 449 aa, synthetic construct
  REGION 1..449: note = synthetic
EVQLVESGGNLVQPGGSLRLSCAASGFTFSNYAMSWVRQAPGKGLEWVSGITGNGVNTYYSVSVKGRFTISRDNSKNTLFLEMNSLRAEDTAIYYCVKERGHSWFGDWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 270 - DNA, 645 bp, synthetic construct
  misc_feature 1..645: note = synthetic
  source 1..645: mol_type = other DNA
gacatccagatgacccagtctccatcctccctgtctgcatctgtaggagacagagtcaccatcacttgtcggtcaagtcagagtgttatcagctatctaaattggtatcagcagaaaccaggaaaagcccctaaactcctgatatttggtgcatccagtttaataagtggggtcccatcaaggttcagtggcagtggatctgggacagatttcactctcaccatcgtcagtctgcagcctgaagatttcgcagtttacttctgtcaacagaattaccttcccccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 271 - protein, 214 aa, synthetic construct
  REGION 1..214: note = synthetic
DIQMTQSPSSLSASVGDRVTITCRSSQSVISYLNWYQQKPGKAPKLLIFGASSLISGVPSRFSGSGSGTDFTLTIVSLQPEDFAVYFCQQNYLPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 272 - DNA, 366 bp, synthetic construct
  misc_feature 1..366: note = synthetic
  source 1..366: mol_type = other DNA
gaggtgcagctggtggagtctgggggaaacttggtacagccgggggggtccctgagactctcctgtgcagcctctggattcacctttagcaattatgccatgagttgggtccgccaggctccagggaaggggctggagtgggtctcaggtatcactggtaatggtattaaaacctactactcagtctccgcgaagggccggttcaccatctccagagacaattccaagaacacgctgtttctgcaaatgaacagcctgagagccgaggacacggccatatattactgtgtgaaagaaaggggccacagctggttcggggactggttcgacccctggggccagggaaccctggtcaccgtctcctca

SEQ ID NO: 273 - protein, 122 aa, synthetic construct
  REGION 1..122: note = synthetic
EVQLVESGGNLVQPGGSLRLSCAASGFTFSNYAMSWVRQAPGKGLEWVSGITGNGIKTYYSVSAKGRFTISRDNSKNTLFLQMNSLRAEDTAIYYCVKERGHSWFGDWFDPWGQGTLVTVSS

SEQ ID NO: 274 - DNA, 24 bp, synthetic construct
  misc_feature 1..24: note = synthetic
  source 1..24: mol_type = other DNA
atcactggtaatggtattaaaacc

SEQ ID NO: 275 - protein, 8 aa, synthetic construct
  REGION 1..8: note = synthetic
ITGNGIKT

SEQ ID NO: 276 - DNA, 336 bp, synthetic construct
  misc_feature 1..336: note = synthetic
  source 1..336: mol_type = other DNA
gatattgtgatgacccagactccactctcttcacctgtcacccttggacagccggcctccatctcctgcaggtctagtcaaggcctcgtaaacagtgatggaaacacctatttgagttggcttcaccagaggccaggccagcctccaagactcctaatttatgagatttctaaccgtttctctggagtcccagacagattcagtggcagtggggctgggacagatttcacactgaaaatcagtagggtggaagctgaggatgtcggattttattactgtatgcaatctacacaatttcctctcactttcggcggagggaccaaggtggagatcaaa

SEQ ID NO: 277 - protein, 112 aa, synthetic construct
  REGION 1..112: note = synthetic
DIVMTQTPLSSPVTLGQPASISCRSSQGLVNSDGNTYLSWLHQRPGQPPRLLIYEISNRFSGVPDRFSGSGAGTDFTLKISRVEAEDVGFYYCMQSTQFPLTFGGGTKVEIK

SEQ ID NO: 278 - DNA, 33 bp, synthetic construct
  misc_feature 1..33: note = synthetic
  source 1..33: mol_type = other DNA
caaggcctcgtaaacagtgatggaaacacctat

SEQ ID NO: 279 - protein, 11 aa, synthetic construct
  REGION 1..11: note = synthetic
QGLVNSDGNTY

SEQ ID NO: 280-281 - skipped (2 entries)

SEQ ID NO: 282 - DNA, 27 bp, synthetic construct
  misc_feature 1..27: note = synthetic
  source 1..27: mol_type = other DNA
atgcaatctacacaatttcctctcact

SEQ ID NO: 283 - protein, 9 aa, synthetic construct
  REGION 1..9: note = synthetic
MQSTQFPLT

SEQ ID NO: 284 - DNA, 1350 bp, synthetic construct
  misc_feature 1..1350: note = synthetic
  source 1..1350: mol_type = other DNA
gaggtgcagctggtggagtctgggggaaacttggtacagccgggggggtccctgagactctcctgtgcagcctctggattcacctttagcaattatgccatgagttgggtccgccaggctccagggaaggggctggagtgggtctcaggtatcactggtaatggtattaaaacctactactcagtctccgcgaagggccggttcaccatctccagagacaattccaagaacacgctgtttctgcaaatgaacagcctgagagccgaggacacggccatatattactgtgtgaaagaaaggggccacagctggttcggggactggttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 285 - protein, 449 aa, synthetic construct
  REGION 1..449: note = synthetic
EVQLVESGGNLVQPGGSLRLSCAASGFTFSNYAMSWVRQAPGKGLEWVSGITGNGIKTYYSVSAKGRFTISRDNSKNTLFLQMNSLRAEDTAIYYCVKERGHSWFGDWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 286 - DNA, 660 bp, synthetic construct
  misc_feature 1..660: note = synthetic
  source 1..660: mol_type = other DNA
gatattgtgatgacccagactccactctcttcacctgtcacccttggacagccggcctccatctcctgcaggtctagtcaaggcctcgtaaacagtgatggaaacacctatttgagttggcttcaccagaggccaggccagcctccaagactcctaatttatgagatttctaaccgtttctctggagtcccagacagattcagtggcagtggggctgggacagatttcacactgaaaatcagtagggtggaagctgaggatgtcggattttattactgtatgcaatctacacaatttcctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 287 - protein, 219 aa, synthetic construct
  REGION 1..219: note = synthetic
DIVMTQTPLSSPVTLGQPASISCRSSQGLVNSDGNTYLSWLHQRPGQPPRLLIYEISNRFSGVPDRFSGSGAGTDFTLKISRVEAEDVGFYYCMQSTQFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 288 - DNA, 366 bp, synthetic construct
  misc_feature 1..366: note = synthetic
  source 1..366: mol_type = other DNA
gaggtgcagctggtggagtctgggggaaacttggtacagccgggggggtccctgagactctcctgtgcagcctctggattcacctttagcaattatgccatgagttgggtccgccaggctccagggaagggactggagtgggtctcaggtatcactggcaatggtattaatacatattactcagtctccgtgaagggccgcttcaccatctccagagacaattccaagaacacggtgtttctacaaatgaacagcctgagagccgaggacacggccatatattactgtgtgaaagaaaggggccacagctggttcggggactggttcgacccctggggccagggaaccctggtcaccgtctcctca

SEQ ID NO: 289 - protein, 122 aa, synthetic construct
  REGION 1..122: note = synthetic
EVQLVESGGNLVQPGGSLRLSCAASGFTFSNYAMSWVRQAPGKGLEWVSGITGNGINTYYSVSVKGRFTISRDNSKNTVFLQMNSLRAEDTAIYYCVKERGHSWFGDWFDPWGQGTLVTVSS

SEQ ID NO: 290 - DNA, 24 bp, synthetic construct
  misc_feature 1..24: note = synthetic
  source 1..24: mol_type = other DNA
atcactggcaatggtattaataca

SEQ ID NO: 291 - protein, 8 aa, synthetic construct
  REGION 1..8: note = synthetic
ITGNGINT

SEQ ID NO: 292 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtggactttcggccaagggaccaaggtggaaatcaaa

SEQ ID NO: 293 - protein, 107 aa, synthetic construct
  REGION 1..107: note = synthetic
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPWTFGQGTKVEIK

SEQ ID NO: 294 - DNA, 27 bp, synthetic construct
  misc_feature 1..27: note = synthetic
  source 1..27: mol_type = other DNA
ctacagcataatagttacccgtggact

SEQ ID NO: 295 - DNA, 1350 bp, synthetic construct
  misc_feature 1..1350: note = synthetic
  source 1..1350: mol_type = other DNA
gaggtgcagctggtggagtctgggggaaacttggtacagccgggggggtccctgagactctcctgtgcagcctctggattcacctttagcaattatgccatgagttgggtccgccaggctccagggaagggactggagtgggtctcaggtatcactggcaatggtattaatacatattactcagtctccgtgaagggccgcttcaccatctccagagacaattccaagaacacggtgtttctacaaatgaacagcctgagagccgaggacacggccatatattactgtgtgaaagaaaggggccacagctggttcggggactggttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 296 - protein, 449 aa, synthetic construct
  REGION 1..449: note = synthetic
EVQLVESGGNLVQPGGSLRLSCAASGFTFSNYAMSWVRQAPGKGLEWVSGITGNGINTYYSVSVKGRFTISRDNSKNTVFLQMNSLRAEDTAIYYCVKERGHSWFGDWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 297 - DNA, 645 bp, synthetic construct
  misc_feature 1..645: note = synthetic
  source 1..645: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtggactttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 298 - protein, 214 aa, synthetic construct
  REGION 1..214: note = synthetic
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - DNA, 369 bp, synthetic construct
  misc_feature 1..369: note = synthetic
  source 1..369: mol_type = other DNA
caggtgcagctggtggagtctgggggaggcgtggtccagcctgggaggtccctgagactctcctgtgaagtctctggattcacatttagtaattatggcatgcactgggtccgccaggctccaggcaaggggctggagtgggtggcagttgtatcatatgatggaagtaataaatactatacagactccgtgaagggccgattcaccatctccagagacaattccaagaacacgctgtctctgcaaatgaatagcctgagagctgaagacacggctgtgtattactgtgcgaaagatcggggtatcactggcacctcgggcggtgtttttgatatctggggccaagggacaatggtcaccgtctcttca

SEQ ID NO: 300 - protein, 123 aa, synthetic construct
  REGION 1..123: note = synthetic
QVQLVESGGGVVQPGRSLRLSCEVSGFTFSNYGMHWVRQAPGKGLEWVAVVSYDGSNKYYTDSVKGRFTISRDNSKNTLSLQMNSLRAEDTAVYYCAKDRGITGTSGGVFDIWGQGTMVTVSS

SEQ ID NO: 301 - DNA, 24 bp, synthetic construct
  misc_feature 1..24: note = synthetic
  source 1..24: mol_type = other DNA
ggattcacatttagtaattatggc

SEQ ID NO: 302 - DNA, 24 bp, synthetic construct
  misc_feature 1..24: note = synthetic
  source 1..24: mol_type = other DNA
gtatcatatgatggaagtaataaa

SEQ ID NO: 303 - protein, 8 aa, synthetic construct
  REGION 1..8: note = synthetic
VSYDGSNK

SEQ ID NO: 304 - DNA, 48 bp, synthetic construct
  misc_feature 1..48: note = synthetic
  source 1..48: mol_type = other DNA
gcgaaagatcggggtatcactggcacctcgggcggtgtttttgatatc

SEQ ID NO: 305 - protein, 15 aa, synthetic construct
  REGION 1..15: note = synthetic
AKDRGITGTSGGVFD

SEQ ID NO: 306 - DNA, 321 bp, synthetic construct
  misc_feature 1..321: note = synthetic
  source 1..321: mol_type = other DNA
gccatccggatgacccagtctccatcctcattctctgcaactacaggagacagagtcaccatcacttgtcgggcgagtcagggtattagcagtcatttagcctggtatcagcaaaaaccagggaaagcccctaagttcctgatctatgttgcatccactttgcaaagtggggtcccatcaaggttcagcggcagtggatctgggacagatttcactctcaccatcagcagcctgcagtctgaagattttgcaacttattactgtcaacagtattatagttaccctccgacgttcggccaagggaccaaggtggaaatcaaa

SEQ ID NO: 307 - protein, 107 aa, synthetic construct
  REGION 1..107: note = synthetic
AIRMTQSPSSFSATTGDRVTITCRASQGISSHLAWYQQKPGKAPKFLIYVASTLQSGVPSRFSGSGSGTDFTLTISSLQSEDFATYYCQQYYSYPPTFGQGTKVEIK

SEQ ID NO: 308 - DNA, 18 bp, synthetic construct
  misc_feature 1..18: note = synthetic
  source 1..18: mol_type = other DNA
cagggtattagcagtcat

SEQ ID NO: 309 - protein, 6 aa, synthetic construct
  REGION 1..6: note = synthetic
QGISSH

SEQ ID NO: 310-311 - skipped (2 entries)

SEQ ID NO: 312 - DNA, 27 bp, synthetic construct
  misc_feature 1..27: note = synthetic
  source 1..27: mol_type = other DNA
caacagtattatagttaccctccgacg

SEQ ID NO: 313 - protein, 9 aa, synthetic construct
  REGION 1..9: note = synthetic
QQYYSYPPT

SEQ ID NO: 314 - DNA, 1353 bp, synthetic construct
  misc_feature 1..1353: note = synthetic
  source 1..1353: mol_type = other DNA
caggtgcagctggtggagtctgggggaggcgtggtccagcctgggaggtccctgagactctcctgtgaagtctctggattcacatttagtaattatggcatgcactgggtccgccaggctccaggcaaggggctggagtgggtggcagttgtatcatatgatggaagtaataaatactatacagactccgtgaagggccgattcaccatctccagagacaattccaagaacacgctgtctctgcaaatgaatagcctgagagctgaagacacggctgtgtattactgtgcgaaagatcggggtatcactggcacctcgggcggtgtttttgatatctggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 315 - protein, 450 aa, synthetic construct
  REGION 1..450: note = synthetic
QVQLVESGGGVVQPGRSLRLSCEVSGFTFSNYGMHWVRQAPGKGLEWVAVVSYDGSNKYYTDSVKGRFTISRDNSKNTLSLQMNSLRAEDTAVYYCAKDRGITGTSGGVFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 316 - DNA, 645 bp, synthetic construct
  misc_feature 1..645: note = synthetic
  source 1..645: mol_type = other DNA
gccatccggatgacccagtctccatcctcattctctgcaactacaggagacagagtcaccatcacttgtcgggcgagtcagggtattagcagtcatttagcctggtatcagcaaaaaccagggaaagcccctaagttcctgatctatgttgcatccactttgcaaagtggggtcccatcaaggttcagcggcagtggatctgggacagatttcactctcaccatcagcagcctgcagtctgaagattttgcaacttattactgtcaacagtattatagttaccctccgacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 317 - protein, 214 aa, synthetic construct
  REGION 1..214: note = synthetic
AIRMTQSPSSFSATTGDRVTITCRASQGISSHLAWYQQKPGKAPKFLIYVASTLQSGVPSRFSGSGSGTDFTLTISSLQSEDFATYYCQQYYSYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 318 - DNA, 342 bp, synthetic construct
  misc_feature 1..342: note = synthetic
  source 1..342: mol_type = other DNA
gaggtgcagctggtggagtcggggggaggcttggtccagcctggggggtccctgagactctcctgtgcagcctctggattcacgtctagtagatattggatgagttgggtccgccaggctccagggaaggggctggagtggctggccaacataaatcaagacggaagtgagaaatactatctggagtctctgaggggccgattcaccatctccagagacaacgccaagaactcactgtatctgcaaatgaacaacctgagagccgaggacacggctgtgtattactgtgcgagggatgaggaaggttactggggccagggaaccctggtcaccgtctcctca

SEQ ID NO: 319 - protein, 114 aa, synthetic construct
  REGION 1..114: note = synthetic
EVQLVESGGGLVQPGGSLRLSCAASGFTSSRYWMSWVRQAPGKGLEWLANINQDGSEKYYLESLRGRFTISRDNAKNSLYLQMNNLRAEDTAVYYCARDEEGYWGQGTLVTVSS

SEQ ID NO: 320 - DNA, 24 bp, synthetic construct
  misc_feature 1..24: note = synthetic
  source 1..24: mol_type = other DNA
ggattcacgtctagtagatattgg

SEQ ID NO: 321 - protein, 8 aa, synthetic construct
  REGION 1..8: note = synthetic
GFTSSRYW

SEQ ID NO: 322 - DNA, 24 bp, synthetic construct
  misc_feature 1..24: note = synthetic
  source 1..24: mol_type = other DNA
ataaatcaagacggaagtgagaaa

SEQ ID NO: 323 - protein, 8 aa, synthetic construct
  REGION 1..8: note = synthetic
INQDGSEK

SEQ ID NO: 324 - DNA, 21 bp, synthetic construct
  misc_feature 1..21: note = synthetic
  source 1..21: mol_type = other DNA
gcgagggatgaggaaggttac

SEQ ID NO: 325 - protein, 7 aa, synthetic construct
  REGION 1..7: note = synthetic
ARDEEGY

SEQ ID NO: 326 - DNA, 336 bp, synthetic construct
  misc_feature 1..336: note = synthetic
  source 1..336: mol_type = other DNA
gatgttgtgatgactcagtctctactctccctgtccgtcacccttggacagccggcctccatctcctgcaggtctagtcaaagcctcgtatacagtgatggggacacctacttgagttggtttcagcagaggccaggccaatctccacggcgcctaatttataaggtttctcaccgggactctggggtcccagacagattcagaggcagtggttcaggcactgatttcacactgaaaatcagcagggtggaggctgaggatgttgggatttattactgcatgcaaggtacacactggcctccgacgttcggccaagggaccaaggtggaaatcaaa

SEQ ID NO: 327 - protein, 112 aa, synthetic construct
  REGION 1..112: note = synthetic
DVVMTQSLLSLSVTLGQPASISCRSSQSLVYSDGDTYLSWFQQRPGQSPRRLIYKVSHRDSGVPDRFRGSGSGTDFTLKISRVEAEDVGIYYCMQGTHWPPTFGQGTKVEIK

SEQ ID NO: 328 - DNA, 33 bp, synthetic construct
  misc_feature 1..33: note = synthetic
  source 1..33: mol_type = other DNA
caaagcctcgtatacagtgatggggacacctac

SEQ ID NO: 329 - protein, 11 aa, synthetic construct
  REGION 1..11: note = synthetic
QSLVYSDGDTY

SEQ ID NO: 330-331 - skipped (2 entries)

SEQ ID NO: 332 - DNA, 27 bp, synthetic construct
  misc_feature 1..27: note = synthetic
  source 1..27: mol_type = other DNA
atgcaaggtacacactggcctccgacg

SEQ ID NO: 333 - protein, 9 aa, synthetic construct
  REGION 1..9: note = synthetic
MQGTHWPPT

SEQ ID NO: 334 - DNA, 1326 bp, synthetic construct
  misc_feature 1..1326: note = synthetic
  source 1..1326: mol_type = other DNA
gaggtgcagctggtggagtcggggggaggcttggtccagcctggggggtccctgagactctcctgtgcagcctctggattcacgtctagtagatattggatgagttgggtccgccaggctccagggaaggggctggagtggctggccaacataaatcaagacggaagtgagaaatactatctggagtctctgaggggccgattcaccatctccagagacaacgccaagaactcactgtatctgcaaatgaacaacctgagagccgaggacacggctgtgtattactgtgcgagggatgaggaaggttactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 335 - protein, 441 aa, synthetic construct
  REGION 1..441: note = synthetic
EVQLVESGGGLVQPGGSLRLSCAASGFTSSRYWMSWVRQAPGKGLEWLANINQDGSEKYYLESLRGRFTISRDNAKNSLYLQMNNLRAEDTAVYYCARDEEGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 336 - DNA, 660 bp, synthetic construct
  misc_feature 1..660: note = synthetic
  source 1..660: mol_type = other DNA
gatgttgtgatgactcagtctctactctccctgtccgtcacccttggacagccggcctccatctcctgcaggtctagtcaaagcctcgtatacagtgatggggacacctacttgagttggtttcagcagaggccaggccaatctccacggcgcctaatttataaggtttctcaccgggactctggggtcccagacagattcagaggcagtggttcaggcactgatttcacactgaaaatcagcagggtggaggctgaggatgttgggatttattactgcatgcaaggtacacactggcctccgacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 337 - protein, 219 aa, synthetic construct
  REGION 1..219: note = synthetic
DVVMTQSLLSLSVTLGQPASISCRSSQSLVYSDGDTYLSWFQQRPGQSPRRLIYKVSHRDSGVPDRFRGSGSGTDFTLKISRVEAEDVGIYYCMQGTHWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 338 - protein, 131 aa, synthetic construct
  REGION 1..131: note = Amino acids 1-103: Human ACVR1 extracellular domain (21-123 of accession number Q04771) Amino acids 104-131: Myc-myc-hexahistidine tag
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEEQKLISEEDLGGEQKLISEEDLHHHHHH

SEQ ID NO: 339 - protein, 336 aa, synthetic construct
  REGION 1..336: note = Amino acids 1-103: Human ACVR1 extracellular domain (21-123 of accession number Q04771) Amino acids 104-336: mFc
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 340 - protein, 131 aa, synthetic construct
  REGION 1..131: note = Amino acids 1-103: Mouse ACVR1 extracellular domain (21-123 of accession number P37172) Amino acids 104-131: Myc-myc-hexahistidine tag
VEDEKPKVNQKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEEQKLISEEDLGGEQKLISEEDLHHHHHH

SEQ ID NO: 341 - protein, 509 aa, synthetic construct
  REGION 1..509: note = Human ACVR1 Accession Q04771
MVDGVMILPVLIMIALPSPSMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACLLGVALRKFKRRNQERLNPRDVEYGTIEGLITTNVGDSTLADLLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKPAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

SEQ ID NO: 342 - protein, 509 aa, synthetic construct
  REGION 1..509: note = Mouse ACVR1 Accession P37172
MVDGVMILPVLMMMAFPSPSVEDEKPKVNQKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACILGVALRKFKRRNQERLNPRDVEYGTIEGLITTNVGDSTLAELLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKSAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

(19) United States
(12) Patent Application Publication
(10) Pub. No.: US 2024/0190974 A1
(43) Pub. Date: Jun. 13, 2024

(54) ANTI-ACVR1 ANTIBODIES AND USES THEREOF

(71) Applicant: Regeneron Pharmaceuticals, Inc., Tarrytown, NY (US)

(72) Inventors: Vincent J. IDONE, Ridgefield, CT (US); Sarah J. HATSELL, Nyack, NY (US); Aris N. ECONOMIDES, Tarrytown, NY (US)

(21) Appl. No.: 18/584,551

(22) Filed: Feb. 22, 2024

Related U.S. Application Data
(62) Division of application No. 17/172,856, filed on Feb. 10, 2021, now Pat. No. 11,945,872.
(60) Provisional application No. 62/975,047, filed on Feb. 11, 2020.
(60) Provisional application No. 63/030,131, filed on May 26, 2020.

(51) Int. Cl. C07K 16/28; A61K 39/00; A61K 39/395; A61K 45/06; A61P 7/06; A61P 19/00

(52) U.S. Cl. CPC C07K 16/2863; A61K 39/3955; A61K 45/06; A61P 7/06; A61P 19/00; A61K 2039/505; C07K 2317/21; C07K 2317/33; C07K 2317/565; C07K 2317/76; C07K 2317/92

(57) ABSTRACT

The present invention provides monoclonal antibodies that bind to the Activin A type I receptor (ACVR1) protein, and methods of use thereof. In various embodiments of the invention, the antibodies are fully human antibodies that bind to ACVR1. In some embodiments, the antibodies of the invention are useful for inhibiting ACVR1-mediated bone morphogenetic protein (BMP) signal transduction, thus providing a means of treating or preventing a disease, disorder or condition associated with ACVR1.

### CROSS REFERENCES TO RELATED APPLICATIONS

This application claims the benefit under 35 U.S.C. § 119(e) of U.S. provisional application No. 62/975,047, filed Feb. 11, 2020, and U.S. provisional application No. 63/030,131, filed May 26, 2020, the entire contents of each of which are incorporated herein by reference.

### SEQUENCE LISTING

The instant application includes a Sequence Listing which has been submitted electronically in XML format and is hereby incorporated by reference in its entirety. Said XML copy, created on Feb. 21, 2024 is named Sequence-Listing-40848-0101USD1 and is 467,032 bytes in size.

### FIELD OF THE INVENTION

The present invention is related to antibodies and antigen-binding fragments of antibodies that specifically bind to Activin A receptor type 1 (ACVR1) and/or ACVR1 mutant proteins, and therapeutic and diagnostic methods of using those antibodies.

### BACKGROUND OF THE INVENTION

Activin A receptor type 1 (ACVR1; also known as ActR1; or Activin receptor-like kinase 2; ALK2) is a single-pass transmembrane receptor, and a member of the type I Bone Morphogenic Protein (BMP) receptor of the TGF-β receptor super family. Upon ligand binding, ACVR1 together with a type II receptor initiates a downstream signaling cascade leading to activation of receptor specific R-SMAD protein (SMAD1, SMAD5, or SMAD8) which then associates with SMAD4, leading to transcriptional regulation of genes (Massague 1998, Massaque et al. 2005).

Mutations in ACVR1 gene which encodes the BMP type I receptor ALK2, also known as ACVR1 protein, may cause fibroplasia ossificans progressiva (FOP), a rare disorder leading to progressive ectopic bone formation in soft tissues with severe impairment of body movements because of extraskeletal bone bridges. ACVR1 mutations responsible for FOP cause dysregulation of SMAD-dependent downstream signaling and confer to the mutated receptor the ability to respond to noncanonical ligand, Activin A, triggering ectopic bone formation. Gain of function mutations in the gene encoding ACVR1 lead to debilitating disorders of extra-skeletal (heterotopic) ossification in humans such as FOP. For example, the typical FOP patient may have the amino acid arginine substituted for the amino acid histidine at position 206 of ACVR1 protein. This causes a change in glycine-serine activation domain of the protein, which converts an Acvr1:Activin A:Acvr2 non-signaling complex into a signaling complex. The result of the Activin neo-function is that Fibro-adipogenic progenitor (FAP) cells initiate endochondral ossification. Atypical mutations involving other residues may work similarly, resulting in the ACVR1 protein to be stuck in its active conformation despite no BMP being present. Mutations in the ACVR1 gene may also be linked to diffuse intrinsic pontine glioma (DIPG).

The liver expression of the key iron regulator hepcidin is controlled by the bone morphogenic protein (BMP)/SMAD pathway. BMP signaling requires the ligand (e.g., BMP7, BMP6, or BMP2), type I (e.g., ACVR1), type II receptors (e.g., ACVR2 or BMPR2), and coreceptor hemojuvelin (HJV) to phosphorylate SMAD proteins. BMP6 mediated activation of ACVR1 directly activates transcription of Hamp, the gene that encodes hepcidin. Hepcidin is a negative regulator of iron levels by causing internalization of ferroportin (sic40a1), the only known iron exporter. Inhibition of the BMP6-ACVR1 signaling cascade leads to decreased Hamp transcription, resulting in decreased circulating levels of hepcidin. A reduction of circulating hepcidin results in increased ferroportin levels, which allows increased uptake of iron from the small intestines, thereby increasing circulating iron levels.

Monoclonal antibodies to ACVR1 are described in Katagiri et al., US Patent/Publication Nos. 10428148, 20180118835, and in WO 2019172165.

Fully human antibodies that specifically bind to ACVR1 protein, a fragment thereof, or a mutant thereof with high affinity and that inhibit ACVR1-mediated bone morphogenetic protein (BMP) signal transduction could be important in the prevention and treatment of, e.g., heterotopic ossification, ectopic ossification, bone dysplasia, anemia, or diffuse intrinsic pontine glioma.

### BRIEF SUMMARY OF THE INVENTION

The present invention provides antibodies and antigen-binding fragments thereof that specifically bind to an Activin A receptor type 1 (ACVR1) protein and inhibit ACVR1-mediated BMP signal transduction. In certain embodiments, the anti-ACVR1 antibodies are fully human antibodies that bind to ACVR1 with high affinity and block ACVR1 or destabilize the activated conformation. The antibodies of the present invention are useful, inter alia, for deactivating or decreasing the activity of ACVR1 protein. In certain embodiments, the antibodies are useful in preventing, treating or ameliorating at least one symptom or indication of a ACVR 1-associated disease or disorder in a subject. In certain embodiments, the antibodies may be administered prophylactically or therapeutically to a subject having or at risk of having a ACVR1-associated disease or disorder. In specific embodiments, the antibodies are used in the prevention and treatment of heterotopic ossification, ectopic ossification, bone dysplasia, anemia, or certain cancers, including brain tumors when administered to a subject in need thereof.

In some embodiments, the antibodies of the invention bind to an ACVR1 protein and/or a mutant thereof. Further, the antibodies disclosed herein bind to an ACVR1 protein or a mutant thereof with high affinity. ACVR1 proteins used in the present invention include ACVR1 proteins which may be derived from a mammal such as a human or a mouse. For example, the full-length amino acid sequence of human ACVR1 is available with reference to UniProtKB Accession No. Q04771 (SEQ ID NO: 341).

The ACVR1 protein may include a signal peptide occurring at positions 1-20 of ACVR1 protein, for example, of accession number Q04771 (SEQ ID NO: 341). The mature ACVR1 protein may include amino acids 21-509, for example, of accession number Q04771 (SEQ ID NO: 341). The ACVR1 protein may include an extracellular domain at amino acids 21-123 of, for example, accession number Q04771 (SEQ ID NO: 341). The ACVR1 protein may include a transmembrane domain at amino acids 124-146 of, for example, accession number Q04771 (SEQ ID NO: 341). The ACVR1 protein may include a protein kinase domain within positions 208-502, for example, of accession number Q04771 (SEQ ID NO: 341). The ACVR protein may include glycosylation at amino acid position 102 comprising an N-linked (GlcNAc . . . ) asparagine, for example, of accession number Q04771 (SEQ ID NO: 341). The ACVR protein may include a modified residue for example, such as phosphoserine at position 501, for example, of accession number Q04771 (SEQ ID NO: 341).

Mutations in the ACVR1 gene may be a responsible for various diseases including FOP. The ACVR1 protein may be a mutant ACVR1 protein having amino acid substitutions which may be found in various familial and sporadic FOP cases. The human ACVR1 protein may comprise various mutations, including but not limited to L196P (mutation that substitutes leucine at position 196 by proline), delP197_F198insL (mutation that deletes proline at position 197 and phenylalanine at position 198 and inserts leucine), R202I (mutation that substitutes arginine at position 202 by isoleucine), R206H (mutation that substitutes arginine at position 206 by histidine), Q207E (mutation that substitutes glutamine at position 207 by glutamic acid), R258S (mutation that substitutes arginine at position 258 by serine), R258G (mutation that substitutes arginine at position 258 by glycine), G325A (mutation that substitutes glycine at position 325 by alanine), G328E (mutation that substitutes glycine at position 328 by glutamic acid), G328R (mutation that substitutes glycine at position 328 by arginine), G328W (mutation that substitutes glycine at position 328 by tryptophan), G356D (mutation that substitutes glycine at position 356 by aspartic acid), and R375P (mutation that substitutes arginine at position 375 by proline) of SEQ ID NO: 341.

As another example, the full-length amino acid sequence of mouse ACVR1 protein is available with reference to Accession No. P37172 (SEQ ID NO: 342).

The antibodies of the invention can be full-length (for example, an IgG1 or IgG4 antibody) or may comprise only an antigen-binding portion (for example, a Fab, F(ab′)₂ or scFv fragment), and may be modified to affect functionality, e.g., to increase persistence in the host or to eliminate residual effector functions (Reddy et al., 2000, J. Immunol. 164: 1925-1933). In certain embodiments, the antibodies may be bispecific.

In a first aspect, the present invention provides isolated recombinant monoclonal antibodies or antigen-binding fragments thereof that bind specifically to an ACVR1 protein. In some embodiments, the antibodies are fully human monoclonal antibodies.

Exemplary anti-ACVR1 antibodies of the present invention are listed in Tables 1 and 2 herein. Table 1 sets forth the amino acid sequence identifiers of the heavy chain variable regions (HCVRs), light chain variable regions (LCVRs), heavy chain complementarity determining regions (HCDRs) (HCDR1, HCDR2 and HCDR3), and light chain complementarity determining regions (LCDRs) (LCDR1, LCDR2 and LCDR3) of exemplary antibodies. Table 2 sets forth the nucleic acid sequence identifiers of the HCVRs, LCVRs, HCDR1, HCDR2 HCDR3, LCDR1, LCDR2 and LCDR3 of the exemplary antibodies.

The present invention provides antibodies, or antigen-binding fragments thereof, comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

The present invention also provides antibodies, or antigen-binding fragments thereof, comprising an LCVR comprising an amino acid sequence selected from any of the LCVR amino acid sequences listed in Table 1, or a substantially similar sequence thereof having at least 90%, at least 95%, at least 98% or at least 99% sequence identity thereto.

The present invention also provides antibodies, or antigen-binding fragments thereof, comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, the present invention provides antibodies, or antigen-binding fragments thereof, comprising an HCVR/LCVR amino acid sequence pair contained within any of the exemplary anti-ACVR1 antibodies listed in Table 1. In certain embodiments, the anti-ACVR1 antibodies of the invention comprise an HCVR/LCVR amino acid sequence pair selected from one of SEQ ID NOs: 2/10 (e.g., mAb27396), 22/30 (e.g., mAb27241), 22/72 (e.g., mAb27245), 42/48 (e.g., mAb27242), 58/62 (e.g., mAb27243), 76/84 (e.g., mAb27247), 96/104 (e.g., mAb27404), 116/119 (e.g., mAb27405), 128/136 (e.g., mAb27400), 203/211 (e.g., mAb29226), 273/277 (e.g., mAb29257), and 300/307 (e.g., mAb29266).

The present invention also provides antibodies, or antigen-binding fragments thereof, comprising a HCVR and a LCVR, said HCVR comprising an amino acid sequence listed in Table 1 having no more than twelve amino acid substitutions, and/or said LCVR comprising an amino acid sequence listed in Table 1 having no more than ten amino acid substitutions. For example, the present invention provides antibodies or antigen-binding fragments thereof comprising a HCVR and a LCVR, said HCVR comprising an amino acid sequence listed in Table 1, said amino acid sequence having one, two, three, four, five, six, seven, eight, nine, ten, eleven or twelve amino acid substitutions. In another example, the present invention provides antibodies or antigen-binding fragments thereof comprising a HCVR and a LCVR, said LCVR comprising an amino acid sequence listed in Table 1, said amino acid sequence having one, two, three, four, five, six, seven, eight, nine or ten amino acid substitutions. In one embodiment, the present invention provides anti-ACVR1 antibodies or antigen-binding fragments thereof comprising a HCVR and a LCVR, said HCVR comprising an amino acid sequence listed in Table 1, said amino acid sequence having at least one amino acid substitution, and/or said LCVR comprising an amino acid sequence listed in Table 1, said amino acid sequence having at least one amino acid substitution.

The present invention also provides antibodies, or antigen-binding fragments thereof, comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity.

The present invention also provides antibodies, or antigen-binding fragments thereof, comprising an HCDR3 and an LCDR3 amino acid sequence pair (HCDR3/LCDR3) comprising any of the HCDR3 amino acid sequences listed in Table 1 paired with any of the LCDR3 amino acid sequences listed in Table 1. According to certain embodiments, the present invention provides antibodies, or antigen-binding fragments thereof, comprising an HCDR3/LCDR3 amino acid sequence pair contained within any of the exemplary anti-ACVR1 antibodies listed in Table 1. In certain embodiments, the HCDR3/LCDR3 amino acid sequence pair is selected from the group consisting of SEQ ID NOs:28/36 (e.g., mAb27242), 60/66 (e.g., mAb27243), 82/90 (e.g., mAb27247), 8/16 (e.g., mAb27396), 102/110 (e.g., mAb27405), 28/66 (e.g., mAb27245), 134/142 (e.g., mAb27400), 209/217 (e.g., mAb29226), 261/283 (e.g., mAb29257), and 305/313 (e.g., mAb29266).

The present invention also provides antibodies, or antigen-binding fragments thereof, comprising a HCVR and a LCVR, said HCVR comprising HCDR1 comprising an amino acid sequence differing from an amino acid sequence listed in Table 1 by 1 amino acid, HCDR2 comprising an amino acid sequence differing from an amino acid sequence listed in Table 1 by 1 amino acid, and HCDR3 comprising an amino acid sequence differing from an amino acid sequence listed in Table 1 by 1 amino acid. In certain embodiments, the present invention provides antibodies, or antigen-binding fragments thereof, comprising a HCVR and a LCVR, said LCVR comprising LCDR1 comprising an amino acid sequence differing from an amino acid sequence listed in Table 1 by 1 amino acid, LCDR2 comprising an amino acid sequence differing from an amino acid sequence listed in Table 1 by 1 amino acid, and LCDR3 comprising an amino acid sequence differing from an amino acid sequence listed in Table 1 by 1 amino acid. For example, the present invention provides antibodies, or antigen-binding fragments thereof, comprising a HCVR and a LCVR, said HCVR comprising HCDR1 comprising an amino acid sequence of SEQ ID NO: 24 or 44 or an amino acid sequence differing from SEQ ID NO: 24 or 44 by 1 amino acid, HCDR2 comprising an amino acid sequence of SEQ ID NO: 46 or an amino acid sequence differing from SEQ ID NO: 46 by 1 amino acid, and HCDR3 comprising an amino acid sequence of SEQ ID NO: 28 or 60 or an amino acid sequence differing from SEQ ID NO: 28 or 60 by 1 amino acid. In another exemplary embodiment, the present invention provides antibodies, or antigen-binding fragments thereof, comprising a HCVR and a LCVR, said LCVR comprising LCDR1 comprising an amino acid sequence of SEQ ID NO : 50 or an amino acid sequence differing from SEQ ID NO: 50 by 1 amino acid, LCDR2 comprising an amino acid sequence of SEQ ID NO: 52 or 64 or an amino acid sequence differing from SEQ ID NO: 52 or 64 by 1 amino acid, and LCDR3 comprising an amino acid sequence of SEQ ID NO: 36 or 66 or an amino acid sequence differing from SEQ ID NO: 36 or 66 by 1 amino acid.

The present invention also provides antibodies, or antigen-binding fragments thereof, comprising a set of six CDRs (i.e., HCDR1-HCDR2-HCDR3-LCDR1-LCDR2-LCDR3) contained within any of the exemplary antibodies listed in Table 1. In certain embodiments, the HCDR1-HCDR2-HCDR3-LCDR1-LCDR2-LCDR3 amino acid sequence set is selected from the group consisting of SEQ ID NOs: 44-46-28-50-52-36 (e.g., mAb27242), 24-46-60-50-64-66 (e.g., mAb27243), 78-80-82-86-88-90 (e.g., mAb27247), 4-6-8-12-14-16 (e.g., mAb27396), 98-100-102-106-122-110 (e.g., mAb27405), 98-100-102-106-108-110(e.g., mAb27404), 24-26-28-50-64-66 (e.g., mAb27245), 24-26-28-32-34-36 (e.g., mAb27241), 130-132-134-138-140-142 (e.g., mAb27400), 205-207-209-213-215-217 (e.g., mAb29226), 257-275-261-279-281-283 (e.g., mAb29257), and 4-303-305-309-311-313 (e.g., mAb29266).

In a related embodiment, the present invention provides antibodies, or antigen-binding fragments thereof, comprising a set of six CDRs (i.e., HCDR1-HCDR2-HCDR3-LCDR1-LCDR2-LCDR3) contained within an HCVR/LCVR amino acid sequence pair as defined by any of the exemplary antibodies listed in Table 1. For example, the present invention includes antibodies, or antigen-binding fragments thereof, comprising the HCDR1-HCDR2-HCDR3-LCDR1-LCDR2-LCDR3 amino acid sequences set contained within an HCVR/LCVR amino acid sequence pair selected from the group consisting of SEQ ID NOs: 2/10 (e.g., mAb27396), 22/30 (e.g., mAb27241), 22/72 (e.g., mAb27245), 42/48 (e.g., mAb27242), 58/62 (e.g., mAb27243), 76/84 (e.g., mAb27247), 96/104 (e.g., mAb27404), 116/119 (e.g., mAb27405), 128/136 (e.g., mAb27400), 203/211 (e.g., mAb29226), 273/277 (e.g., mAb29257), and 300/307 (e.g., mAb29266).

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments, the present invention includes an antibody or antigen-binding fragment thereof that binds specifically to ACVR1, wherein the antibody or antigen-binding fragment thereof comprises three heavy chain complementarity determining regions (CDRs) (HCDR1, HCDR2 and HCDR3) contained within a heavy chain variable region (HCVR) and three light chain CDRs (LCDR1, LCDR2 and LCDR3) contained within a light chain variable region (LCVR), wherein the HCVR comprises: (i) an amino acid sequence selected from the group consisting of SEQ ID NOs: 2, 22, 42, 58, 76, 96, 116, 128, 148, 166, 186, 203, 223, 241, 255, 273, 289, 300, and 319; (ii) an amino acid sequence having at least 90% identity to the amino acid sequence selected from the group consisting of SEQ ID NOs: 2, 22, 42, 58, 76, 96, 116, 128, 148, 166, 186, 203, 223, 241, 255, 273, 289, 300, and 319; (iii) an amino acid sequence having at least 95% identity to the amino acid sequence selected from the group consisting of SEQ ID NOs: 2, 22, 42, 58, 76, 96, 116, 128, 148, 166, 186, 203, 223, 241, 255, 273, 289, 300, and 319; or (iv) an amino acid sequence selected from the group consisting of SEQ ID NOs: 2, 22, 42, 58, 76, 96, 116, 128, 148, 166, 186, 203, 223, 241, 255, 273, 289, 300, and 319, said amino acid sequence having no more than 12 amino acid substitutions; and the LCVR comprises: (a) an amino acid sequence selected from the group consisting of SEQ ID NOs: 10, 30, 48, 62, 72, 84, 104, 119, 136, 156, 174, 193, 211, 231, 245, 263, 277, 293, 307, and 327; (b) an amino acid sequence having at least 90% identity to the amino acid sequence selected from the group consisting of SEQ ID NOs: 10, 30, 48, 62, 72, 84, 104, 119, 136, 156, 174, 193, 211, 231, 245, 263, 277, 293, 307, and 327; (c) an amino acid sequence having at least 95% identity to the amino acid sequence selected from the group consisting of SEQ ID NOs: 10, 30, 48, 62, 72, 84, 104, 119, 136, 156, 174, 193, 211, 231, 245, 263, 277, 293, 307, and 327; or (d) an amino acid sequence selected from the group consisting of SEQ ID NOs: 10, 30, 48, 62, 72, 84, 104, 119, 136, 156, 174, 193, 211, 231, 245, 263, 277, 293, 307, and 327, said amino acid sequence having no more than 10 amino acid substitutions.

In certain preferred embodiments, the present invention includes antibodies that bind specifically to ACVR1 in an antagonist manner, i.e., decrease or block ACVR1 binding and/or activity.

The present invention includes anti-ACVR1 antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

In certain embodiments, the present invention provides antibodies and antigen-binding fragments thereof that exhibit pH-dependent binding to ACVR1. For example, the present invention includes antibodies and antigen-binding fragment thereof that bind ACVR1 with higher affinity at neutral pH than at acidic pH (i.e., reduced binding at acidic pH).

The present invention also provides for antibodies and antigen-binding fragments thereof that compete for specific binding to ACVR1 with an antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

The present invention also provides antibodies and antigen-binding fragments thereof that cross-compete for binding to ACVR1 with a reference antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

The present invention also provides antibodies and antigen-binding fragments thereof that bind to the same epitope as a reference antibody or antigen-binding fragment thereof comprising three CDRs of a HCVR and three CDRs of a LCVR, wherein the HCVR and LCVR each has an amino acid sequence selected from the HCVR and LCVR sequences listed in Table 1.

The present invention also provides isolated antibodies and antigen-binding fragments thereof that inhibit ligand-induced signaling by BMP7, Activin A or other TGFBeta family ligand forming a signaling complex with an Activin type II receptor. In some embodiments, the antibody or antigen-binding fragment thereof prevents ACVR1 from forming signaling complex with an Activin type II receptor. The present invention provides isolated antibodies and antigen-binding fragments thereof that may bind to the same epitope on ACVR1 as BMP7 or Activin A or an Activin type II receptor or may bind to a different epitope on ACVR1 as BMP7 or Activin A or an Activin type II receptor.

In certain embodiments, the antibodies or antigen-binding fragments of the present invention are bispecific comprising a first binding specificity to a first epitope of ACVR1 and a second binding specificity to a second epitope of ACVR1 wherein the first and second epitopes are distinct and non-overlapping.

In certain embodiments, the present invention provides an isolated antibody or antigen-binding fragment thereof that has one or more of the following characteristics:
(a) is a fully human monoclonal antibody;
(b) binds to human ACVR1 extracellular domain fused to an Fc (e.g., SEQ ID NO: 339) at 25° C. with a dissociation constant (K_{D}) of less than 60 nM, less than 12 nM, less than less than 2 nM, less than 1 nM, or less than 0.5 nM as measured in a surface plasmon resonance assay;
(c) binds to human ACVR1 extracellular domain fused to mFc (SEQ ID NO: 339) at 37° C. with a dissociation constant (K_{D}) of less than 150 nM, less than 15 nM, less than less than 5 nM, less than 1.5 nM, or less than 1 nM as measured in a surface plasmon resonance assay;
(d) binds to human ACVR1 extracellular domain fused to myc-myc-hexahistag (e.g., SEQ ID NO: 338) at 25° C. with a K_{D} of less than 300 nM, less than 150 nM, less than 25 nM, less than 10 nM, less than 5 nM, less than 3 nM or less than 2 nM as measured in a surface plasmon resonance assay;
(e) binds to human ACVR1 extracellular domain fused to myc-myc-hexahistag (e.g., SEQ ID NO: 338) at 37° C. with a K_{D} of less than 500 nM, less than 50 nM, less than 25 nM, less than 10 nM, as measured in a surface plasmon resonance assay; (f) does not bind mouse ACVR1 or binds to mouse ACVR1 extracellular domain fused to myc-myc-hexahistag (e.g., SEQ ID NO: 340) at 25° C. with a K_{D} of greater than 500 nM, as measured in a surface plasmon resonance assay; (g) does not bind mouse ACVR1 or binds to mouse ACVR1 extracellular domain fused to myc-myc-hexahistag (e.g., SEQ ID NO: 340) at 37° C. with a K_{D} of greater than 500 nM, as measured in a surface plasmon resonance assay;
(k) binds to cells expressing human ACVR1 protein or human ACVR (R206H) protein; (I) inhibits activation of cells expressing human ACVR1(R206H) by human Activin A with a IC₅₀ of less than 25 nM, as measured in a cell-based bioassay;
(m) inhibits activation of cells expressing human ACVR1(R206H) by human BMP7 with a IC₅₀ of less than 20 nM, less than 5 nM, less than 3 nM, or less than 1 nM, or less than as measured in a cell-based bioassay;
(m) significantly decreases serum hepcidin when administered to mice expressing human ACVR1 in place of mouse allele; (n) significantly increases serum iron levels when administered to mice expressing human ACVR1 in place of mouse allele; and/or (o) inhibits wild-type ACVR1 signaling when administered to mice expressing human ACVR1 in place of mouse allele; and
(o) comprises a HCVR comprising an amino acid sequence selected from the group consisting of HCVR sequence listed in Table 1 and a LCVR comprising an amino acid sequence selected from the group consisting of LCVR sequences listed in Table 1.

In a second aspect, the present invention provides nucleic acid molecules encoding anti-ACVR1 antibodies or portions thereof. For example, the present invention provides nucleic acid molecules encoding any of the HCVR amino acid sequences listed in Table 1; in certain embodiments the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto.

The present invention also provides nucleic acid molecules encoding any of the LCVR amino acid sequences listed in Table 1; in certain embodiments the nucleic acid molecule comprises a polynucleotide sequence selected from any of the LCVR nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR1 amino acid sequences listed in Table 1; in certain embodiments the nucleic acid molecule comprises a polynucleotide sequence selected from any of the HCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR2 amino acid sequences listed in Table 1; in certain embodiments the nucleic acid molecule comprises a polynucleotide sequence selected from any of the HCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR3 amino acid sequences listed in Table 1; in certain embodiments the nucleic acid molecule comprises a polynucleotide sequence selected from any of the HCDR3 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR1 amino acid sequences listed in Table 1; in certain embodiments the nucleic acid molecule comprises a polynucleotide sequence selected from any of the LCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR2 amino acid sequences listed in Table 1; in certain embodiments the nucleic acid molecule comprises a polynucleotide sequence selected from any of the LCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR3 amino acid sequences listed in Table 1; in certain embodiments the nucleic acid molecule comprises a polynucleotide sequence selected from any of the LCDR3 nucleic acid sequences listed in Table 2, or a substantially similar sequence thereof having at least 90%, at least 95%, at least 98% or at least 99% sequence identity thereto.

The present invention also provides nucleic acid molecules encoding an HCVR, wherein the HCVR comprises a set of three CDRs (i.e., HCDR1-HCDR2-HCDR3), wherein the HCDR1-HCDR2-HCDR3 amino acid sequence set is as defined by any of the exemplary antibodies listed in Table 1.

The present invention also provides nucleic acid molecules encoding an LCVR, wherein the LCVR comprises a set of three CDRs (i.e., LCDR1-LCDR2-LCDR3), wherein the LCDR1-LCDR2-LCDR3 amino acid sequence set is as defined by any of the exemplary antibodies listed in Table 1.

The present invention also provides nucleic acid molecules encoding both an HCVR and an LCVR, wherein the HCVR comprises an amino acid sequence of any of the HCVR amino acid sequences listed in Table 1, and wherein the LCVR comprises an amino acid sequence of any of the LCVR amino acid sequences listed in Table 1. In certain embodiments, the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto, and a polynucleotide sequence selected from any of the LCVR nucleic acid sequences listed in Table 1, or a substantially similar sequence thereof having at least 90%, at least 95%, at least 98% or at least 99% sequence identity thereto. In certain embodiments according to this aspect of the invention, the nucleic acid molecule encodes an HCVR and LCVR, wherein the HCVR and LCVR are both derived from the same anti-ACVR1 antibody listed in Table 1.

In a related aspect, the present invention provides recombinant expression vectors capable of expressing a polypeptide comprising a heavy and/or light chain variable region of an antibody. For example, the present invention includes recombinant expression vectors comprising any of the nucleic acid molecules mentioned above, i.e., nucleic acid molecules encoding any of the HCVR, LCVR, and/or CDR sequences as set forth in Table 2. In certain embodiments, the present invention provides expression vectors comprising: (a) a nucleic acid molecule comprising a nucleic acid sequence encoding a HCVR of an antibody that binds ACVR1, wherein the HCVR comprises an amino acid sequence selected from the group consisting of sequences listed in Table 1; and/or (b) a nucleic acid molecule comprising a nucleic acid sequence encoding a LCVR of an antibody that binds ACVR1, wherein the LCVR comprises an amino acid sequence selected from the group consisting of sequences listed in Table 1. Also included within the scope of the present invention are host cells into which such vectors have been introduced, as well as methods of producing the antibodies or portions thereof by culturing the host cells under conditions permitting production of the antibodies or antibody fragments, and recovering the antibodies and antibody fragments so produced. In certain embodiments, the host cells comprise a mammalian cell or a prokaryotic cell. In certain embodiments, the host cell is a Chinese Hamster Ovary (CHO) cell or an Escherichia coli (E. coli) cell. In certain embodiments, the present invention provides methods of producing an antibody or antigen-binding fragment thereof of the invention, the methods comprising introducing into a host cell an expression vector comprising a nucleic acid sequence encoding a HCVR and/or LCVR of an antibody or antigen-binding fragment thereof of the invention operably linked to a promoter; culturing the host cell under conditions favorable for expression of the nucleic acid sequence; and isolating the antibody or antigen-binding fragment thereof from the culture medium and/or host cell. The isolated antibody or antigen-binding fragment thereof may be purified using any of the methods known in prior art.

In a third aspect, the invention provides a pharmaceutical composition comprising a therapeutically effective amount of at least one recombinant monoclonal antibody or antigen-binding fragment thereof which specifically binds ACVR1 and a pharmaceutically acceptable carrier. In a related aspect, the invention features a composition which is a combination of an anti-ACVR1 antibody and a second therapeutic agent. In one embodiment, the second therapeutic agent is any agent that is advantageously combined with an anti-ACVR1 antibody.

Exemplary agents that may be advantageously combined with an anti-ACVR1 antibody include, without limitation, other agents that bind and/or activate ACVR1 activity (including other antibodies or antigen-binding fragments thereof, etc.) and/or agents which do not directly bind ACVR1 but nonetheless treat or ameliorate at least one symptom or indication of a ACVR1-associated disease or disorder (disclosed elsewhere herein). Additional combination therapies and co-formulations involving the anti-ACVR1 antibodies of the present invention are disclosed elsewhere herein.

In a fourth aspect, the invention provides therapeutic methods for treating a disease or disorder associated with ACVR1 in a subject using an anti-ACVR1 antibody or antigen-binding portion of an antibody of the invention, wherein the therapeutic methods comprise administering a therapeutically effective amount of a pharmaceutical composition comprising an antibody or antigen-binding fragment of an antibody of the invention to the subject in need thereof. The disorder treated is any disease or condition which is improved, ameliorated, inhibited or prevented by potentiation of ACVR1 activity (e.g., anemia, heterotopic ossification, ectopic ossification, bone dysplasia, or diffuse intrinsic pontine glioma). In certain embodiments, the invention provides methods to prevent, or treat a ACVR1-associated disease or disorder comprising administering a therapeutically effective amount of an anti-ACVR1 antibody or antigen-binding fragment thereof of the invention to a subject in need thereof. In some embodiments, the antibody or antigen-binding fragment thereof may be administered prophylactically or therapeutically to a subject having or at risk of having a ACVR1-associated disease or disorder. In certain embodiments, the antibody or antigen-binding fragment thereof the invention is administered in combination with a second therapeutic agent to the subject in need thereof.

The second therapeutic agent may be selected from the group consisting of an anti-Activin A antibody or antigen-binding fragment thereof, anti-BMP7 antibody or antigen binding fragment thereof, anti-ACVR2 antibody or antigen-binding fragment thereof, anti-inflammatory drugs, steroids, bisphosphonates, muscle relaxants, or retinoic acid receptor (RAR) gamma agonists, a lifestyle modification, a dietary supplement and any other drug or therapy known in the art. In certain embodiments, the second therapeutic agent may be an agent that helps to counteract or reduce any possible side effect(s) associated with an antibody or antigen-binding fragment thereof of the invention, if such side effect(s) should occur. The antibody or fragment thereof may be administered subcutaneously, intravenously, intradermally, intraperitoneally, orally, intramuscularly, or intracerebroventricularly. The antibody or fragment thereof may be administered at a dose of about 0.1 mg/kg of body weight to about 100 mg/kg of body weight of the subject. In certain embodiments, an antibody of the present invention may be administered at one or more doses comprising between 10mg to 600mg.

The present invention also includes use of an anti-ACVR1 antibody or antigen-binding fragment thereof of the invention in the manufacture of a medicament for the treatment of a disease or disorder that would benefit from the activation of ACVR1 binding and/or activity.

Other embodiments will become apparent from a review of the ensuing detailed description.

### BRIEF DESCRIPTION OF THE FIGURES

FIG. 1A shows a graph of total heterotopic ossification (HO) volume by microCT over a period of 13 weeks after surgery in an in vivo post-traumatic HO model in WT mice. Mice were administered either an isotype control antibody (circles, n=12), ALK3-Fc (squares, n=12) or an Acvr1 antibody mAb27242 (triangles, n=12) starting concurrently with induction of injury. HO volume was measured by μCT 3, 6, 9 and 13 weeks post injury. Acvr1 blocking antibodies significantly attenuated total HO compared to isotype control by 9 weeks post-surgery (p<0.05).

FIG. 1B shows a graph of attached heterotopic ossification (HO) volume by microCT over a period of 13 weeks after surgery in an in vivo post-traumatic HO model in WT mice. Mice were administered either an isotype control antibody (circles, n=12), ALK3-Fc (squares, n=12) or an Acvr1 antibody mAb27242 (triangles, n=12) starting concurrently with induction of injury. HO volume was measured by μCT 3, 6, 9 and 13 weeks post injury. Acvr1 blocking antibodies significantly attenuated attached HO compared to isotype control by 9 weeks post-surgery (p<0.05).

FIG. 1C shows a graph of unattached heterotopic ossification (HO) volume by microCT over a period of 13 weeks after surgery in an in vivo post-traumatic HO model in WT mice. Mice were administered either an isotype control antibody (circles, n=12), ALK3-Fc (squares, n=12) or an Acvr1 antibody mAb27242 (triangles, n=12) starting concurrently with induction of injury. HO volume was measured by μCT 3, 6, 9 and 13 weeks post injury. Acvr1 blocking antibodies significantly attenuated attached HO compared to isotype control by 13 weeks post-surgery (p<0.01).

FIGS. 2A-C show images of HO volume in the injured hindlimb in an in vivo post-traumatic HO model in WT mice as measured by total HO volume by microCT, attached HO (encircled by broken white lines) or unattached HO (encircled by short dashed white lines) 13 weeks post-surgery.

FIG. 2A shows a representative image of HO volume in the injured hindlimb in an in vivo post-traumatic HO model in WT mice as measured by total HO volume by microCT, attached HO (encircled by broken white lines) or unattached HO (encircled by short dashed white lines) 13 weeks post-surgery after receiving isotype control antibody.

FIG. 2B shows a representative image of HO volume in the injured hindlimb in an in vivo post-traumatic HO model in WT mice as measured by total HO volume by microCT, attached HO (encircled by broken white lines) or unattached HO (encircled by short dashed white lines) 13 weeks post-surgery after receiving Alk3-Fc. HO volumes by micro CT were significantly reduced after 13 weeks in mice receiving ALK3-Fc compared to mice receiving isotype control.

FIG. 2C shows a representative image of HO volume in the injured hindlimb in an in vivo post-traumatic HO model in WT mice as measured by total volume by microCT, attached HO (encircled by broken white lines) or unattached HO (encircled by short dashed white lines) 13 weeks post-surgery after receiving anti-ACVR antibody mAb27242. HO volumes by micro CT were significantly reduced after 13 weeks in mice receiving anti-ACVR antibody compared to mice receiving isotype control antibody.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, preferred methods and materials are now described. All publications, patents, and patent applications mentioned herein are incorporated herein by reference in their entirety.

### Definitions

The term “ACVR1”, also called “ALK2” refers to Activin A receptor type 1 (also known as Activin-like kinase 2). ACVR1 is a single-pass type I membrane protein. The full-length amino acid sequence of human ACVR1 is available with reference to UniProtKB Accession No. Q04771, as having 509 aa residues (SEQ ID NO: 341). The protein has an extracellular domain at amino acid residues 21-123, a transmembrane domain at amino acid positions 124-146, and a cytoplasmic domain at positions 147-509. On ligand binding, ACVR1 forms a receptor complex consisting of two type II and two type I transmembrane serine/threonine kinases. Type II receptors phosphorylate and activate type I receptors. Which autophosphorylate, then bind and activate SMAD transcriptional regulators. ACVR1 is a receptor for Activin.

The amino acid sequence of full-length human ACVR1 protein is exemplified by the amino acid sequence provided in UniProtKB/Swiss-Prot as accession number Q04771 (SEQ ID NO: 341). The full-length amino acid sequence of mouse ACVR1 protein is available with reference to Accession No. P37172 (SEQ ID NO: 342).

The term “ACVR1” includes recombinant ACVR1 protein or a fragment thereof. The term also encompasses ACVR1 protein or a fragment thereof coupled to, for example, a histidine tag, PADRE tag, mouse or human Fc, or a signal sequence (for example, SEQ ID NOs: 338-340).

The term “ACVR1” may include an ACVR1 protein or a fragment thereof comprising a mutation. For example, the mutation may be based on corresponding amino acid sequence or fragment thereof of human ACVR1 UniProtKB Accession No. Q04771, (SEQ ID NO: 341). For example, the ACVR1 protein or fragment thereof may comprise a mutation, including but not limited to L196P, delP197_F198insL, R202I, R206H, Q207E, R258S, R258G, G325A, G328E, G328R, G328W, G356D, and R375P of corresponding SEQ ID NO: 341.

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “V_{H}”) and a heavy chain constant region (comprised of domains C_{H}1, C_{H}2 and C_{H}3). Each light chain is comprised of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the invention, the FRs of the antibody (or antigen binding fragment thereof) may be identical to the human germline sequences, or may be naturally or artificially modified. An amino acid consensus sequence may be defined based on a side-by-side analysis of two or more CDRs.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The fully human anti-ACVR1 monoclonal antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic biological properties, reduced immunogenicity, etc. Antibodies and antigen-binding fragments obtained in this general manner are encompassed within the present invention.

The present invention also includes fully human anti-ACVR1 monoclonal antibodies comprising variants of any of the HCVR, LCVR, and/or CDR amino acid sequences disclosed herein having one or more conservative substitutions. For example, the present invention includes anti-ACVR1 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “human antibody”, or “fully human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, or “fully human antibody”, as used herein, is not intended to include mAbs in which CDR sequences derived from the germline of another mammalian species (e.g., mouse), have been grafted onto human FR sequences. The term includes antibodies that are recombinantly produced in a non-human mammal, or in cells of a non-human mammal. The term is not intended to include antibodies isolated from or generated in a human subject.

The term “recombinant”, as used herein, refers to antibodies or antigen-binding fragments thereof of the invention created, expressed, isolated or obtained by technologies or methods known in the art as recombinant DNA technology which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

The term “specifically binds,” or “binds specifically to”, or the like, means that an antibody or antigen-binding fragment thereof forms a complex with an antigen that is relatively stable under physiologic conditions. Specific binding can be characterized by an equilibrium dissociation constant of at least about 1×10⁻⁸ M or less (e.g., a smaller K_{D} denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to ACVR1. Moreover, multi-specific antibodies that bind to one domain in ACVR1 and one or more additional antigens or a bi-specific that binds to two different regions of ACVR1 are nonetheless considered antibodies that “specifically bind”, as used herein.

The term “high affinity” antibody refers to those mAbs having a binding affinity to ACVR1, expressed as K_{D}, of at least 10⁻⁸ M; preferably 10⁻⁹ M; more preferably 10⁻¹⁰ M, even more preferably 10⁻¹¹ M, as measured by surface plasmon resonance, e.g., BIACORE™ or solution-affinity ELISA.

By the term “slow off rate”, “Koff” or “kd” is meant an antibody that dissociates from ACVR1, with a rate constant of 1×10⁻³ s⁻¹ or less, preferably 1×10⁻⁴ s⁻¹ or less, as determined by surface plasmon resonance, e.g., BIACORE™.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to bind to ACVR1 protein.

In specific embodiments, antibody or antibody fragments of the invention may be conjugated to a moiety such a ligand or a therapeutic moiety (“immunoconjugate”), a second anti-ACVR1 antibody, or any other therapeutic moiety useful for treating a ACVR1-associated disease or disorder.

An “isolated antibody”, as used herein, is intended to refer to an antibody that is substantially free of other antibodies (Abs) having different antigenic specificities (e.g., an isolated antibody that specifically binds ACVR1, or a fragment thereof, is substantially free of Abs that specifically bind antigens other than ACVR1.

An “deactivating antibody” or an “antagonist antibody”, as used herein (or an “antibody that decreases or blocks ACVR1 activity” or “an antibody that destabilizes the activated conformation”), is intended to refer to an antibody whose binding to ACVR1 results in deactivation of at least one biological activity of ACVR1. For example, an antibody of the invention may decrease anemia upon administration to a subject in need thereof.

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “K_{D}”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “cross-competes”, as used herein, means an antibody or antigen-binding fragment thereof binds to an antigen and inhibits or blocks the binding of another antibody or antigen-binding fragment thereof. The term also includes competition between two antibodies in both orientations, i.e., a first antibody that binds and blocks binding of second antibody and vice-versa. In certain embodiments, the first antibody and second antibody may bind to the same epitope. Alternatively, the first and second antibodies may bind to different, but overlapping epitopes such that binding of one inhibits or blocks the binding of the second antibody, e.g., via steric hindrance. Cross-competition between antibodies may be measured by methods known in the art, for example, by a real-time, label-free bio-layer interferometry assay. Cross-competition between two antibodies may be expressed as the binding of the second antibody that is less than the background signal due to self-self binding (wherein first and second antibodies is the same antibody). Cross-competition between 2 antibodies may be expressed, for example, as % binding of the second antibody that is less than the baseline self-self background binding (wherein first and second antibodies is the same antibody).

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains:

cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

As used herein, the term “subject” refers to an animal, preferably a mammal, more preferably a human, in need of amelioration, prevention and/or treatment of a ACVR1-associated disease or disorder such as anemia or ectopic ossification. The term includes human subjects who have or are at risk of having such a disease or disorder.

As used herein, the terms “treat”, “treating”, or “treatment” refer to the reduction or amelioration of the severity of at least one symptom or indication of a ACVR1-associated disease or disorder due to the administration of a therapeutic agent such as an antibody of the present invention to a subject in need thereof. The terms include inhibition of progression of disease or of worsening of a symptom/indication. The terms also include positive prognosis of disease, i.e., the subject may be free of disease or may have reduced disease upon administration of a therapeutic agent such as an antibody of the present invention. The therapeutic agent may be administered at a therapeutic dose to the subject.

The terms “prevent”, “preventing” or “prevention” refer to inhibition of manifestation of a ACVR1-associated disease or disorder or any symptoms or indications of such a disease or disorder upon administration of an antibody of the present invention.

### Antigen-Binding Fragments of Antibodies

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to specifically bind to an ACVR1 protein, a fragment thereof, and/or mutant thereof. An antibody fragment may include a Fab fragment, a F(ab′)₂ fragment, a Fv fragment, a dAb fragment, a fragment containing a CDR, or an isolated CDR. In certain embodiments, the term “antigen-binding fragment” refers to a polypeptide fragment of a multi-specific antigen-binding molecule. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; (Vi) V_{H}-C_{H}2-C_{H}3; (Vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present invention using routine techniques available in the art.

### Preparation of Human Antibodies

Methods for generating human antibodies in transgenic mice are known in the art. Any such known methods can be used in the context of the present invention to make human antibodies that specifically bind to ACVR1.

An immunogen comprising any one of the following can be used to generate antibodies to ACVR1 protein. In certain embodiments, the antibodies of the invention are obtained from mice immunized with a full length, native ACVR1 protein (See, for example, UniProtKB/Swiss-Prot accession number Q04771) or with DNA encoding the protein or fragment thereof. Alternatively, the protein or a fragment thereof may be produced using standard biochemical techniques and modified and used as immunogen.

In some embodiments, the immunogen may be a recombinant ACVR1 protein or fragment thereof expressed in E. coli or in any other eukaryotic or mammalian cells such as Chinese hamster ovary (CHO) cells (for example, SEQ ID NOs: 338-340).

Using VELOCIMMUNE® technology (see, for example, U.S. Pat. No. 6,596,541, Regeneron Pharmaceuticals, VELOCIMMUNE®) or any other known method for generating monoclonal antibodies, high affinity chimeric antibodies to ACVR1 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. As in the experimental section below, the antibodies are characterized and selected for desirable characteristics, including affinity, selectivity, epitope, etc. The mouse constant regions are replaced with a desired human constant region to generate the fully human antibody of the invention, for example wild type or modified IgG1 or IgG4. While the constant region selected may vary according to specific use, high affinity antigen-binding and target specificity characteristics reside in the variable region.

### Bioequivalents

The anti-ACVR1 antibodies and antibody fragments of the present invention encompass proteins having amino acid sequences that vary from those of the described antibodies, but that retain the ability to bind ACVR1 protein. Such variant antibodies and antibody fragments comprise one or more additions, deletions, or substitutions of amino acids when compared to parent sequence, but exhibit biological activity that is essentially equivalent to that of the described antibodies. Likewise, the antibody-encoding DNA sequences of the present invention encompass sequences that comprise one or more additions, deletions, or substitutions of nucleotides when compared to the disclosed sequence, but that encode an antibody or antibody fragment that is essentially bioequivalent to an antibody or antibody fragment of the invention.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, or potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

### Anti-ACVR1 Antibodies Comprising Fc Variants

According to certain embodiments of the present invention, anti-ACVR1 antibodies are provided comprising an Fc domain comprising one or more mutations which enhance or diminish antibody binding to the FcRn receptor, e.g., at acidic pH as compared to neutral pH. For example, the present invention includes anti-ACVR1 antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present invention includes anti-ACVR1 antibodies comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 257I and 311I (e.g., P257I and Q311I); 257I and 434H (e.g., P257I and N434H); 376V and 434H (e.g., D376V and N434H); 307A, 380A and 434A (e.g., T307A, E380A and N434A); and 433K and 434F (e.g., H433K and N434F). All possible combinations of the foregoing Fc domain mutations and other mutations within the antibody variable domains disclosed herein, are contemplated within the scope of the present invention.

The present invention also includes anti-ACVR1 antibodies comprising a chimeric heavy chain constant (C_{H}) region, wherein the chimeric C_{H} region comprises segments derived from the C_{H} regions of more than one immunoglobulin isotype. For example, the antibodies of the invention may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies of the invention comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric C_{H} region as described herein may, in certain embodiments, exhibit modified Fc effector functions without adversely affecting the therapeutic or pharmacokinetic properties of the antibody. (See, e.g., U.S. Patent Application Publication 2014/0243504, the disclosure of which is hereby incorporated by reference in its entirety).

### Biological Characteristics of the Antibodies

In general, the antibodies of the present invention function by binding to ACVR1 protein and decreasing its activity. For example, the present invention includes antibodies and antigen-binding fragments of antibodies that bind human ACVR1 protein (e.g., at 25° C. or at 37° C.) with a K_{D} of less than 500 nM as measured by surface plasmon resonance, e.g., using the assay format as defined in Example 3 herein.

In certain embodiments, the antibodies or antigen-binding fragments thereof bind ACVR1 with a Ko of less than about 500 nM, less than about 400 nM, less than about 300 nM, less than about 200 nM, less than about 100 nM, less than about 50 nM, less than about 25 nM, less than about 10 nM, less than about 5 nM, less than about 3 nM, less than about 2 nM, less than about 1 nM as measured by surface plasmon resonance, e.g., using the assay format as defined in Example 3 herein, or a substantially similar assay. In certain embodiments, the present invention provides an isolated anti-ACVR1 antibody or antigen-binding fragment thereof that is a fully human monoclonal antibody.

In certain embodiments, the antibodies or antigen-binding fragments thereof bind to human ACVR1 extracellular domain fused to an Fc (e.g., SEQ ID NO: 339) at 25° C. with a dissociation constant (K_{D}) of less than 60 nM, less than 12 nM, less than less than 2 nM, less than 1 nM, or less than 0.5 nM as measured in a surface plasmon resonance assay, e.g., using the assay format as defined in Example 3 herein, or a substantially similar assay.

In certain embodiments, the antibodies or antigen-binding fragments thereof bind to human ACVR1 extracellular domain fused to mFc (SEQ ID NO: 339) at 37° C. with a dissociation constant (K_{D}) of less than 150 nM, less than 15 nM, less than less than 5 nM, less than 1.5 nM, or less than 1 nM as measured in a surface plasmon resonance assay, e.g., using the assay format as defined in Example 3 herein, or a substantially similar assay.

In certain embodiments, the antibodies or antigen-binding fragments thereof bind to human ACVR1 extracellular domain fused to myc-myc-hexahistag (e.g., SEQ ID NO: 338) at 25° C. with a K_{D} of less than 300 nM, less than 150 nM, less than 25 nM, less than 10 nM, less than 5 nM, less than 3 nM or less than 2 nM as measured in a surface plasmon resonance assay, e.g., using the assay format as defined in Example 3 herein, or a substantially similar assay.

In certain embodiments, the antibodies or antigen-binding fragments thereof bind to human ACVR1 extracellular domain fused to myc-myc-hexahistag (e.g., SEQ ID NO: 338) at 37° C. with a K_{D} of less than 500 nM, less than 50 nM, less than 25 nM, less than 10 nM, as measured in a surface plasmon resonance assay, e.g., using the assay format as defined in Example 3 herein, or a substantially similar assay.

In certain embodiments, the antibodies or antigen-binding fragments thereof do not bind mouse ACVR1, e.g., using the assay format as defined in Example 3 herein, or a substantially similar assay.

In certain embodiments, the antibodies or antigen-binding fragments thereof bind to mouse ACVR1 extracellular domain fused to myc-myc-hexahistag (e.g., SEQ ID NO: 340) at 25° C. with a K_{D} of greater than 500 nM, as measured in a surface plasmon resonance assay, e.g., using the assay format as defined in Example 3 herein, or a substantially similar assay.

In certain embodiments, the antibodies or antigen-binding fragments thereof bind to mouse ACVR1 extracellular domain fused to myc-myc-hexahistag (e.g., SEQ ID NO: 340) at 37° C. with a K_{D} of greater than 500 nM, as measured in a surface plasmon resonance assay, e.g., using the assay format as defined in Example 3 herein, or a substantially similar assay. The present invention also includes antibodies or antigen-binding fragments thereof bind to cells expressing human ACVR1 protein or human ACVR (R206H) protein, e.g., using the assay format as defined in Example 5 herein, or a substantially similar assay.

In certain embodiments, the antibodies or antigen-binding fragments thereof inhibit activation of cells expressing human ACVR1(R206H) by human Activin A with a IC₅₀ of less than 25 nM, as measured in a cell-based bioassay, e.g., using the assay format as defined in Example 6 herein, or a substantially similar assay.

In certain embodiments, the antibodies or antigen-binding fragments thereof inhibit activation of cells expressing human ACVR1(R206H) by human BMP7 with a IC₅₀ of less than 20 nM, less than 5 nM, less than 3 nM, or less than 1 nM, or less than as measured in a cell-based bioassay, e.g., using the assay format as defined in Example 6 herein, or a substantially similar assay.

The invention also includes antibodies or antigen-binding fragments thereof that significantly decrease serum hepcidin when administered to mice expressing human ACVR1 in place of mouse allele, e.g., using the assay format as defined in Example 7 herein, or a substantially similar assay.

In certain embodiments, the antibodies or antigen-binding fragments thereof that significantly increase serum iron levels when administered to mice expressing human ACVR1 in place of mouse allele, e.g., using the assay format as defined in Example 7 herein, or a substantially similar assay.

In certain embodiments, the antibodies or antigen-binding fragments thereof inhibit wild-type ACVR1 signaling when administered to mice expressing human ACVR1 in place of mouse allele, e.g., using the assay format as defined in Example 7 herein, or a substantially similar assay.

In certain embodiments, the anti-ACVR antibodies or antigen-binding fragments thereof according to the invention significantly attenuate heterotopic ossification (HO) in a post-traumatic HO model in wild type mice, e.g., as described in Example 8 herein, or a substantially similar model.

In certain embodiments, the antibodies or antigen-binding fragments thereof specifically bind human ACVR1, a fragment thereof, or a mutant thereof, and comprise a HCVR comprising an amino acid sequence selected from the group consisting of HCVR sequence listed in Table 1 and a LCVR comprising an amino acid sequence selected from the group consisting of LCVR sequences listed in Table 1.

In one embodiment, the present invention provides an isolated recombinant antibody or antigen-binding fragment thereof that binds specifically to ACVR1 protein and inhibit ACVR1-mediated bone morphogenetic protein (BMP) signal transduction, wherein the antibody or fragment thereof exhibits one or more of the following characteristics: (a) is a fully human monoclonal antibody; (b) binds to human ACVR1 extracellular domain fused to an Fc (e.g., SEQ ID NO: 339) at 25° C.with a dissociation constant (K_{D}) of less than 60 nM, less than 12 nM, less than less than 2 nM, less than 1 nM, or less than 0.5 nM as measured in a surface plasmon resonance assay; (c) binds to human ACVR1 extracellular domain fused to mFc (SEQ ID NO: 339) at 37° C. with a dissociation constant (K_{D}) of less than 150 nM, less than 15 nM, less than less than 5 nM, less than 1.5 nM, or less than 1 nM as measured in a surface plasmon resonance assay; (d) binds to human ACVR1 extracellular domain fused to myc-myc-hexahistag (e.g., SEQ ID NO: 338) at 25° C. with a K_{D} of less than 300 nM, less than 150 nM, less than 25 nM, less than 10 nM, less than 5 nM, less than 3 nM or less than 2 nM as measured in a surface plasmon resonance assay; (e) binds to human ACVR1 extracellular domain fused to myc-myc-hexahistag (e.g., SEQ ID NO: 338) at 37° C. with a K_{D} of less than 500 nM, less than 50 nM, less than 25 nM, less than 10 nM, as measured in a surface plasmon resonance assay; (f) does not bind mouse ACVR1 or binds to mouse ACVR1 extracellular domain fused to myc-myc-hexahistag (e.g., SEQ ID NO: 340) at 25° C. with a K_{D} of greater than 500 nM, as measured in a surface plasmon resonance assay; (g) does not bind mouse ACVR1 or binds to mouse ACVR1 extracellular domain fused to myc-myc-hexahistag (e.g., SEQ ID NO: 340) at 37° C. with a K_{D} of greater than 500 nM, as measured in a surface plasmon resonance assay; (k) binds to cells expressing human ACVR1 protein or human ACVR (R206H) protein; (I) inhibits activation of cells expressing human ACVR1(R206H) by human Activin A with a IC₅₀ of less than 25 nM, as measured in a cell-based bioassay; (m) inhibits activation of cells expressing human ACVR1(R206H) by human BMP7 with a IC₅₀ of less than 20 nM, less than 5 nM, less than 3 nM, or less than 1 nM, or less than as measured in a cell-based bioassay; (m) significantly decreases serum hepcidin when administered to mice expressing human ACVR1 in place of mouse allele; (n) significantly increases serum iron levels when administered to mice expressing human ACVR1 in place of mouse allele; and/or (o) inhibits wild-type ACVR1 signaling when administered to mice expressing human ACVR1 in place of mouse allele; and (p) comprises a HCVR comprising an amino acid sequence selected from the group consisting of HCVR sequence listed in Table 1 and a LCVR comprising an amino acid sequence selected from the group consisting of LCVR sequences listed in Table 1.

The antibodies of the present invention may possess one or more of the aforementioned biological characteristics, or any combinations thereof. Other biological characteristics of the antibodies of the present invention will be evident to a person of ordinary skill in the art from a review of the present disclosure including the working Examples herein.

### Epitope Mapping and Related Technologies

The present invention includes anti-ACVR1 antibodies which interact with one or more amino acids found within one or more regions of the ACVR1 protein molecule. The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within any of the aforementioned domains of the ACVR1 protein molecule (e.g. a linear epitope in a domain). Alternatively, the epitope may consist of a plurality of non-contiguous amino acids (or amino acid sequences) located within either or both of the aforementioned domains of the protein molecule (e.g. a conformational epitope).

Various techniques known to persons of ordinary skill in the art can be used to determine whether an antibody “interacts with one or more amino acids” within a polypeptide or protein.

Exemplary techniques include, for example, routine cross-blocking assays, such as that described in Antibodies, Harlow and Lane (Cold Spring Harbor Press, Cold Spring Harbor, NY). Other methods include alanine scanning mutational analysis, peptide blot analysis (Reineke (2004) Methods Mol. Biol. 248: 443-63), peptide cleavage analysis crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the invention into groups of antibodies binding different epitopes.

In certain embodiments, the present invention includes anti-ACVR1 antibodies and antigen-binding fragments thereof that interact with one or more epitopes found within the extracellular domain of ACVR1. The epitope(s) may consist of one or more contiguous sequences of 3 or more (e.g., 3, 4, 5, 6, 7, 8, 9, 10, 11, 12, 13, 14, 15, 16, 17, 18, 19, 20 or more) amino acids located within the extracellular domain of ACVR1. Alternatively, the epitope may consist of a plurality of non-contiguous amino acids (or amino acid sequences) located within ACVR1 protein.

The present invention includes anti-ACVR1 antibodies that bind to the same epitope, or a portion of the epitope, as any of the specific exemplary antibodies listed in Table 1. Likewise, the present invention also includes anti-ACVR1 antibodies that compete for binding to ACVR1 protein or a fragment thereof with any of the specific exemplary antibodies listed in Table 1. For example, the present invention includes anti-ACVR1 antibodies that cross-compete for binding toACV protein with one or more antibodies listed in Table 1.

One can easily determine whether an antibody binds to the same epitope as, or competes for binding with, a reference anti-ACVR1 antibody by using routine methods known in the art. For example, to determine if a test antibody binds to the same epitope as a reference anti-ACVR1 antibody of the invention, the reference antibody is allowed to bind to a ACVR1 protein or peptide under saturating conditions. Next, the ability of a test antibody to bind to the ACVR1 protein molecule is assessed. If the test antibody is able to bind to ACVR1 following saturation binding with the reference anti-ACVR1 antibody, it can be concluded that the test antibody binds to a different epitope than the reference anti-ACVR1 antibody. On the other hand, if the test antibody is not able to bind to the ACVR1 protein following saturation binding with the reference anti-ACVR1 antibody, then the test antibody may bind to the same epitope as the epitope bound by the reference anti-ACVR1 antibody of the invention.

To determine if an antibody competes for binding with a reference anti-ACVR1 antibody, the above-described binding methodology is performed in two orientations: In a first orientation, the reference antibody is allowed to bind to a ACVR1 protein under saturating conditions followed by assessment of binding of the test antibody to the ACVR1 molecule. In a second orientation, the test antibody is allowed to bind to a ACVR1 molecule under saturating conditions followed by assessment of binding of the reference antibody to the ACVR1 molecule. If, in both orientations, only the first (saturating) antibody is capable of binding to the ACVR1 molecule, then it is concluded that the test antibody and the reference antibody compete for binding to ACVR1. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20-or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

### Immunoconjugates

The invention encompasses a human anti-ACVR1 monoclonal antibody conjugated to a therapeutic moiety (“immunoconjugate”), to treat an ACVR1-associated disease or disorder (e.g., anemia or ectopic ossification). As used herein, the term “immunoconjugate” refers to an antibody which is chemically or biologically linked to a radioactive agent, a cytokine, an interferon, a target or reporter moiety, an enzyme, a peptide or protein or a therapeutic agent. The antibody may be linked to the radioactive agent, cytokine, interferon, target or reporter moiety, enzyme, peptide or therapeutic agent at any location along the molecule so long as it is able to bind its target. Examples of immunoconjugates include antibody drug conjugates and antibody-toxin fusion proteins. In one embodiment, the agent may be a second different antibody to ACVR 1 protein. The type of therapeutic moiety that may be conjugated to the anti-ACVR1 antibody and will take into account the condition to be treated and the desired therapeutic effect to be achieved. Examples of suitable agents for forming immunoconjugates are known in the art; see for example, WO 05/103081.

### Multi-Specific Antibodies

The antibodies of the present invention may be mono-specific, bi-specific, or multi-specific. Multi-specific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244.

Any of the multi-specific antigen-binding molecules of the invention, or variants thereof, may be constructed using standard molecular biological techniques (e.g., recombinant DNA and protein expression technology), as will be known to a person of ordinary skill in the art.

In some embodiments, ACVR1-specific antibodies are generated in a bi-specific format (a “bi-specific”) in which variable regions binding to distinct domains of ACVR1 protein are linked together to confer dual-domain specificity within a single binding molecule. Appropriately designed bi-specifics may enhance overall ACVR1-protein inhibitory efficacy through increasing both specificity and binding avidity. Variable regions with specificity for individual domains, (e.g., segments of the N-terminal domain), or that can bind to different regions within one domain, are paired on a structural scaffold that allows each region to bind simultaneously to the separate epitopes, or to different regions within one domain. In one example for a bi-specific, heavy chain variable regions (V_{H}) from a binder with specificity for one domain are recombined with light chain variable regions (V_{L}) from a series of binders with specificity for a second domain to identify non-cognate V_{L} partners that can be paired with an original V_{H} without disrupting the original specificity for that V_{H}. In this way, a single V_{L} segment (e.g., V_{L}1) can be combined with two different V_{H} domains (e.g., V_{H}1 and V_{H}2) to generate a bi-specific comprised of two binding “arms” (V_{H}1-V_{L}1 and V_{H}2-V_{L}1). Use of a single V_{L} segment reduces the complexity of the system and thereby simplifies and increases efficiency in cloning, expression, and purification processes used to generate the bi-specific (See, for example, US2011/0195454 and US2010/0331527).

Alternatively, antibodies that bind more than one domains and a second target, such as, but not limited to, for example, a second different anti-ACVR1 antibody, may be prepared in a bi-specific format using techniques described herein, or other techniques known to those skilled in the art. Antibody variable regions binding to distinct regions may be linked together with variable regions that bind to relevant sites on, for example, the extracellular domain of ACVR1, to confer dual-antigen specificity within a single binding molecule. Appropriately designed bi-specifics of this nature serve a dual function. Variable regions with specificity for the extracellular domain are combined with a variable region with specificity for outside the extracellular domain and are paired on a structural scaffold that allows each variable region to bind to the separate antigens.

An exemplary bi-specific antibody format that can be used in the context of the present invention involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bi-specific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V821 (by IMGT; D356E, L358M, N384S, K392N, V397M, and V422I by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V422I by EU) in the case of IgG4 antibodies. Variations on the bi-specific antibody format described above are contemplated within the scope of the present invention.

Other exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

### Therapeutic Administration and Formulations

The invention provides therapeutic compositions comprising the anti-ACVR1 antibodies or antigen-binding fragments thereof of the present invention. Therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody may vary depending upon the age and the size of a subject to be administered, target disease, conditions, route of administration, and the like. When an antibody of the present invention is used for treating a disease or disorder in an adult patient, or for preventing such a disease, it is advantageous to administer the antibody of the present invention normally at a single dose of about 0.1 to about 100 mg/kg body weight. Depending on the severity of the condition, the frequency and the duration of the treatment can be adjusted. In certain embodiments, the antibody or antigen-binding fragment thereof of the invention can be administered as an initial dose of at least about 0.1 mg to about 800 mg, about 1 to about 600 mg, about 5 to about 500 mg, or about 10 to about 400 mg. In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural, intracerebroventricular, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can be also delivered in a vesicle, in particular a liposome (see, for example, Langer (1990) Science 249:1527-1533).

The use of nanoparticles to deliver the antibodies of the present invention is also contemplated herein. Antibody-conjugated nanoparticles may be used both for therapeutic and diagnostic applications. Antibody-conjugated nanoparticles and methods of preparation and use are described in detail by Arruebo, M., et al. 2009 (“Antibody-conjugated nanoparticles for biomedical applications” in J. Nanomat. Volume 2009, Article ID 439389, 24 pages, doi: 10.1155/2009/439389), incorporated herein by reference. Nanoparticles may be developed and conjugated to antibodies contained in pharmaceutical compositions to target cells. Nanoparticles for drug delivery have also been described in, for example, U.S. Pat. No. 8,257,740, or U.S. Pat. No. 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracranial, intraperitoneal and intramuscular injections, drip infusions, etc. These injectable preparations may be prepared by methods publicly known.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In treatment of DIPG, it may be necessary to overcome the blood-brain barrier. In certain embodiments, the blood-brain barrier is overcome by using one or more approaches disclosed in the art, e.g., in Parodi et al 2019, Pharmaceutics 11:245.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 500 mg per dosage form in a unit dose; especially in the form of injection, it is preferred that the antibody is contained in about 5 to about 300 mg and in about 10 to about 300 mg for the other dosage forms.

### Therapeutic Uses of the Antibodies

The antibodies of the present invention are useful for the treatment, and/or prevention of a disease or disorder or condition associated with ACVR1 and/or for ameliorating at least one symptom associated with such disease, disorder or condition. In certain embodiments, an antibody or antigen-binding fragment thereof of the invention may be administered at a therapeutic dose to a patient with a disease or disorder or condition associated with ACVR1 or a mutant ACVR protein.

In certain embodiments, the antibodies of the present invention are useful for treating or preventing at least one symptom or indication of an ACVR1-associated or ACVR1 mutant protein-associated disease or disorder selected from the group consisting of heterotopic ossification, ectopic ossification, bone dysplasia, anemia, and diffuse intrinsic pontine glioma.

It is also contemplated herein to use one or more antibodies of the present invention prophylactically to subjects at risk for suffering from a ACVR1-associated disease or disorder.

In one embodiment of the invention, the present antibodies are used for the preparation of a pharmaceutical composition or medicament for treating patients suffering from a disease, disorder or condition disclosed herein. In another embodiment of the invention, the present antibodies are used as adjunct therapy with any other agent or any other therapy known to those skilled in the art useful for treating or ameliorating a disease, disorder or condition disclosed herein.

### Combination Therapies

Combination therapies may include an antibody of the invention and any additional therapeutic agent that may be advantageously combined with an antibody of the invention, or with a biologically active fragment of an antibody of the invention. The antibodies of the present invention may be combined synergistically with one or more drugs or therapy used to treat an ACVR1-associated or ACVR1 mutant protein-associated disease or disorder. In some embodiments, the antibodies of the invention may be combined with a second therapeutic agent to ameliorate one or more symptoms of said disease or condition.

Depending upon the disease, disorder or condition, the antibodies of the present invention may be used in combination with one or more additional therapeutic agents.

Examples of the additional therapeutic drug for ectopic ossification that can be administered in combination with the anti-ACVR 1 antibody can include, but are not limited to, anti-Activin A inhibitor or antigen binding fragment thereof, and an anti-ACVR2 antibody or antigen-binding fragment thereof, anti-inflammatory drugs, steroids, bisphosphonates, muscle relaxants, and retinoic acid receptor (RAR) gamma agonists.

Activins belong to the transforming growth factor-beta (TGF-β) superfamily and exert a broad range of biological effects on cell proliferation, differentiation, metabolism, homeostasis, and apoptosis, as well as immune response and tissue repair. Activin A is a disulfide-linked homodimer (two beta-A chains) that binds to and activates heteromeric complexes of a type I (Act RI-A and Act RI-B) and a type II (Act RII-A and Act RII-B) serine-threonine kinase receptor. Activin A may act as a ligand to ACVR1 proteins or ACVR1 mutant proteins.

Examples of the anti-inflammatory drug can include aspirin, diclofenac, indomethacin, ibuprofen, ketoprofen, naproxen, piroxicam, rofecoxib, celecoxib, azathioprine, penicillamine, methotrexate, sulfasalazine, leflunomide, infliximab, and etanercept. Examples of the steroid can include prednisolone, beclomethasone, betamethasone, fluticasone, dexamethasone, and hydrocortisone. Examples of the bisphosphonate can include alendronate, cimadronate, clodronate, etidronate, ibandronate, incadronate, minodronate, neridronate, olpadronate, pamidronate, piridronate, risedronate, tiludronate, and zoledronate. Examples of the muscle relaxant can include cyclobenzaprine, metaxalone, and baclofen. Examples of the retinoic acid receptor gamma agonist can include palovarotene. Examples of the additional therapeutic drug for anemia may include recombinant erythropoietin (EPO) and iron supplements. Examples of additional therapeutic treatments for diffuse intrinsic pontine glioma may include radiation therapy, or experimental chemotherapy.

As used herein, the term “in combination with” means that additional therapeutically active component(s) may be administered prior to, concurrent with, or after the administration of the anti-ACVR1 antibody of the present invention. The term “in combination with” also includes sequential or concomitant administration of an anti-ACVR1 antibody and a second therapeutic agent.

The additional therapeutically active component(s) may be administered to a subject prior to administration of an anti-ACVR1 antibody of the present invention. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, or less than 30 minutes before administration of the second component. In other embodiments, the additional therapeutically active component(s) may be administered to a subject after administration of an anti-ACVR1 antibody of the present invention. For example, a first component may be deemed to be administered “after” a second component if the first component is administered 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after or more after administration of the second component. In yet other embodiments, the additional therapeutically active component(s) may be administered to a subject concurrent with administration of an anti-ACVR1 antibody of the present invention. “Concurrent” administration, for purposes of the present invention, includes, e.g., administration of an anti-ACVR1 antibody and an additional therapeutically active component to a subject in a single dosage form, or in separate dosage forms administered to the subject within about 30 minutes or less of each other. If administered in separate dosage forms, each dosage form may be administered via the same route (e.g., both the anti-ACVR1 antibody and the additional therapeutically active component may be administered intravenously, etc.); alternatively, each dosage form may be administered via a different route (e.g., the anti-ACVR1 antibody may be administered intravenously, and the additional therapeutically active component may be administered orally). In any event, administering the components in a single dosage from, in separate dosage forms by the same route, or in separate dosage forms by different routes are all considered “concurrent administration,” for purposes of the present disclosure. For purposes of the present disclosure, administration of an anti-ACVR1 antibody “prior to”, “concurrent with,” or “after” (as those terms are defined herein above) administration of an additional therapeutically active component is considered administration of an anti-ACVR1 antibody “in combination with” an additional therapeutically active component.

The present invention includes pharmaceutical compositions in which an anti-ACVR1 antibody of the present invention is co-formulated with one or more of the additional therapeutically active component(s) as described elsewhere herein.

### Diagnostic Uses of the Antibodies

The antibodies of the present invention may be used to detect and/or measure ACVR1 protein in a sample, e.g., for diagnostic purposes. Some embodiments contemplate the use of one or more antibodies of the present invention in assays to detect a ACVR 1-associated-or ACVR mutant-protein-associated-disease or disorder. Exemplary diagnostic assays for ACVR1 may comprise, e.g., contacting a sample, obtained from a patient, with an anti-ACVR1 antibody of the invention, wherein the anti-ACVR1 antibody is labeled with a detectable label or reporter molecule or used as a capture ligand to selectively isolate ACVR1 from patient samples. Alternatively, an unlabeled anti-ACVR1 antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure ACVR1 in a sample include enzyme-linked immunosorbent assay (ELISA), radioimmunoassay (RIA), and fluorescence-activated cell sorting (FACS).

Samples that can be used in ACVR1 diagnostic assays according to the present invention include any tissue or fluid sample obtainable from a patient, which contains detectable quantities of either ACVR1 protein, or fragments thereof, under normal or pathological conditions. Generally, levels of ACVR1 protein in a particular sample obtained from a healthy patient (e.g., a patient not afflicted with a disease associated with ACVR1) will be measured to initially establish a baseline, or standard, level of ACVR1. This baseline level of ACVR1 can then be compared against the levels of ACVR1 measured in samples obtained from individuals suspected of having a ACVR1-associated condition, or symptoms associated with such condition.

The antibodies specific for ACVR1 protein may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Generation of Human Antibodies to Activin A Receptor Receptor 1 (ACVR1)

Human antibodies to ACVR1 protein were generated in a VELOCIMMUNE® mouse comprising DNA encoding human Immunoglobulin heavy and kappa light chain variable regions. The mice were immunized with an immunogen comprising extracellular domain of human ACVR1 protein (e.g., SEQ ID NO: 339).

The antibody immune response was monitored by a ACVR1-specific immunoassay. When a desired immune response was achieved splenocytes were harvested and fused with mouse myeloma cells to preserve their viability and form hybridoma cell lines. The hybridoma cell lines were screened and selected to identify cell lines that produce ACVR1-specific antibodies. The cell lines were used to obtain several anti-ACVR1 chimeric antibodies (i.e., antibodies possessing human variable domains and mouse constant domains).

Anti-ACVR1 antibodies were also isolated directly from antigen-positive mouse B cells without fusion to myeloma cells, as described in U.S. Pat. No. 7,582,298, herein specifically incorporated by reference in its entirety. Using this method, several fully human anti-ACVR1 antibodies (i.e., antibodies possessing human variable domains and human constant domains) were obtained.

Exemplary antibodies generated as disclosed above were designated as mAb27396, mAb27241, mAb27242, mAb27243, mAb27245, mAb27247, mAb27404, mAb27405, mAb27400, mAb22124, mAb22125, mAb22168, mAb29226, mAb29226, mAb29237, mAb29256, mAb29257, mAb29261, mAb29266, mAb22115.

The biological properties of the exemplary antibodies generated in accordance with the methods of this Example are described in detail in the Examples set forth below.

### Example 2: Heavy and Light Chain Variable Region Amino Acid and Nucleotide Sequences

Table 1 sets forth the amino acid sequence identifiers of the heavy and light chain variable regions and CDRs of selected anti-ACVR1 antibodies of the invention.

TABLE 1
Amino Acid Sequence Identifiers

| Antibody | SEQ ID NO: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb27396 | 2 | 4 | 6 | 8 | 10 | 12 | 14 | 16 |
| | | | | | | | Gly Ala | |
| | | | | | | | Ser | |
| mAb27241 | 22 | 24 | 26 | 28 | 30 | 32 | 34 | 36 |
| | | | | | | | Asp Val | |
| | | | | | | | Ser | |
| mAb27242 | 42 | 44 | 46 | 28 | 48 | 50 | 52 | 36 |
| | | | | | | | Asp Val | |
| | | | | | | | Phe | |
| mAb27243 | 58 | 24 | 46 | 60 | 62 | 50 | 64 | 66 |
| | | | | | | | Asp Ala | |
| | | | | | | | Ser | |
| mAb27245 | 22 | 24 | 26 | 28 | 72 | 50 | 64 | 66 |
| | | | | | | | Asp Ala | |
| | | | | | | | Ser | |
| mAb27247 | 76 | 78 | 80 | 82 | 84 | 86 | 88 | 90 |
| | | | | | | | Gln Val | |
| | | | | | | | Ser | |
| mAb27404 | 96 | 98 | 100 | 102 | 104 | 106 | 108 | 110 |
| | | | | | | | Thr Val | |
| | | | | | | | Ser | |
| mAb27405 | 116 | 98 | 100 | 102 | 119 | 106 | 122 | 110 |
| | | | | | | | Thr Ile | |
| | | | | | | | Ser | |
| mAb27400 | 128 | 130 | 132 | 134 | 136 | 138 | 140 | 142 |
| | | | | | | | Lys Ala | |
| | | | | | | | Ser | |
| mAb22124 | 148 | 150 | 152 | 154 | 156 | 158 | 14 | 160 |
| mAb22125 | 166 | 168 | 170 | 172 | 174 | 176 | 178 | 180 |
| | | | | | | | Ala Ala | |
| | | | | | | | Ser | |
| mAb22168 | 186 | 188 | 152 | 191 | 193 | 195 | 178 | 197 |
| mAb29226 | 203 | 205 | 207 | 209 | 211 | 213 | 215 | 217 |
| | | | | | | | Trp Ala | |
| | | | | | | | Ser | |
| mAb29233 | 223 | 225 | 227 | 229 | 231 | 233 | 178 | 235 |
| mAb29237 | 241 | 205 | 243 | 209 | 245 | 233 | 247 | 249 |
| | | | | | | | Thr Ala | |
| | | | | | | | Ser | |
| mAb29256 | 255 | 257 | 259 | 261 | 263 | 265 | 14 | 267 |
| mAb29257 | 273 | 257 | 275 | 261 | 277 | 279 | 281 | 283 |
| | | | | | | | Glu Ile | |
| | | | | | | | Ser | |
| mAb29261 | 289 | 257 | 291 | 261 | 293 | 233 | 178 | 235 |
| mAb29266 | 300 | 4 | 303 | 305 | 307 | 309 | 311 | 313 |
| | | | | | | | Val Ala | |
| | | | | | | | Ser | |
| mAb22115 | 319 | 321 | 323 | 325 | 327 | 329 | 331 | 333 |
| | | | | | | | Lys Val | |
| | | | | | | | Ser | |

The corresponding nucleic acid sequence identifiers are set forth in Table 2.

TABLE 2
Nucleic Acid Sequence Identifiers

| Antibody | SEQ ID NO: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb27396 | 1 | 3 | 5 | 7 | 9 | 11 | 13 | 15 |
| mAb27241 | 21 | 23 | 25 | 27 | 29 | 31 | 33 | 35 |
| mAb27242 | 41 | 43 | 45 | 27 | 47 | 49 | 51 | 35 |
| mAb27243 | 57 | 23 | 45 | 59 | 61 | 49 | 63 | 65 |
| mAb27245 | 21 | 23 | 25 | 27 | 71 | 49 | 63 | 65 |
| mAb27247 | 75 | 77 | 79 | 81 | 83 | 85 | 87 | 89 |
| mAb27404 | 95 | 97 | 99 | 101 | 103 | 105 | 107 | 109 |
| mAb27405 | 115 | 117 | 99 | 101 | 118 | 120 | 121 | 109 |
| mAb27400 | 127 | 129 | 131 | 133 | 135 | 137 | 139 | 141 |
| mAb22124 | 147 | 149 | 151 | 153 | 155 | 157 | 13 | 159 |
| mAb22125 | 165 | 167 | 169 | 171 | 173 | 175 | 177 | 179 |
| mAb22168 | 185 | 187 | 189 | 190 | 192 | 194 | 177 | 196 |
| mAb29226 | 202 | 204 | 206 | 208 | 210 | 212 | 214 | 216 |
| mAb29233 | 222 | 224 | 226 | 228 | 230 | 232 | 177 | 234 |
| mAb29237 | 240 | 204 | 242 | 208 | 244 | 232 | 246 | 248 |
| mAb29256 | 254 | 256 | 258 | 260 | 262 | 264 | 13 | 266 |
| mAb29257 | 272 | 256 | 274 | 260 | 276 | 278 | 280 | 282 |
| mAb29261 | 288 | 256 | 290 | 260 | 292 | 232 | 177 | 294 |
| mAb29266 | 299 | 301 | 302 | 304 | 306 | 308 | 310 | 312 |
| mAb22115 | 318 | 320 | 322 | 324 | 326 | 328 | 330 | 332 |

Antibodies referred to herein typically have fully human variable regions, but may have human or mouse constant regions. As will be appreciated by a person of ordinary skill in the art, an antibody having a particular Fc isotype can be converted to an antibody with a different Fc isotype (e.g., an antibody with a mouse IgG1 Fc can be converted to an antibody with a human IgG4, etc.), but in any event, the variable domains (including the CDRs)—which are indicated by the numerical identifiers shown in Tables 1 or 2—will remain the same, and the binding properties to antigen are expected to be identical or substantially similar regardless of the nature of the Fc domain. In certain embodiments, selected antibodies with a mouse IgG1 Fc are converted to antibodies with human IgG4 Fc. In one embodiment, the IgG4 Fc domain comprises 2 or more amino acid changes as disclosed in US20100331527. In one embodiment, the human IgG4 Fc comprises a serine to proline mutation in the hinge region (S108P) to promote dimer stabilization. Unless indicated otherwise, all antibodies used in the following examples comprise a human IgG4 isotype.

Table 3 sets forth the nucleic acid (DNA) and amino acid (PEP) sequence identifiers of the heavy and light chains (HC and LC) of selected anti-ACVR1 antibodies of the invention.

TABLE 3
Sequence Identifiers for Heavy and Light Chains

| Antibody | SEQ ID NO: | | | |
|---|---|---|---|---|
| Designation | HC DNA | HC PEP | LC DNA | LC PEP |
| mAb27396 | 17 | 18 | 19 | 20 |
| mAb27241 | 37 | 38 | 39 | 40 |
| mAb27242 | 53 | 54 | 55 | 56 |
| mAb27243 | 67 | 68 | 69 | 70 |
| mAb27245 | 37 | 38 | 73 | 74 |
| mAb27247 | 91 | 92 | 93 | 94 |
| mAb27404 | 111 | 112 | 113 | 114 |
| mAb27405 | 123 | 124 | 125 | 126 |
| mAb27400 | 143 | 144 | 145 | 146 |
| mAb22124 | 161 | 162 | 163 | 164 |
| mAb22125 | 181 | 182 | 183 | 184 |
| mAb22168 | 198 | 199 | 200 | 201 |
| mAb29226 | 218 | 219 | 220 | 221 |
| mAb29233 | 236 | 237 | 238 | 239 |
| mAb29237 | 250 | 251 | 252 | 253 |
| mAb29256 | 268 | 269 | 270 | 271 |
| mAb29257 | 284 | 285 | 286 | 287 |
| mAb29261 | 295 | 296 | 297 | 298 |
| mAb29266 | 314 | 315 | 316 | 317 |
| mAb22115 | 334 | 335 | 336 | 337 |

### Example 3: Antibody Binding to ACVR1 as Determined by Surface Plasmon Resonance

### Experimental Procedure

Equilibrium dissociation constants (K_{D}) for ACVR1 binding to purified anti-ACVR1 monoclonal antibodies were determined using a real-time surface plasmon resonance biosensor (SPR-Biacore), Biacore 4000. All binding studies were performed in 10 mM HEPES, 150 mM NaCl, 3 mM EDTA, and 0.05% v/v surfactant Tween-20, pH 7.4 (HBS-ET) running buffer at 25° C. and 37° C. The Biacore CM5 sensor surface was first derivatized by amine coupling with a monoclonal mouse anti-human Fc antibody (GE, # BR-1008-39 or REGN2567) to capture anti-ACVR1 monoclonal antibodies. Different concentrations of ACVR1 reagents, human ACVR1 extracellular domain expressed with a C-terminal myc-myc-hexahistidine tag (hACVR1-MMH; SEQ ID NO: 338), mouse ACVR1 extracellular domain expressed with a C-terminal myc-myc-hexahistidine tag (mACVR1-MMH; SEQ ID NO: 340), human ACVR1 extracellular domain expressed with a C-terminal mouse IgG2a Fc tag (hACVR1-mFc; SEQ ID NO: 339), were first prepared in HBS-ET running buffer (900 nM-3.7 nM; serially diluted by 3-fold). ACVR1 reagents were then injected over anti-human Fc captured anti-ACVR1 monoclonal antibody surface for 2.5-3 minutes at a flow rate of 30 μL/minute, while the dissociation of monoclonal antibody bound ACVR1 reagent was monitored for 10-15 minutes in HBS-ET running buffer. Kinetic association rate constant (kₐ) and dissociation rate constant (k_{d}) were determined by fitting the real-time sensorgrams to a 1:1 binding model using Scrubber 2.0c curve-fitting software. Binding dissociation equilibrium constants (K_{D}) and dissociative half-lives (t½) were calculated from the kinetic rate constants as:

$$\\ {K}_{D} ( M ) = \frac{kd}{ka} , and t 1 / 2 ( min ) = \frac{ln ( 2 )}{60 * kd}$$

### Results

Binding kinetics parameters for different ACVR1 reagents to anti-ACVR1 monoclonal antibodies of the invention at 25° C. and 37° C. are shown in Table 4 through Table 9.

TABLE 4
Binding kinetics parameters of hACVR1-MMH binding to anti-ACVR1
monoclonal antibodies at 25° C.

| | | 900 nM or | | | | |
|---|---|---|---|---|---|---|
| | mAb Capture | 100 nM Ag | kₐ | k_{d} | K_{D} | t_{1/2} |
| mAb captured | Level (RU) | Bound (RU) | (1/Ms) | (1/s) | (M) | (min) |
| mAb29257 | 350 + 4.6 | 47 | 6.18E+04 | 5.21E−04 | 8.43E−09 | 22 |
| mAb27396 | 349 + 4.5 | 23 | 2.56E+05 | 7.45E−-02 | 2.91E−07 | 0.16 |
| mAb29226 | 344 + 3.4 | 57 | 1.13E+05 | 9.37E−04 | 8.27E−09 | 12 |
| MAB27405 | 366 + 2.2 | 37 | 6.99E+04 | 1.47E−03 | 2.10E−08 | 8 |
| H4H27247 | 349 + 2.8 | 52 | 1.15E+05 | 1.19E−03 | 1.04E−08 | 10 |
| mAb27404 | 366 + 1.1 | 46 | 9.92E+04 | 4.46E−04 | 4.49E−09 | 26 |
| mAb27242 | 363 + 1.9 | 63 | 8.68E+04 | 1.35E−04 | 1.56E−09 | 86 |
| mAb27243 | 353 + 2.8 | 51 | 5.41E+04 | 1.60E−04 | 2.96E−09 | 72 |
| mAb27245 | 364 + 3 | 35 | 2.58E+04 | 2.07E−04 | 8.01E−09 | 56 |
| mAb27241 | 346 + 2 | 34 | 1.89E+04 | 1.23E−04 | 6.52E−09 | 94 |
| mAb29266 | 351 + 3 | 44 | 6.21E+04 | 6.79E−03 | 1.09E−07 | 1.7 |
| mAb27400 | 352 + 3.4 | 2 | NB* | NB* | NB* | NB* |
| mAb22125 | 687 + 2.1 | 44 | 4.13E+05 | 4.20E−01 | 1.02E−06 | 0.03 |
| mAb22124 | 641 + 1.8 | 21 | 6.61E+05 | 1.30E+00 | 1.97E−06 | 0.01 |
| mAb22168 | 646 + 0.9 | 36 | 7.98E+05 | 8.43E−01 | 1.06E−06 | 0.01 |
| mAb29233 | 370 + 1.1 | 1 | NB* | NB* | NB* | NB* |
| mAb29237 | 305 + 3.2 | 0 | NB* | NB* | NB* | NB* |
| mAb29256 | 332 + 2.3 | −2 | NB* | NB* | NB* | NB* |
| mAb29261 | 332 + 3.6 | 0 | NB* | NB* | NB* | NB* |
| mAb22115 | 630 + 1.5 | 93 | 3.34E+04 | 4.85E−03 | 1.45E−07 | 2.4 |
| Isotype | 637 + 0.5 | 1 | NB* | NB* | NB* | NB* |
| Control mAb | | | | | | |

*NB indicates that no binding was observed under the current experimental conditions.

TABLE 5
Binding kinetics parameters of hACVR1-MMH binding to
anti-ACVR1 monoclonal antibodies at 37° C.

| | | 900 nM or | | | | |
|---|---|---|---|---|---|---|
| | mAb Capture | 100 nM Ag | kₐ | k_{d} | K_{D} | t_{1/2} |
| mAb captured | Level (RU) | Bound (RU) | (1/Ms) | (1/s) | (M) | (min) |
| mAb29257 | 431 + 1.5 | 64 | 1.53E+05 | 3.26E−03 | 2.14E−08 | 3.5 |
| mAb27396 | 451 + 4.6 | 22 | 4.96E+05 | 1.80E−01 | 3.62E−07 | 0.06 |
| mAb29226 | 397 + 1.8 | 68 | 1.82E+05 | 4.56E−03 | 2.50E−08 | 2.5 |
| mAb27405 | 440 + 1.3 | 52 | 1.19E+05 | 5.28E−03 | 4.44E−08 | 2.2 |
| mAb27247 | 404 + 2.4 | 67 | 2.09E+05 | 4.04E−03 | 1.93E−08 | 2.9 |
| mAb27404 | 453 + 4 | 67 | 2.40E+05 | 1.83E−03 | 7.63E−09 | 6 |
| mAb27242 | 441 + 1.1 | 82 | 1.44E+05 | 8.30E−04 | 5.77E−09 | 14 |
| mAb27243 | 412 + 1.8 | 71 | 1.23E+05 | 1.16E−03 | 9.41E−09 | 10 |
| mAb27245 | 448 + 2.2 | 66 | 5.39E+04 | 1.73E−03 | 3.22E−08 | 7 |
| mAb27241 | 430 + 0.9 | 66 | 4.32E+04 | 8.52E−04 | 1.97E−08 | 14 |
| mAb29266 | 418 + 3.8 | 38 | 1.56E+05 | 2.99E−02 | 1.91E−07 | 0.39 |
| mAb27400 | 430 + 2.9 | 15 | 9.71E+03 | 4.76E−04 | 4.90E−08 | 24 |
| mAb22125 | 892 + 3.1 | 25 | IC # | IC # | IC # | IC # |
| mAb22124 | 831 + 1.3 | 14 | IC # | IC # | IC # | IC # |
| mAb22168P | 842 + 0.8 | 27 | IC # | IC # | IC # | IC # |
| mAb29233 | 453 + 7.4 | 3 | NB* | NB* | NB* | NB* |
| mAb29237 | 330 + 23 | 1 | NB* | NB* | NB* | NB* |
| mAb29256 | 371 + 5.1 | 0 | NB* | NB* | NB* | NB* |
| mAb29261 | 405 + 3.9 | 5 | NB* | NB* | NB* | NB* |
| mAb22115 | 835 + 1.9 | 86 | 7.04E+04 | 3.08E−02 | 4.38E−07 | 0.37 |
| Isotype | 879 + 1.3 | 0 | NB* | NB* | NB* | NB* |
| Control mAb | | | | | | |

*NB indicates that no binding was observed under the current experimental conditions
#IC indicates that the binding data was inconclusive

TABLE 6
Binding kinetics parameters of hACVR1-mFc binding to anti-ACVR1
monoclonal antibodies at 25° C.

| | mAb Capture | 100 nM Ag | kₐ | k_{d} | K_{D} | t_{1/2} |
|---|---|---|---|---|---|---|
| mAb captured | Level (RU) | Bound (RU) | (1/Ms) | (1/s) | (M) | (min) |
| mAb29257 | 350 + 2 | 157 | 1.29E+05 | 1.21E−04 | 9.36E−10 | 95 |
| mAb27396 | 351 + 1.7 | 170 | 3.24E+05 | 4.93E−04 | 1.52E−09 | 23 |
| mAb29226 | 341 + 0.9 | 188 | 2.10E+05 | 1.61E−04 | 7.67E−10 | 72 |
| mAb27405 | 363 + 1.9 | 142 | 1.06E+05 | 2.01E−04 | 1.90E−09 | 57 |
| mAb27247 | 346 + 0.7 | 186 | 2.29E+05 | 1.80E−04 | 7.87E−10 | 64 |
| mAb27404 | 363 + 1.1 | 160 | 1.98E+05 | 8.76E−05 | 4.44E−10 | 132 |
| mAb27242 | 362 + 1.5 | 199 | 1.44E+05 | 2.90E−05 | 2.02E−10 | 398 |
| mAb27243 | 349 + 4.7 | 170 | 1.13E+05 | 3.63E−05 | 3.18E−10 | 318 |
| mAb27245 | 362 + 2 | 145 | 3.81E+04 | 5.06E−05 | 1.34E−09 | 228 |
| mAb27241 | 345 + 2.1 | 135 | 3.49E+04 | 3.20E−05 | 9.18E−10 | 361 |
| mAb29266 | 353 + 1.7 | 178 | 1.14E+05 | 6.20E−04 | 5.43E−09 | 19 |
| mAb27400 | 348 + 1.4 | 24 | 1.19E+04 | 1.42E−04 | 1.19E−08 | 81 |
| mAb22125 | 685 + 3.2 | 268 | 6.52E+05 | 8.02E−03 | 1.23E−08 | 1.4 |
| mAb22124 | 637 + 1.2 | 172 | 7.39E+05 | 4.09E−02 | 5.54E−08 | 0.28 |
| mAb22168 | 645 + 0.7 | 264 | 8.37E+05 | 9.28E−03 | 1.11E−08 | 1.2 |
| mAb29233 | 368 + 0.6 | 2 | NB* | NB* | NB* | NB* |
| mAb29237 | 279 + 2.9 | −2 | NB* | NB* | NB* | NB* |
| mAb29256 | 315 + 1.9 | −1 | NB* | NB* | NB* | NB* |
| mAb29261 | 334 + 2.4 | 0 | NB* | NB* | NB* | NB* |
| mAb22115 | 629 + 1.1 | 264 | 6.69E+04 | 7.65E−04 | 1.14E−08 | 15 |
| Isotype Control | 638 + 0.9 | −2 | NB* | NB* | NB* | NB* |
| mAb | | | | | | |

*NB indicates that no binding was observed under the current experimental conditions.

TABLE 7
Binding kinetics parameters of hACVR1-mFc
binding to anti-ACVR1 monoclonal antibodies at 37° C.

| | mAb Capture | 100 nM Ag | kₐ | k_{d} | K_{D} | t_{1/2} |
|---|---|---|---|---|---|---|
| mAb captured | Level (RU) | Bound (RU) | (1/Ms) | (1/s) | (M) | (min) |
| mAb29257 | 425 + 1.9 | 201 | 3.14E+05 | 4.95E−04 | 1.58E−09 | 23 |
| mAb27396 | 448 + 3.5 | 197 | 4.54E+05 | 1.96E−03 | 4.31E−09 | 6 |
| mAb29226 | 387 + 1.5 | 212 | 3.40E+05 | 5.21E−04 | 1.53E−09 | 22 |
| mAb27405 | 434 + 1.9 | 185 | 2.33E+05 | 7.42E−04 | 3.18E−09 | 16 |
| mAb27247 | 393 + 2.3 | 219 | 3.71E+05 | 5.15E−04 | 1.39E−09 | 22 |
| mAb27404 | 449 + 1.2 | 216 | 3.03E+05 | 3.38E−04 | 1.11E−09 | 34 |
| mAb27242 | 437 + 1 | 250 | 2.82E+05 | 1.89E−04 | 6.69E−10 | 61 |
| mAb27243 | 409 + 1.3 | 228 | 2.36E+05 | 2.59E−04 | 1.10E−09 | 45 |
| mAb27245 | 444 + 2 | 222 | 9.17E+04 | 3.35E−04 | 3.65E−09 | 34 |
| mAb27241 | 426 + 1.7 | 216 | 8.22E+04 | 2.01E−04 | 2.44E−09 | 57 |
| mAb29266 | 411 + 2.8 | 205 | 2.35E+05 | 9.11E−04 | 3.88E−09 | 13 |
| mAb27400 | 418 + 2.5 | 68 | 1.27E+04 | 1.63E−04 | 1.28E−08 | 71 |
| mAb22125 | 885 + 2.4 | 243 | 7.29E+05 | 5.77E−02 | 7.92E−08 | 0.20 |
| mAb22124 | 825 + 2.7 | 158 | 8.23E+05 | 1.19E−01 | 1.45E−07 | 0.10 |
| mAb22168 | 838 + 0.8 | 278 | 8.12E+05 | 4.58E−02 | 5.64E−08 | 0.25 |
| mAb29233 | 445 + 1 | 3 | NB* | NB* | NB* | NB* |
| mAb29237 | 263 + 6 | 5 | NB* | NB* | NB* | NB* |
| mAb29256 | 350 + 2.6 | 3 | NB* | NB* | NB* | NB* |
| mAb29261 | 394 + 5.4 | 11 | IC # | IC # | IC # | IC # |
| mAb22115 | 830 + 1.5 | 317 | 8.33E+04 | 2.50E−03 | 3.01E−08 | 4.6 |
| Isotype | 875 + 1.1 | −1 | NB* | NB* | NB* | NB* |
| Control mAb | | | | | | |

*NB indicates that no binding was observed under the current experimental conditions.
#IC indicates that the binding data was inconclusive

TABLE 8
Binding kinetics parameters of mACVR1-mmh binding to anti-ACVR1
monoclonal antibodies at 25° C.

| | | 900 nM or | | | | |
|---|---|---|---|---|---|---|
| | mAb Capture | 100 nM Ag | kₐ | k_{d} | K_{D} | t_{1/2} |
| mAb captured | Level (RU) | Bound (RU) | (1/Ms) | (1/s) | (M) | (min) |
| mAb29257 | 353 + 1.3 | −2 | NB* | NB* | NB* | NB* |
| mAb27396 | 353 + 1.4 | 1 | NB* | NB* | NB* | NB* |
| mAb29226 | 342 + 0.5 | 2 | NB* | NB | NB* | NB* |
| mAb27405 | 364 + 1.2 | 0 | NB* | NB* | NB* | NB* |
| mAb27247 | 347 + 0.8 | 3 | NB* | NB* | NB* | NB* |
| mAb27404 | 364 + 1.1 | 1 | NB* | NB* | NB* | NB* |
| mAb27242 | 362 + 1.8 | 6 | 2.38E+05 | 1.44E−01 | 6.02E−07 | 0.08 |
| mAb27243 | 349 + 2 | 6 | 1.48E+05 | 1.31E−01 | 8.83E−07 | 0.09 |
| mAb27245 | 363 + 1 | 1 | NB* | NB* | NB* | NB* |
| mAb27241 | 347 + 0.8 | 1 | NB* | NB* | NB* | NB* |
| mAb29266 | 353 + 1.4 | 5 | IC # | IC # | IC # | IC # |
| mAb27400 | 349 + 1.4 | −3 | NB* | NB* | NB* | NB* |
| mAb22125 | 686 + 2.8 | 41 | 6.88E+05 | 5.75E−01 | 8.36E−07 | 0.02 |
| mAb22124 | 640 + 1 | 18 | 7.08E+05 | 1.50E+00 | 2.13E−06 | 0.01 |
| mAb22168 | 647 + 0.3 | 33 | 1.25E+06 | 1.08E+00 | 8.65E−07 | 0.01 |
| mAb29233 | 369 + 0.7 | 1 | NB* | NB* | NB* | NB* |
| mAb29237 | 292 + 5.1 | −3 | NB* | NB* | NB* | NB* |
| mAb29256 | 323 + 3.3 | −3 | NB* | NB* | NB* | NB* |
| mAb29261 | 338 + 1.4 | −1 | NB* | NB* | NB* | NB* |
| mAb22115 | 629 + 1.2 | 94 | 2.80E+04 | 4.78E−03 | 1.71E−07 | 2.4 |
| Isotype Control | 638 + 0.4 | −2 | NB* | NB* | NB* | NB* |
| mAb | | | | | | |

*NB indicates that no binding was observed under the current experimental conditions.
#IC indicates that the binding data was inconclusive.

TABLE 9
Binding kinetics parameters of mACVR1-mmh binding to anti-ACVR1
monoclonal antibodies at 37° C.

| | | 900 nM or | | | | |
|---|---|---|---|---|---|---|
| | mAb Capture | 100 nM Ag | kₐ | k_{d} | K_{D} | t_{1/2} |
| mAb captured | Level (RU) | Bound (RU) | (1/Ms) | (1/s) | (M) | (min) |
| mAb29257 | 430 + 3 | 2 | NB* | NB* | NB* | NB* |
| mAb27396 | 451 + 4.4 | 4 | NB* | NB* | NB* | NB* |
| mAb29226 | 392 + 1.9 | 2 | NB* | NB* | NB* | NB* |
| mAb27405 | 438 + 1.1 | 2 | NB* | NB* | NB* | NB* |
| mAb27247 | 398 + 1.4 | 4 | NB* | NB* | NB* | NB* |
| mAb27404 | 452 + 1.9 | 4 | NB* | NB* | NB* | NB* |
| mAb27242 | 439 + 1.2 | 4 | NB* | NB* | NB* | NB* |
| mAb27243 | 410 + 1.8 | 5 | IC # | IC # | IC # | IC # |
| mAb27245 | 446 + 2.6 | 2 | NB* | NB* | NB* | NB* |
| mAb27241 | 428 + 1.1 | 2 | NB* | NB* | NB* | NB* |
| mAb29266 | 407 + 17.4 | 5 | NB* | NB* | NB* | NB* |
| mAb27400 | 423 + 3 | 2 | NB* | NB* | NB* | NB* |
| mAb22125 | 887 + 1.6 | 23 | IC # | IC # | IC # | IC # |
| mAb22124 | 828 + 1.1 | 14 | IC # | IC # | IC # | IC # |
| mAb22168 | 840 + 0.8 | 23 | IC # | IC # | IC # | IC # |
| mAb29233 | 447 + 1.1 | 1 | NB* | NB* | NB* | NB* |
| mAb29237 | 287 + 9 | −1 | NB* | NB* | NB* | NB* |
| mAb29256 | 358 + 3.4 | 0 | NB* | NB* | NB* | NB* |
| mAb29261 | 399 + 5 | 2 | NB* | NB* | NB* | NB* |
| mAb22115 | 834 + 1.4 | 85 | 5.89E+04 | 2.97E−02 | 5.04E−07 | 0.39 |
| Isotype Control | 877 + 1.8 | 0 | NB* | NB* | NB* | NB* |
| mAb | | | | | | |

*NB indicates that no binding was observed under the current experimental conditions.
#IC indicates that the binding data was inconclusive

At 25° C., anti-ACVR1 monoclonal antibodies that bound to hACVR1-MMH had K_{D} values ranging from 1.56 nM to 1.97 μM, as shown in Table 4. At 37° C., anti-ACVR1 monoclonal antibodies that bound to hACVR1-MMH had K_{D} values ranging from 5.77 nM to 438 nM, as shown in Table 5.

At 25° C., anti-ACVR1 monoclonal antibodies that bound to hACVR1-mFc had K_{D} values ranging from 0.20 nM to 55.4 nM, as shown in Table 6. At 37° C., anti-ACVR1 monoclonal antibodies that bound to hACVR1-mFc had K_{D} values ranging from 0.67 nM to 145 nM, as shown in Table 7.

At 25° C., anti-ACVR1 monoclonal antibodies that bound to mACVR1-MMH had K_{D} values ranging from 171 nM to 2.13 μM, as shown in Table 8. At 37° C., only one anti-ACVR1 monoclonal antibody bound to mACVR1-MMH with a K_{D} value of 504 nM, as shown in Table 9.

### Example 4: Cross-Competition Between Different Anti-ACVR1 Monoclonal Antibodies

### Experimental Procedure

Binding competition within a panel of anti-ACVR1 monoclonal antibodies was determined using a real time, label-free bio-layer interferometry assay on the Octet HTX biosensor platform (Pall ForteBio Corp.). The entire experiment was performed at 25° C. in 10 mM HEPES, 150 mM NaCI, 3 mM EDTA, and 0.05% v/v Surfactant Tween-20, 1 mg/mL BSA, pH 7.4 (HBS-EBT) buffer with the plate shaking at the speed of 1000 rpm. To assess whether two antibodies are able to compete with one another for binding to their respective epitopes, human ACVR1 extracellular domain expressed with a C-terminal myc-myc-hexahistidine tag (hACVR1-MMH; SEQ ID NO: 338) was first captured by dipping anti-His antibody coated Octet biosensor tips (Fortebio Inc, #18-5079) by submerging the biosensor tips in wells containing 10 μg/mL hACVR1-MMH for 40 seconds. The antigen captured biosensor tips were then saturated with the first anti-ACVR1 monoclonal antibody (referred to as mAb-1) by dipping into wells containing 50 μg/mL solution of mAb-1 for 4 minutes. The biosensor tips were then dipped into wells containing 50 μg/mL solution of second anti-ACVR1 monoclonal antibody (referred to as mAb-2) for 3 minutes. The biosensor tips were washed in HBS-EBT buffer between every step of the experiment. The real-time binding response was monitored over the entire duration of the experiment and the binding response at the end of every step was recorded. The response of mAb-2 binding to hACVR1-MMH complexed with mAb-1 was compared and competitive/non-competitive behavior of different anti-ACVR1 monoclonal antibodies was determined as shown in Table 10.

### Results

TABLE 10
Cross-competition between
anti-ACVR1 monoclonal antibodies

| mAb-1 | mAb-2 competing with mAb-1 |
|---|---|
| mAb29257 | mAb27396 |
| | mAb27242 |
| | mAb27243 |
| | mAb27241 |
| | mAb27245 |
| | mAb27247 |
| | mAb27404 |
| | mAb27405 |
| mAb27396 | mAb29257 |
| | mAb27242 |
| | mAb27243 |
| | mAb27241 |
| | mAb27245 |
| | mAb27247 |
| | mAb27404 |
| | mAb27405 |
| mAb27242 | mAb29257 |
| | mAb27396 |
| | mAb27243 |
| | mAb27241 |
| | mAb27245 |
| | mAb27247 |
| | mAb27404 |
| | mAb27405 |
| mAb27243 | mAb29257 |
| | mAb27396 |
| | mAb27242 |
| | mAb27241 |
| | mAb27245 |
| | mAb27247 |
| | mAb27404 |
| | mAb27405 |
| mAb27241 | mAb29257 |
| | mAb27396 |
| | mAb27242 |
| | mAb27243 |
| | mAb27245 |
| | mAb27247 |
| | mAb27404 |
| | mAb27405 |
| mAb27245 | mAb29257 |
| | mAb27396 |
| | mAb27242 |
| | mAb27243 |
| | mAb27241 |
| | mAb27247 |
| | mAb27404 |
| | mAb27405 |
| mAb27247 | mAb29257 |
| | mAb27396 |
| | mAb27242 |
| | mAb27243 |
| | mAb27241 |
| | mAb27245 |
| | mAb27404 |
| | mAb27405 |
| mAb27404 | mAb29257 |
| | mAb27396 |
| | mAb27242 |
| | mAb27243 |
| | mAb27241 |
| | mAb27245 |
| | mAb27247 |
| | mAb27405 |
| mAb27405 | mAb29257 |
| | mAb27396 |
| | mAb27242 |
| | mAb27243 |
| | mAb27241 |
| | mAb27245 |
| | mAb27247 |
| | mAb27404 |

Table 10 shows the cross-competition between selected anti-ACVR1 antibodies.

### Example 5: Cell Binding by Flow Cytometry with HEK293/hACVR1-wt and HEK293/hACVR1-R206H Cells

In order to assess cell binding by anti-hACVR1 antibodies two cell lines were generated to stably over-express full length hACVR1 in HEK293 cells along with a BMP-response element fused to firefly luciferase reporter (BRE-Luc). One cell line contained the wild type version of hACVR1 (amino acids 1-509 of accession #Q04771), and was named HEK293/BRE-luc/hACVR1-wild type. It is hereafter referred to as HEK293/hACVR1-wt. The other line contained hACVR1 (R206H). A single clone of this cell line was isolated, and the resulting cell line was named HEK293/BRE-luc/hACVR1-R206H-clone H2. It is hereafter referred to as HEK293/hACVR1-R206H.

To assess binding of the anti-ACVR1 antibodies of the invention to the receptor expressed on the cell surface, either 66.6 nM or 70 nM of the antibodies were incubated with 0.5×10⁶ cells/well at 4° C. for 30 minutes in PBS (without calcium and magnesium) containing 2% FBS. After incubation with primary antibodies, the cells were stained with 3.2 μg/mL of Alexa Fluor®-647 conjugated secondary antibody (Jackson ImmunoResearch Laboratories Inc., anti-human #109-607-003) at 4° C. for 25 or 30 minutes. Cells were fixed using BD CytoFix™ (Becton Dickinson, #554655) and analyzed on either Hypercyt® or IQue® Flow Cytometers (Intellicyt®). Unstained and secondary antibody alone controls were also tested for all cell lines. The results were analyzed using ForeCyt® (IntelliCyt®) software to determine the geometric means of fluorescence for viable cells and binding ratios were calculated by normalizing the geometric mean value of the test condition by the geometric mean value of the corresponding unstained cells.

TABLE 11
Binding of anti-hACVR1 antibodies to HEK293/BRE-luc/hACVR1 cells

| | MFI-Normalized to Unstained Control | | | | |
|---|---|---|---|---|---|
| | 66.6 nM antibody | | | 70 nM antibody | |
| | HEK293 | HEK293/ | HEK293/ | HEK293 | HEK293/ |
| mAb PID | Parental | hACVR1-wt | hACVR1-R206H | Parental | hACVR1-R206H |
| mAb22115 | 6 | 4 | 4 | Not | Not Tested |
| | | | | Tested | |
| mAb22124 | 2 | 41 | 116 | Not | Not Tested |
| | | | | Tested | |
| mAb22125 | 1 | 183 | 235 | Not | Not Tested |
| | | | | Tested | |
| mAb22168 | 1 | 116 | 213 | Not | Not Tested |
| | | | | Tested | |
| mAb29226 | Not Tested | Not Tested | Not Tested | 23 | 1900 |
| mAb29233 | Not Tested | Not Tested | Not Tested | 3 | 5 |
| mAb29237 | Not Tested | Not Tested | Not Tested | 9 | 10 |
| mAb29256P | Not Tested | Not Tested | Not Tested | 3 | 3 |
| mAb29257 | Not Tested | Not Tested | Not Tested | 26 | 1662 |
| mAb29261 | Not Tested | Not Tested | Not Tested | 2 | 2 |
| mAb29266 | Not Tested | Not Tested | Not Tested | 13 | 1642 |
| mAb27396 | Not Tested | Not Tested | Not Tested | 2 | 954 |
| mAb27241 | Not Tested | Not Tested | Not Tested | 19 | 1165 |
| mAb27242 | Not Tested | Not Tested | Not Tested | 23 | 1734 |
| mAb27243 | Not Tested | Not Tested | Not Tested | 18 | 1570 |
| mAb27245 | Not Tested | Not Tested | Not Tested | 21 | 1662 |
| mAb27247 | Not Tested | Not Tested | Not Tested | 7 | 1659 |
| mAb27404 | Not Tested | Not Tested | Not Tested | 18 | 1872 |
| mAb27405 | Not Tested | Not Tested | Not Tested | 11 | 1563 |
| mAb27400 | Not Tested | Not Tested | Not Tested | 7 | 840 |
| hIgG4 | 1 | 3 | 1 | 1 | 3 |
| Isotype | | | | | |
| Control | | | | | |
| Anti-Human | 1 | 1 | 1 | 1 | 1 |
| 2″ alone | | | | | |

As shown in Table 11, four of the 20 anti-hACVR1 antibodies of the invention showed binding to HEK293/hACVR1-wt cells with binding ratios ranging from 4 to 183-fold. All twenty anti-hACVR1 antibodies of the invention were tested in binding to HEK293/hACVR1-R206H cells and they showed binding to cells with binding ratios ranging from 2 to 1900-fold. The anti-hACVR1 antibodies of the invention demonstrated binding to the HEK293 parental cells, with binding ratios 1 to 26-fold. The isotype control antibodies and secondary antibodies alone samples demonstrated binding ratios ranging from 1 to 3-fold.

### Example 6: Functional Inhibition of ACVR1 in Cell-Based Bioassay with HEK293/BRE-luc/hACVR1-R206H-clone H2 Cells, Activated by hBMP7 or hActivin A

Activin A receptor type I, ACVR1 (also known as ActRI, ACVR1A, or Alk2), is a single-pass transmembrane receptor, and a member of the type I BMP receptor of the TGF-β receptor super family. Upon ligand binding, ACVR1 together with a type II receptor initiates a downstream signaling cascade leading to activation of receptor specific R-SMAD protein (SMAD1, SMAD5, or SMAD8) and collaborating SMAD, SMAD4, and leads to transcriptional regulation of genes (Massagué J, TGF-beta Signal Transduction, Annu. Rev. Biochem. 1998. 67:753-91, PMID: 9759503; Massagué et al. Smad transcription factors, Genes Dev. 2005 19: 2783-2810, PMID: 16322555). In order to assess anti-ACVR1 antibody inhibition of ACVR1 (R206H), the mutation found in FOP (Shore et al., Nat Genet. 2006 May;38(5):525-7. Epub 2006 Apr 23. PMID: 16642017), a bioassay was established in HEK293 cells (human embryonic kidney, ATCC). HEK293 cells endogenously express ACVR1, the necessary Type II receptors, SMAD proteins, and other components that form a functional BMP signaling pathway. To drive the signaling through ACVR1, a cell line was generated to stably overexpress full length human ACVR1 (amino acids 1-509, R206H, of accession #Q04771), along with a BMP-response element fused to firefly luciferase reporter (BRE-Luc). A single clone of the cell line was isolated, and the resulting cell line was named HEK293/BRE-luc/hACVR1-R206H-clone H2. It is hereafter referred to as HEK293/BRE-luc/hACVR1-R206H.

For the bioassay, HEK293/BRE-luc/hACVR1-R206H cells were plated at 10,000 cells/well in a 96-well plate in assay buffer (DMEM High Glucose+10% FBS+Pen/Strep/L-Glutamine) and incubated for 5 hours at 37° C. in 5% CO₂. Following the 5 hour incubation, anti-ACVR1 antibodies or an isotype control antibody that were serially diluted in assay buffer from either 300 nM to 73.2 pM or 173.3 nM to 42.3 pM (plus a sample containing buffer alone without test molecule) were added to the cells and incubated at 25° C. for 30 minutes. After 30 minutes, either 3 nM human Activin A (hActivin A, R&D System 338-AC), 2 nM human Bone Morphogenetic Protein 7 (hBMP7, R&D System 354-BP/C) or 3 nM hBMP7 were added to cells. To obtain a dose dependent activation by the ligands, hActivin A or hBMP7 were serially diluted from either 200 nM to 3.4 pM or 100 nM to 1.7 pM in assay buffer (plus a sample containing buffer alone without test molecule) and added to cells not treated with antibodies. After overnight incubation at 37° C. in 5% CO₂, luciferase activity was measured with OneGlo™ reagent (Promega, #E6031) and VictorX or Envision plate readers (Perkin Elmer). The results were analyzed using nonlinear regression (4-parameter logistics) with Prism software (GraphPad) to obtain EC₅₀ and IC₅₀ values. The percentage of inhibition was calculated with the RLU values by using the following equation:

$$\\ \% Inhibition = 100 \times \frac{R L {U}_{Baseline} - R L {U}_{Inhibition}}{R L {U}_{Baseline} - R L {U}_{Background}}$$

In this equation “RLU_{Baseline}” is the luminescence value from the cells treated constant amount of ligand (hActivin A or hBMP7) without antibodies, “RLU_{Inhibition}” is the luminescence value with maximum concentration of a particular antibody with a particular concentration of ligand, and “RLU_{Background}” is the luminescence value from cells without any ligands or antibodies.

Twenty anti-human ACVR1 antibodies of the invention were tested for their ability to inhibit activation of HEK293/BRE-luc/hACVR1-R206H cells. Results are shown in Table 12.

TABLE 12
Inhibition of anti-hACVR1 antibodies in presence of hACVR1 ligands in
HEK293/BRE-luc/hACVR1-R206H cells

| Ligand | | | | | | |
|---|---|---|---|---|---|---|
| EC₅₀ [M] | hActivin A | | hBMP7 | | | |
| Constant | 3.33E−10 | | 2.97E−10 | | 1.22E−09 | |
| Ligand | 3 nM hActivin A | | 3 nM hBMP7 | | 2 nM hBMP7 | |
| mAb PID | IC₅₀ [M] | % Inhibition | IC₅₀ [M] | % Inhibition | IC₅₀ [M] | % Inhibition |
| mAb22115 | No | No | No | No | Not Tested | Not Tested |
| | Inhibition | Inhibition | Inhibition | Inhibition | | |
| mAb22124 | 1.8E−08 | 51 | >1.0E−07 | 46 | Not Tested | Not Tested |
| mAb22125 | 1.9E−08 | 78 | 1.6E−08 | 51 | Not Tested | Not Tested |
| mAb22168 | 2.3E−08 | 75 | >1.0E−07 | 52 | Not Tested | Not Tested |
| mAb29226 | Not Tested | Not Tested | Not Tested | Not Tested | 7.3E−10 | 70 |
| mAb29233 | Not Tested | Not Tested | Not Tested | Not Tested | No | No |
| | | | | | Inhibition | Inhibition |
| mAb29237 | Not Tested | Not Tested | Not Tested | Not Tested | No | No |
| | | | | | Inhibition | Inhibition |
| mAb29256 | Not Tested | Not Tested | Not Tested | Not Tested | No | No |
| | | | | | Inhibition | Inhibition |
| mAb29257 | Not Tested | Not Tested | Not Tested | Not Tested | 1.4E−10 | 62 |
| mAb29261 | Not Tested | Not Tested | Not Tested | Not Tested | No | No |
| | | | | | Inhibition | Inhibition |
| mAb29266 | Not Tested | Not Tested | Not Tested | Not Tested | 1.9E−09 | 93 |
| mAb27396 | Not Tested | Not Tested | Not Tested | Not Tested | 5.8E−10 | 90 |
| mAb27241 | Not Tested | Not Tested | Not Tested | Not Tested | 1.8E−09 | 110 |
| mAb27242 | Not Tested | Not Tested | Not Tested | Not Tested | 1.2E−09 | 109 |
| mAb27243 | Not Tested | Not Tested | Not Tested | Not Tested | 1.3E−09 | 112 |
| mAb27245 | Not Tested | Not Tested | Not Tested | Not Tested | 1.5E−09 | 110 |
| mAb27247 | Not Tested | Not Tested | Not Tested | Not Tested | 7.6E−10 | 111 |
| mAb27404 | Not Tested | Not Tested | Not Tested | Not Tested | 8.8E−10 | 109 |
| mAb27405 | Not Tested | Not Tested | Not Tested | Not Tested | 7.6E−10 | 107 |
| mAb27400 | Not Tested | Not Tested | Not Tested | Not Tested | 2.0E−09 | 96 |
| hIgG4 Isotype | No | No | No | No | No | No |
| (REGN1945) | Inhibition | Inhibition | Inhibition | Inhibition | Inhibition | Inhibition |

As shown in Table 12, ten of the antibodies of the invention, showed at least 90% inhibition of 2 nM hBMP7, with IC₅₀ values for the inhibiting antibodies ranging from 580 pM to 2.0 nM. Five of the antibodies of the invention, showed between 46% and 78% inhibition of either 3 nM hActivin A, 2 nM or 3 nM hBMP7, with IC₅₀ values for the inhibiting antibodies ranging from 140 pM to >100 nM. Five antibodies of the invention did not show inhibition of any of ligands tested. An isotype control antibody did not demonstrate any measurable inhibition of HEK293/BRE-luc/hACVR1-R206H cells activated by either hActivin A or hBMP7. The ligands activated HEK293/BRE-luc/hACVR1-R206H cells with EC₅₀ values of 297 pM or 1.22 nM for hBMP7, and 333 pM for hActivin A.

### Example 7: Anti-ACVR1 Antibody Testing in vivo; Serum Analysis of Acvr1^{hu/hu} mice for Hepcidin and Iron Levels

Hepcidin and iron levels were tested in Acvr1^{hu/hu} mice after treatment with anti-ACVR1 antibodies mAb27242; mAb27243; mAb27247; and hIgG4 isotype control antibody (REGN1945).

BMP6 mediated activation of ACVR1 directly activates transcription of Hamp, the gene that encodes hepcidin. Hepcidin is a negative regulator of iron levels by causing internalization of ferroportin (slc40a1), the only known iron exporter. Inhibition of the BMP6-ACVR1 signaling cascade leads to decreased Hamp transcription, resulting in decreased circulating levels of hepcidin. A reduction of circulating hepcidin results in increased ferroportin levels, which allows increased uptake of iron from the small intestines, thereby increasing circulating iron levels.

Therefore, to determine the effects of anti-ACVR 1 antibodies of the invention on serum hepcidin and iron, an in vivo experiment in mice was performed. For the experiment, mice expressing human ACVR1 in place of the mouse allele (referred to as Acvr1^{huhu} mice) were utilized. Forty-two female Acvr1^{hu/hu} mice (12-15 weeks old) were dosed with 10 mg/kg of either isotype control, mAb27242, mAb27243 or mAb27247 on Days 1 and 5 of the experiment. Mice were sacrificed for serum collection at Day 8. Serum was analyzed for Hepcidin protein levels using the Hepcidin-Murine Complete ELISA (Intrinsic Lifesciences, Cat# HMC-001) and iron levels using a QuantiChrom Iron Assay Kit (BioAssay Systems, Cat# DIFE-250). Results are shown in Table 13.

TABLE 13
Serum Hepcidin and Serum Iron levels in Acvr1^{hu/hu} mice

| | Serum Hepcidin | | Serum Iron | |
|---|---|---|---|---|
| | | Standard | | Standard |
| Antibody tested | Mean | deviation | Mean | deviation |
| Isotype control | 288.3 | 71.92 | 206.6 | 64.74 |
| mAb27242 | 140.0 | 78.86 | 462.1 | 70.77 |
| mAb27243 | 123.7 | 34.58 | 513.1 | 140.0 |
| mAb27247 | 220.1 | 58.66 | 332.1 | 159.7 |

As shown in Table 13, ACVR1 antibodies of the invention, mAb27242 and mAb27243, decreased serum hepcidin and increased serum iron levels in Acvr1^{hu/hu} mice, whereas mAb27247 showed no effect on serum hepcidin or serum iron levels in Acvr1^{huhu} mice. This indicates that mAb27242 and mAb27243 can inhibit wild type ACVR1 signaling.

### Example 8: Anti-ACVR1 Antibody Testing in vivo; Post-Traumatic Heterotopic Ossification Model

The present study evaluated effects of an anti-ACVR1 antibody of the invention mAb27242 and an anti-activin A antibody in an in vivo post-traumatic HO model in mice.

Heterotopic ossification (HO), the formation of ectopic bone in soft tissues, occurs in two primary forms: post-traumatic HO (tHO) typically found in patients who have experienced musculoskeletal or neurogenic injury and the genetically driven fibrodysplasia ossificans progressive (FOP) downstream to a specific point mutation known as R206H in the ACVR1 receptor. Both diseases undergo a process of endochondral ossification in the formation of ectopic bone.

The principle management for HO remains surgical excision that is often complicated by recurrence, nearly universally so in FOP. While both post-traumatic and FOP varieties of HO have been demonstrated to reflect an aberrancy in inflammation that triggers endochondral ossification, the antecedent signals for this convergent programming seem distinct within the existing literature. In both varieties, pathology appears dependent on the signaling from a specific subset of receptors sensitive to ligands of the transforming growth factor beta (TGFB) superfamily including Alk2/ACVR1, Alk3/BMPR1A, Alk4/ACVR1B, Alk5/TGFBRI, Alk6/BMPR1B and Alk7/ACVR1C.

Activin A is found in FOP fibroblasts. Sequestration of activin A in validated mouse models of FOP have demonstrated near eradication of subsequent lesions. Muscle injury in a mouse model of FOP (Acvr1[R206H]) results in HO that can be completely abrogated using an activin A blocking antibody (Hatsell et al. Sci Transl Med. Sep 2 2015; 7(303):303ra137). Effective attenuation of FOP HO with pharmacologic inhibition of activin A via an anti-activin A neutralizing antibody REGN2477 has also been demonstrated (Upadhyay et al., 2017, J Bone Mineral Res 32(12):2489-2499).

However, recent literature has identified a contrast between tHO and FOP, namely the ACVR1 gene conferring a net gain-of-function and novel activation by activin A as the primary driving force behind FOP lesions.

The present experiment evaluated the effect of an anti-ACVR1 antibody of the invention and an anti-activin A antibody in an in vivo post-traumatic HO model in mice. Specifically, post-traumatic heterotopic ossification was measured by microCT analysis in mice after treatment with anti-ACVR1 antibody mAb27242.

### Recombinant Proteins and Antibody Dosing of Mice

A human Acvr1 antibody (mAb27242 according to the present invention and a neutralizing antibody generated against human activin A (US Patent Application 20150037339) were employed. ALK3-Fc was also employed in post-traumatic HO formation to investigate potential inhibitory impact of inhibiting several of the osteogenic BMPs. Alk3-Fc was generated in house, in CHO cells and purified. Alk3-Fc consists of the extracellular domain of Alk3 (Swiss Prot# P27037 Q24-R152) linked to the human IgG1 Fc domain (D104-K330).

For treatment studies mice were separated to ensure age matching across groups, treatments were initiated on the same day as injury. Mice (n=15/group) were injected subcutaneously (s.c.) weekly with 25 mg/kg of an activin A blocking antibody, or isotype-control antibody. For the second experiment mice (n=12/group) were injected s.c. with 10 mg/kg of the Acvr1 blocking antibody, Alk3-Fc or an isotype control antibody. HO formation was monitored by in vivo microCT imaging over a period of at least 13 weeks.

### Burn/Tenotomy Injury Model

Mice evaluated for ectopic bone were wild type (WT) C57BL/6J mice (Jackson Laboratories). Briefly, WT mice were injected with tamoxifen for 5 days @40 mg/kg i.p., to initiate model. All mice received presurgical analgesia consisting of 0.06 mg·kg−1 buprenorphine for 48 h, followed by anesthesia with inhaled isoflurane, and close postoperative monitoring with analgesic administration. Mice received 30% total body surface area partial-thickness burn on a shaved dorsum followed by transection of the left Achilles tendon. Dorsal burn was induced using a metal block heated to 60° C.in a water bath and applied to the dorsum for 18 s continuously. HO anlagen was observed by week 3 with mature bone formation visible by microCT by 9 weeks.

### Results

Acvr1 blocking antibodies or Alk3-Fc attenuated HO in the post-traumatic HO model in mice; however, inhibition of activin A does not alter HO formation.

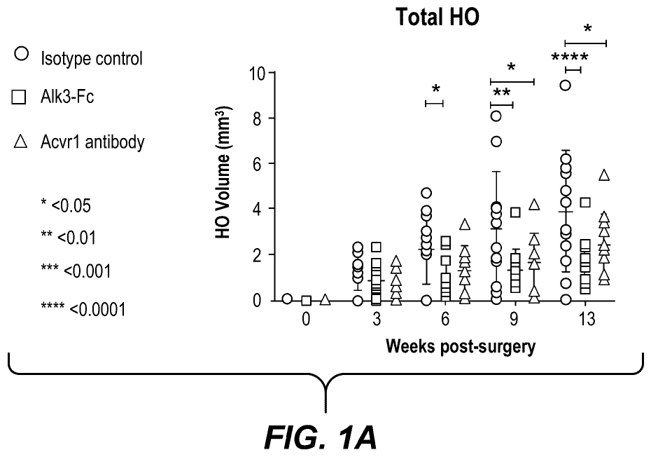

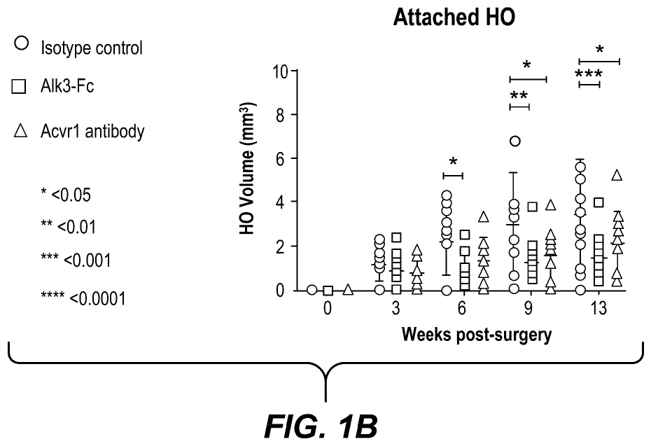

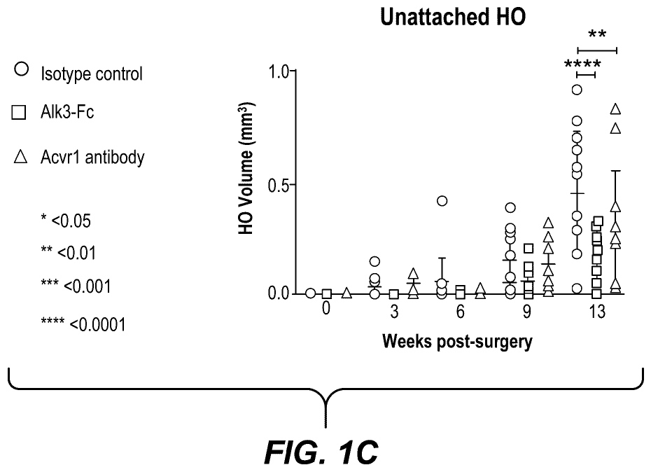

Mice were administered either isotype control (n=12) or anti-ACVR (n=12) antibodies or Alk3-Fc (n=12) starting concurrently with induction of injury in the tHO model. FIGS. 1A-C show results of microCT analysis in mice that were administered either an isotype control antibody (circles, n=12), ALK3-Fc (squares, n=12) or an Acvr1 antibody (triangles, n=12) (mAb27242) starting concurrently with induction of injury. Total HO (FIG. 1A), attached HO (FIG. 1B), and unattached HO (FIG. 1C) volumes were measured by microCT analysis at 3, 6, 9 and 13 weeks post injury.

In wild type mice induced with burn/tenotomy injury, inhibition of ACVR1 using a blocking antibody decreased HO formation by 40% (3.92 mm³ vs 2.4 mm³ total HO at week 13) demonstrating that at least some of the BMP signal responsible for HO formation and growth was transmitted through ACVR1. (FIGS. 1A-C). Acvr1 blocking antibodies significantly attenuated total HO (FIG. 1A) and attached HO (FIG. 1B) compared to isotype control by 9 weeks post-surgery (p<0.05). Acvr1 blocking antibodies significantly attenuated unattached HO (FIG. 1C) compared to isotype control by 13 weeks post-surgery (p<0.01).

In wild type mice induced with burn/tenotomy injury, ALK3-Fc reduced, but did not completely inhibit, HO by 60% (3.92 mm³ vs 1.63 mm³ total HO at week 13) consistent with previously published data (Agarwal et al. Mol Ther. Aug 2 2017;25(8): 1974-87) (Figure1A). Alk3-Fc significantly attenuated total HO (FIG. 1A) and attached HO (FIG. 1B) compared to isotype control by 6 weeks post-surgery (p<0.05). Alk3-Fc also significantly attenuated unattached HO (FIG. 1C) compared to isotype control by 13 weeks post-surgery (p<0.0001).

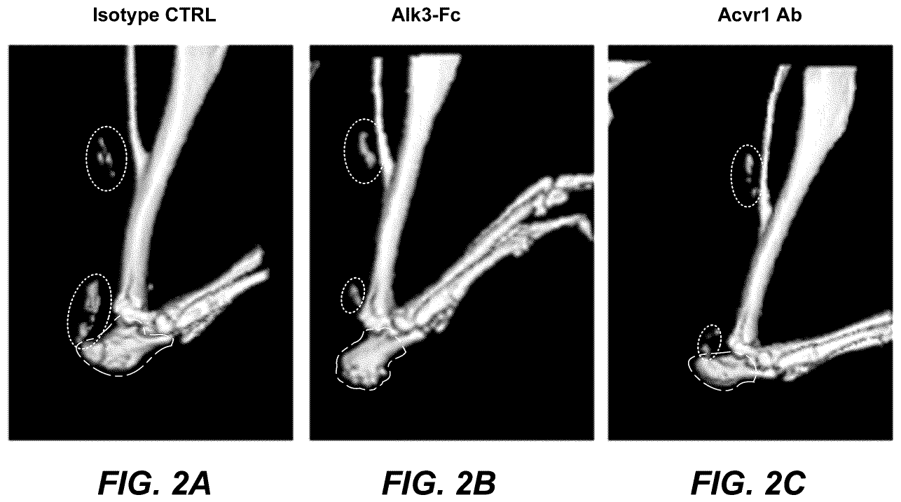

Images of HO volume in the injured hindlimb in WT mice as measured by total HO volume, attached HO (encircled by broken white lines) or unattached HO (encircled by short dashed white lines) are shown in FIGS. 2A-C. HO volumes by micro CT were significantly reduced after 13 weeks in both ALK3-Fc (FIG. 2B) and Acvr1 antibody (FIG. 2C) treated mice compared to isotype control (FIG. 2A). Total HO volume and attached HO volume was significantly reduced by 6 or 9 weeks post injury in the Alk3-Fc and Acvr1 antibody treated groups respectively.

In addition, WT mice were administered either activin A (n=15) antibodies or isotype control (n=15) starting concurrently with induction of injury in the burn/tenotomy injury model. HO volume was measured by microCT analysis 9 weeks post injury. HO volume in the injured hindlimb as measured by total volume, attached HO volume, or unattached HO volume was not significantly different between treatment groups. Activin A inhibition did not reduce HO formation or growth. Further stratification of floating and bone associated HO also did not demonstrate a difference between activin A treated and vehicle control treated animals (data not shown).

This example shows Acvr1 blocking antibodies significantly attenuate HO in an in vivo post-traumatic HO model; however, no significant effect of anti-activin A antibody was observed.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. (canceled)

2. The method of claim 33, wherein the full-length ACVR1 protein or mutant thereof is a full-length human ACVR1 protein or mutant thereof.

3. The method of claim 2, wherein the full-length human ACVR1 protein comprises amino acids 21-509 of SEQ ID NO: 341.

4. The method of claim 2, wherein the mutant ACVR1 protein comprises a mutation selected from the group consisting of ACVR1 L196P, delP197_F198insL, R202I, R206H, Q207E, R258S, R258G, G325A, G328E, G328R, G328W, G356D, and R375P of SEQ ID NO: 341.

5. The method of claim 4, wherein the isolated antibody or antigen-binding fragment thereof binds to ACVR1(R206H) protein and inhibits ACVR1(R206H)-mediated bone morphogenetic protein (BMP) signal transduction.

6. The method of claim 33, wherein the antibody or antigen-binding fragment thereof: (i) binds to cells expressing human ACVR1; and/or (ii) binds to ACVR1 and inhibits ACVR1-mediated bone morphogenetic protein (BMP) signal transduction.

7. The method of claim 6, wherein the antibody is a fully human monoclonal antibody.

8. The method of claim 33, wherein the antibody has one or more properties selected from the group consisting of: (a) is a fully human monoclonal antibody; (b) binds to human ACVR1 extracellular domain fused to an Fc (e.g., SEQ ID NO: 339) at 25° C. with a dissociation constant (K_{D})) of less than 60 nM, less than 12 nM, less than less than 2 nM, less than 1 nM, or less than 0.5 nM as measured in a surface plasmon resonance assay; (c) binds to human ACVR1 extracellular domain fused to mFc (SEQ ID NO: 339) at 37° C.with a dissociation constant (K_{D})) of less than 150 nM, less than 15 nM, less than less than 5 nM, less than 1.5 nM, or less than 1 nM as measured in a surface plasmon resonance assay; (d) binds to human ACVRI extracellular domain fused to myc-myc-hexahistag (e.g., SEQ ID NO: 338) at 25° C. with a K_{D} of less than 300 nM, less than 150 nM, less than 25 nM, less than 10 nM, less than 5 nM, less than 3 nM or less than 2 nM as measured in a surface plasmon resonance assay; (e) binds to human ACVR1 extracellular domain fused to myc-myc-hexahistag (e.g., SEQ ID NO: 338) at 37° C. with a K_{D} of less than 500 nM, less than 50 nM, less than 25 nM, less than 10 nM, as measured in a surface plasmon resonance assay; (f) does not bind mouse ACVRI or binds to mouse ACVR1 extracellular domain fused to myc-myc-hexahistag (e.g., SEQ ID NO: 340) at 25° C. with a K_{D} of greater than 500 nM, as measured in a surface plasmon resonance assay; (g) does not bind mouse ACVR1 or binds to mouse ACVRI extracellular domain fused to myc-myc-hexahistag (e.g., SEQ ID NO: 340) at 37° C. with a K_{D} of greater than 500 nM, as measured in a surface plasmon resonance assay; (k) binds to cells expressing human ACVRI protein or human ACVR1 (R206H) protein; (1) inhibits activation of cells expressing human ACVR1(R206H) by human Activin A with a ICso of less than 25 nM, as measured in a cell-based bioassay; (m) inhibits activation of cells expressing human ACVRI(R206H) by human BMP7 with a IC₅₀ of less than 20 nM, less than 5 nM, less than 3 nM, or less than 1 nM, or less than as measured in a cell-based bioassay; (m) significantly decreases serum hepcidin when administered to mice expressing human ACVR1 in place of mouse allele; (n) significantly increases serum iron levels when administered to mice expressing human ACVR1 in place of mouse allele; and/or (o) inhibits wild-type ACVR1 signaling when administered to mice expressing human ACVR1 in place of mouse allele; and (p) comprises a HCVR comprising an amino acid sequence selected from the group consisting of HCVR sequence listed in Table 1 and a LCVR comprising an amino acid sequence selected from the group consisting of LCVR sequences listed in Table 1.

9. The method of claim 33, wherein the antibody or antigen-binding fragment comprises three heavy chain complementarity determining regions (CDRs) (HCDR1, HCDR2 and HCDR3) contained within a heavy chain variable region (HCVR); and three light chain CDRs (LCDR1, LCDR2 and LCDR3) contained within a light chain variable region (LCVR), wherein the HCVR comprises an amino acid sequence selected from the group consisting of HCVR sequences listed in Table 1.

10. The method of claim 9, wherein the antibody or antigen-binding fragment thereof comprises a LCVR comprising an amino acid sequence selected from the group consisting of LCVR sequences listed in Table 1.

11. The method of claim 9, wherein the antibody or antigen-binding fragment thereof comprises:
(a) a HCDR1 domain having an amino acid sequence selected from the group consisting of SEQ ID NOs: 4, 24, 44, 78, 98, 130, 150, 168, 188, 205, 225, 257, and 321;
(b) a HCDR2 domain having an amino acid sequence selected from the group consisting of SEQ ID NOs: 6, 26, 46, 80, 100, 132, 152, 170, 207, 227, 243, 259, 275, 291, 303, and 323;
(c) a HCDR3 domain having an amino acid sequence selected from the group consisting of SEQ ID NOs: 8, 28, 60, 82, 102, 134, 154, 172, 191, 209, 229, 261, 305, and 325;
(d) a LCDR1 domain having an amino acid sequence selected from the group consisting of SEQ ID NOs: 12, 32, 50, 86, 106, 138, 158, 176, 195, 213, 233, 265, 279, 309, and 329;
(e) a LCDR2 domain having an amino acid sequence selected from the group consisting of SEQ ID NOs: 14, 34, 52, 64, 88, 108, 122, 140, 178, 215, 247, 281, 311, and 331; and
(f) a LCDR3 domain having an amino acid sequence selected from the group consisting of SEQ ID NOs: 16, 36, 66, 90, 110, 142, 160, 180, 197, 217, 235, 249, 267, 283, 313, and 333.

12. The method of claim 11, wherein the antibody or antigen-binding fragment thereof comprises a HCVR/LCVR amino acid sequence pair selected from the group consisting of SEQ ID NOs: 2/10, 22/30, 22/72, 42/48, 58/62, 76/84, 96/104, 116/119, 128/136, 148/156, 166/174, 186/193, 203/211, 223/231, 241/245, 255/263, 273/277, 289/293, 300/307, and 319/327.

13. The method of claim 12, wherein the antibody or antigen-binding fragment thereof comprises six CDRs selected from the group consisting of: (a) SEQ ID NOs: 44, 46, 28, 50, 52, and 36; and (b) SEQ ID NOs: 24, 46, 60, 50, 64, and 66.

14. The method of claim 13, wherein the antibody or antigen-binding fragment thereof comprises a HCVR/LCVR amino acid sequence pair selected from the group consisting of SEQ ID NOs: 42/48, and 58/62.

15. The method of claim 33, wherein the antibody or antigen-binding fragment comprises three heavy chain CDRs (HCDR1, HCDR2 and HCDR3) contained within a HCVR and three light chain CDRs (LCDR1, LCDR2 and LCDR3) contained within a LCVR; wherein the HCVR comprises:
(i) an amino acid sequence selected from the group consisting of SEQ ID NOs: 2, 22, 42, 58, 76, 96, 116, 128, 148, 166, 186, 203, 223, 241, 255, 273, 289, 300, and 319;
(ii) an amino acid sequence having at least 90% identity to the amino acid sequence selected from the group consisting of SEQ ID NOs: 2, 22, 42, 58, 76, 96, 116, 128, 148, 166, 186, 203, 223, 241, 255, 273, 289, 300, and 319;
(iii) an amino acid sequence having at least 95% identity to the amino acid sequence selected from the group consisting of SEQ ID NOs: 2, 22, 42, 58, 76, 96, 116, 128, 148, 166, 186, 203, 223, 241, 255, 273, 289, 300, and 319; or
(iv) an amino acid sequence selected from the group consisting of SEQ ID NOs: 2, 22, 42, 58, 76, 96, 116, 128, 148, 166, 186, 203, 223, 241, 255, 273, 289, 300, and 319, said amino acid sequence having no more than 12 amino acid substitutions, and the LCVR comprises:
(a) an amino acid sequence selected from the group consisting of SEQ ID NOs: 10, 30, 48, 62, 72, 84, 104, 119, 136, 156, 174, 193, 211, 231, 245, 263, 277, 293, 307, and 327;
(b) an amino acid sequence having at least 90% identity to the amino acid sequence selected from the group consisting of SEQ ID NOs: 10, 30, 48, 62, 72, 84, 104, 119, 136, 156, 174, 193, 211, 231, 245, 263, 277, 293, 307, and 327;
(c) an amino acid sequence having at least 95% identity to the amino acid sequence selected from the group consisting of SEQ ID NOs: 10, 30, 48, 62, 72, 84, 104, 119, 136, 156, 174, 193, 211, 231, 245, 263, 277, 293, 307, and 327; or
(d) an amino acid sequence selected from the group consisting of SEQ ID NOs: 10, 30, 48, 62, 72, 84, 104, 119, 136, 156, 174, 193, 211, 231, 245, 263, 277, 293, 307, and 327, said amino acid sequence having no more than 10 amino acid substitutions.

16. The method of claim 15, wherein the antibody or antigen-binding fragment thereof comprises a HCVR having an amino acid sequence selected from the group consisting of SEQ ID NOs: 2, 22, 42, 58, 76, 96, 116, 128, 148, 166, 186, 203, 223, 241, 255, 273, 289, 300, and 319.

17. The method of claim 15, wherein the antibody or antigen-binding fragment thereof comprises a LCVR having an amino acid sequence selected from the group consisting of SEQ ID NOs: 10, 30, 48, 62, 72, 84, 104, 119, 136, 156, 174, 193, 211, 231, 245, 263, 277, 293, 307, and 327.

18. The method of claim 15, wherein the antibody or antigen-binding fragment thereof of comprises three CDRs contained within a HCVR selected from the group consisting of SEQ ID NOs: 2, 22, 42, 58, 76, 96, 116, 128, 148, 166, 186, 203, 223, 241, 255, 273, 289, 300, and 319; and three CDRs contained within a LCVR selected from the group consisting of SEQ ID NOs: 10, 30, 48, 62, 72, 84, 104, 119, 136, 156, 174, 193, 211, 231, 245, 263, 277, 293, 307, and 327.

19. The method of claim 18, wherein the antibody or antigen-binding fragment comprises a HCVR/LCVR amino acid sequence pair selected from the group consisting of SEQ ID NOs: 2/10, 22/30, 22/72, 42/48, 58/62, 76/84, 96/104, 116/119, 128/136, 148/156, 166/174, 186/193, 203/211, 223/231, 241/245, 255/263, 273/277, 289/293, 300/307, and 319/327.

20. The method of claim 15, wherein the antibody or antigen-binding fragment thereof comprises:
(a) a HCDR1 domain having an amino acid sequence selected from the group consisting of SEQ ID NOs: 4, 24, 44, 78, 98, 130, 150, 168, 188, 205, 225, 257, and 321;
(b) a HCDR2 domain having an amino acid sequence selected from the group consisting of SEQ ID NOs: 6, 26, 46, 80, 100, 132, 152, 170, 207, 227, 243, 259, 275, 291, 303, and 323;
(c) a HCDR3 domain having an amino acid sequence selected from the group consisting of SEQ ID NOs: 8, 28, 60, 82, 102, 134, 154, 172, 191, 209, 229, 261, 305, and 325;
(d) a LCDR1 domain having an amino acid sequence selected from the group consisting of SEQ ID NOs: 12, 32, 50, 86, 106, 138, 158, 176, 195, 213, 233, 265, 279, 309, and 329;
(e) a LCDR2 domain having an amino acid sequence selected from the group consisting of SEQ ID NOs: 14, 34, 52, 64, 88, 108, 122, 140, 178, 215, 247, 281, 311, and 331; and
(f) a LCDR3 domain having an amino acid sequence selected from the group consisting of SEQ ID NOs: 16, 36, 66, 90, 110, 142, 160, 180, 197, 217, 235, 249, 267, 283, 313, and 333.

21. The method of claim 20, wherein the antibody or antigen-binding fragment thereof comprises six CDR amino acid sequences selected from the group consisting of: (a) SEQ ID NOs: 44, 46, 28, 50, 52, and 36; and (b) SEQ ID NOs: 24, 46, 60, 50, 64, and 66.

22. The method of claim 21, wherein the antibody or antigen-binding fragment thereof comprises a HCVR/LCVR amino acid sequence pair selected from the group consisting of SEQ ID NOs: 42/48, and 58/62.

23. The method of claim 33, wherein the antibody or antigen-binding fragment thereof is an isolated monoclonal antibody or antigen-binding fragment thereof that inhibits ACVR1-mediated and/or ACVR1(R206H)-mediated bone morphogenetic protein (BMP) signal transduction comprising three CDRs of a HCVR, wherein the HCVR has an amino acid sequence selected from the group consisting of SEQ ID NOs: 2, 22, 42, 58, 76, 96, 116, 128, 148, 166, 186, 203, 223, 241, 255, 273, 289, 300, and 319; and three CDRs of a LCVR, wherein the LCVR has an amino acid sequence selected from the group consisting of SEQ ID NOs: 10, 30, 48, 62, 72, 84, 104, 119, 136, 156, 174, 193, 211, 231, 245, 263, 277, 293, 307, and 327.

24. (canceled)

25. (canceled)

26. The method of claim 33, wherein the pharmaceutical composition comprises an isolated antibody or antigen-binding fragment thereof that binds to ACVR1 and a pharmaceutically acceptable carrier or diluent.

27. An isolated polynucleotide molecule comprising a polynucleotide sequence that encodes a HCVR of an antibody, wherein the antibody binds specifically to activin A receptor type 1 (ACVR1) protein and/or a mutant thereof, wherein the antibody interacts with one or more amino acids contained within the extracellular domain of ACVRI (amino acids 21-123 of SEQ ID NO: 341), and wherein the antibody binds to cells expressing full-length ACVR1 protein and/or a mutant thereof.

28. An isolated polynucleotide molecule comprising a polynucleotide sequence that encodes a LCVR of an antibody, wherein the antibody binds specifically to activin A receptor type 1 (ACVR1) protein and/or a mutant thereof, wherein the antibody interacts with one or more amino acids contained within the extracellular domain of ACVRI (amino acids 21-123 of SEQ ID NO: 341), and wherein the antibody binds to cells expressing full-length ACVR1 protein and/or a mutant thereof.

29. A vector comprising the polynucleotide sequence of claim 27.

30. A host cell expressing the vector of claim 29.

31. A method of producing an anti-ACVR1 antibody or antigen-binding fragment thereof, comprising growing the host cell of claim 30 under conditions permitting production of the antibody or fragment, and recovering the antibody or fragment so produced.

32. The method of claim 31, further comprising formulating the antibody or antigen-binding fragment thereof as a pharmaceutical composition comprising an acceptable carrier.

33. A method of treating, preventing or ameliorating at least one symptom or indication of a ACVR1-associated disease or disorder, the method comprising administering a pharmaceutical composition comprising a therapeutically effective amount of an antibody or antigen-binding fragment thereof of to a subject in need thereof, wherein the antibody or antigen-binding fragment thereof binds specifically to activin A receptor type 1 (ACVR1) protein and/or a mutant thereof, wherein the antibody or antigen-binding fragment thereof interacts with one or more amino acids contained within the extracellular domain of ACVR1 (amino acids 21-123 of SEQ ID NO: 341), and wherein the antibody or antigen-binding fragment thereof binds to cells expressing full-length ACVR1 protein and/or a mutant thereof.

34. The method of claim 33, wherein the ACVR1-associated disease or disorder is selected from the group consisting of heterotopic ossification, ectopic ossification, bone dysplasia, anemia, and diffuse intrinsic pontine glioma, optionally wherein the heterotopic ossification is post-traumatic heterotopic ossification.

35. The method of claim 33, wherein the pharmaceutical composition is administered prophylactically or therapeutically to the subject in need thereof.

36. The method of claim 33, wherein the pharmaceutical composition is administered in combination with a second therapeutic agent.

37. The method of claim 36, wherein the second therapeutic agent is selected from the group consisting of an anti-Activin A inhibitor, anti-BMP7 antibody or antigen binding fragment thereof, anti-ACVR2 antibody or antigen-binding fragment thereof, anti-inflammatory drugs, steroids, bisphosphonates, muscle relaxants, and retinoic acid receptor (RAR) gamma agonists, a lifestyle modification, and a dietary supplement.

38. The method of claim 33, wherein the pharmaceutical composition is administered subcutaneously, intravenously, intradermally, intraperitoneally, intramuscularly, or intracerebroventricularly.
